SEQUENCE LISTING (29 sequences)

SEQ ID NO: 1 - DNA, 20 bp, synthetic construct
  source 1..20: mol_type = other DNA
attgctccgctaaccccgtc

SEQ ID NO: 2 - DNA, 20 bp, synthetic construct
  source 1..20: mol_type = other DNA
gggttagcggagcaatgcgc

SEQ ID NO: 3 - DNA, 20 bp, synthetic construct
  source 1..20: mol_type = other DNA
ttccccaaatgccggttctg

SEQ ID NO: 4 - DNA, 17 bp, synthetic construct
  source 1..17: mol_type = other DNA
ccggttcatgcccatgc

SEQ ID NO: 5 - DNA, 17 bp, synthetic construct
  source 1..17: mol_type = other DNA
cgctatctgagcgctca

SEQ ID NO: 6 - DNA, 20 bp, synthetic construct
  source 1..20: mol_type = other DNA
ccccggacgatattgaacaa

SEQ ID NO: 7 - DNA, 21 bp, synthetic construct
  source 1..21: mol_type = other DNA
aagttcttgaggcactgcgaa

SEQ ID NO: 8 - DNA, 20 bp, synthetic construct
  source 1..20: mol_type = other DNA
ccggtacgttctccaaatcc

SEQ ID NO: 9 - DNA, 21 bp, synthetic construct
  source 1..21: mol_type = other DNA
atcgtggactatctgggcttc

SEQ ID NO: 10 - DNA, 21 bp, synthetic construct
  source 1..21: mol_type = other DNA
ttctggtgcattacatccgtc

SEQ ID NO: 11 - DNA, 20 bp, synthetic construct
  source 1..20: mol_type = other DNA
tcaagaacatgcggcaacac

SEQ ID NO: 12 - DNA, 19 bp, synthetic construct
  source 1..19: mol_type = other DNA
gggtactgtcgcgatacgg

SEQ ID NO: 13 - DNA, 18 bp, synthetic construct
  source 1..18: mol_type = other DNA
cggctgcgacaatgaccg

SEQ ID NO: 14 - DNA, 20 bp, synthetic construct
  source 1..20: mol_type = other DNA
caatgaggaacggcacgtag

SEQ ID NO: 15 - DNA, 20 bp, synthetic construct
  source 1..20: mol_type = other DNA
agtagcaaccaacgggaagg

SEQ ID NO: 16 - DNA, 20 bp, synthetic construct
  source 1..20: mol_type = other DNA
aaggtgctgacctaggcttg

SEQ ID NO: 17 - DNA, 20 bp, synthetic construct
  source 1..20: mol_type = other DNA
tcactgatggtgcttgcagt

SEQ ID NO: 18 - DNA, 23 bp, synthetic construct
  source 1..23: mol_type = other DNA
gccaggagatgattgttacagga

SEQ ID NO: 19 - DNA, 19 bp, synthetic construct
  source 1..19: mol_type = other DNA
tcgctctcccttcccgatg

SEQ ID NO: 20 - DNA, 23 bp, synthetic construct
  source 1..23: mol_type = other DNA
acctccatccctgctttcctaag

SEQ ID NO: 21 - DNA, 20 bp, synthetic construct
  source 1..20: mol_type = other DNA
aatttcagcgtcagctacac

SEQ ID NO: 22 - DNA, 21 bp, synthetic construct
  source 1..21: mol_type = other DNA
actagcaagcacccactaatc

SEQ ID NO: 23 - DNA, 21 bp, synthetic construct
  source 1..21: mol_type = other DNA
ccttagctcatcaggctcttt

SEQ ID NO: 24 - DNA, 20 bp, synthetic construct
  source 1..20: mol_type = other DNA
ggccgtttggctagtttgtt

SEQ ID NO: 25 - DNA, 18 bp, synthetic construct
  source 1..18: mol_type = other DNA
gagattggctggtcgcgt

SEQ ID NO: 26 - DNA, 22 bp, synthetic construct
  source 1..22: mol_type = other DNA
tgcccaggcgttgcgtgagaag

SEQ ID NO: 27 - DNA, 22 bp, synthetic construct
  source 1..22: mol_type = other DNA
acccgccacccttcgacagttc

SEQ ID NO: 28 - DNA, 20 bp, synthetic construct
  source 1..20: mol_type = other DNA
aagtcggtctctatgccgct

SEQ ID NO: 29 - DNA, 20 bp, synthetic construct
  source 1..20: mol_type = other DNA
ttgctgctctacagttatcc

(19) United States
(12) Patent Application Publication
(10) Pub. No.: US 2026/0007652 A1
(43) Pub. Date: Jan. 8, 2026

(54) Selective Modulators of AhR-Regulated Transcription and Method for Using Such Modulators to Treat Cancer

(71) Applicant: OREGON STATE UNIVERSITY, Corvallis, OR (US)

(72) Inventors: Siva K. KOLLURI, Corvallis, OR (US); Duc Nguyen BACH, Corvallis, OR (US); Daniel Jonathon ELSON, Corvallis, OR (US)

(73) Assignee: OREGON STATE UNIVERSITY, Corvallis, OR (US)

(21) Appl. No.: 19/120,590

(22) PCT Filed: Oct. 13, 2023
(86) PCT No.: PCT/US2023/035156

§ 371 Date: Apr. 11, 2025

Related U.S. Application Data
(60) Provisional application No. 63/416,255, filed on Oct. 14, 2022.

(51) Int. Cl. A61K 31/4745; A61K 31/18; A61K 31/198; A61K 31/366; A61K 31/4045; A61K 31/4184; A61K 31/435; A61K 31/4355; A61K 31/5025; A61K 31/506; A61K 31/519; A61K 31/5377; A61K 31/69; A61K 31/7048; A61K 31/706; A61K 31/7076; A61K 38/12; A61P 35/00

(52) U.S. Cl. CPC A61K 31/4745; A61K 31/18; A61K 31/198; A61K 31/366; A61K 31/4045; A61K 31/4184; A61K 31/435; A61K 31/4355; A61K 31/5025; A61K 31/506; A61K 31/519; A61K 31/5377; A61K 31/69; A61K 31/7048; A61K 31/706; A61K 31/7076; A61K 38/12; A61P 35/00

(57) ABSTRACT

Biologically active AhR ligand compounds, and pharmaceutical combinations comprising such compounds, tor treating proliferative cell diseases, such as cancer. Therapeutic combinations also are disclosed that comprise at least one AhR ligand compound together with a synergizing compound that, in combination with the AhR ligand compound, provides a synergistic biological effect. A particularly effective combination according to the present invention comprises (i) BBQ, 10-C1-BBQ and/or 11-C1-BBQ; and (ii) evoxine, citropten, or a combination thereof. Accordingly, the present invention also concerns a method comprising administering a therapeutically effective amount of at least one AhR ligand compound to a subject having a proliferating cell disease. Hie compound may be administered to the subject alone, as a pharmaceutical composition, or as a combination, such as a combination comprising at least one AhR ligand compound and an AhR ligand compound synergizer.

### CROSS-REFERENCE TO RELATED APPLICATION

This application claims the benefit of Application No. 63/416,255, filed Oct. 14, 2022, expressly incorporated herein by reference in its entirety.

### ACKNOWLEDGMENT OF GOVERNMENT SUPPORT

This invention was made with government support under contract numbers ES019000, ES016651 and ES007060 awarded by the National Institutes of Health. The United States government has certain rights in the invention.

### FIELD

The present application concerns selective modulators of AhR-regulated transcription, referred to herein as AhR ligand compounds, such as BBQ, and 10- and 11-Cl-BBQ, and embodiments of a method for administering such compounds, or combinations comprising such compounds, to treat proliferative cell diseases, such as cancer, either alone or in combination with one or more compounds that synergistically increase the biological effects of such AhR ligand compounds.

### BACKGROUND

Aryl hydrocarbon receptor (AhR) is a ligand-activated transcription factor belonging to the bHLH (basis helix-loop-helix)-PER-ARNT-SIM (bHLH/PAS) subfamily of the bHLH family of transcription factors. The binding of a wide range of both endogenous and exogenous small molecules activates AbR signaling that influences cellular outcomes in different developmental, immunological, and cancer-related contexts. Once activated, AhR translocates from the cytosol into the nucleus and heterodimerizes with AhR Nuclear Translocator (ARNT). Binding of the AhR/ARNT complex to specific promoter sequences recruits other transcriptional regulators to modulate the expression of downstream target genes.

Accumulating evidence indicates that the AbR can have context-dependent anti-tumorigenic effects depending on the biological activity of the ligands.

Recent studies showed that AhR exhibits tumor-suppressive effects against multiple cancers. As the fate of cancer cells can be influenced via AhR signaling, the AhR has been suggested as a molecular target for cancer drug development. Significant efforts have been focused on identifying AhR ligands with potent anticancer effects referred to herein as Select Modulators of AhR-regulated Transcription (SMAhRTs).

Triple-negative breast cancer (TNBC) represents a group of breast cancers defined by the absence of estrogen receptor (ER) and progesterone receptor (PR) expression, and lack of human epidermal growth factor receptor 2 (HER2) amplification. TNBCs are frequently aggressive, highly metastatic, and are recalcitrant to the targeted therapies employed in hormone positive and HER2-amplified breast cancers. Despite being superficially classified by the absence of these important immunohistochemical markers, TNBC is highly heterogeneous, and different subtypes exhibit wide variabilities in their molecular features, and response to therapies. Targeted treatments for TNBC are lacking. A clinical need therefore exists to identify novel therapeutic vulnerabilities for this subtype of cancer.

### SUMMARY

In one aspect, the present disclosure provides a method for treating cancer in a subject, comprising administering to a subject in need thereof a therapeutically effective amount of an AhR ligand compound and a synergizing compound that, in combination with the AhR ligand compound, provides a synergistic anti-cancer effect to the subject.

In another aspect, the present disclosure provides a method for treating cancer in a subject, comprising administering to a therapeutically effective amount of an AhR ligand compound to a subject in need thereof.

In a further aspect, the present disclosure provides a method for treating cancer in a subject, comprising administering to a subject in need thereof a therapeutically effective amount of an AhR ligand compound and a chemotherapeutic agent that, in combination with the AhR ligand compound, provides a synergistic anti-cancer effect to the subject.

The above methods utilize the AhR ligand compounds, synergizing compounds, chemotherapeutic agents, and their combinations as described herein.

Certain disclosed embodiments concern pharmaceutical compositions formulated for treating proliferative cell diseases, such as cancer. Disclosed pharmaceutical compositions comprise a therapeutically effective amount of an AhR ligand compound, such as an AhR ligand compound:
having a Formula II

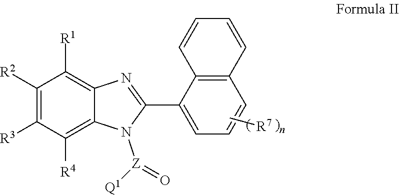

where n is 1, 2, 3, 4, 5, 6 or 7; R¹, R², R³, and R⁴ are independently H, halogen, CN, C1-C10 alkyl, C3-C10 cycloalkyl, C1-C6 alkoxy, SO₂R⁵, CO₂R⁵, or CONR⁵R⁶, or any one of R¹ and R², R² and R³, and R³ and R⁴ pairs, together with the carbon atoms to which they are attached, forms a five- or six-membered cycloalkenyl, heterocyclenyl, aryl or heteroaryl; R⁵ and R⁶ are independently H, C1-C10 alkyl, or C3-C10 cycloalkyl, or R⁵ and R⁶, together with the nitrogen atom to which they are attached, form a 5-membered ring or 6-membered ring; R⁷, at each occurrence, is independently H, halogen, CN, C1-C10 alkyl, C2-C6 alkenyl, C1-C10 heteroalkyl, C1-CIO heterocyclyl, C6-C10 aryl, C5-C10 heteroaryl, C1-C6 alkoxy, C1-C6 cycloalkyloxy, OCF₃, NR⁵R⁶, SCF₃, or C(O)NR⁵R⁶; and Q1 is C6-C10 aryl; C5-C10 heteroaryl; C5-C10 heterocyclyl; C1-C10 alkyl, or C3-C10 cycloalkyl;
or
a Formula III

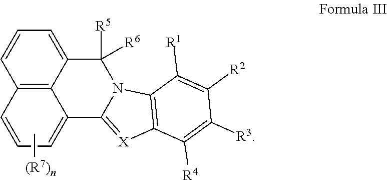

where n is 1, 2, 3, 4, 5, or 6; R¹, R², R³, and R⁴ are independently H, halogen, CN, C1-C10 alkyl, C3-C10 cycloalkyl, C1-C6 alkoxy, SO₂R⁸, CO₂R⁸, or CONR⁸R⁹, or any one of R¹ and R², R² and R³, and R³ and R⁴ pairs, together with the carbon atoms to which they are attached, forms a five- or six-membered cycloalkenyl, heterocyclenyl, aryl or heteroaryl ring; R⁵ and R⁶ are independently H, halogen, OH, or C1-C6 alkyl, or R⁵ and R⁶ taken together are ═O or ═S; R⁷, at each occurrence, is independently CN, C1-C6 alkyl, or halogen; R⁸ and R⁹ are H, C1-C10 alkyl, or C3-C10 cycloalkyl, or R⁸ and R⁹, together with the nitrogen atom to which they are attached, form a 5-membered ring or a substituted 6-membered ring; and X is N or CR¹⁰, where R¹⁰ is s H, halogen, C6-C10 aryl; C5-C10 heteroaryl, or C1-C6 alkyl. Particular disclosed embodiments concern pharmaceutical compositions wherein the AhR ligand compound is a benzo[de]benzoimidazoisoquinolinone, a naphthalenylbenzoimidazole, or a combination thereof, such as BBQ, 10-halo BBQ, and/or 11-halo-BBQ. Additional exemplary AhR ligand compounds according to the present invention are presented in FIG. 76.

Therapeutic combinations comprising such AhR ligand compounds together with a synergizing compound that, in combination with the AhR ligand compound, provides a synergistic biological effect, also are disclosed. The combination may be administered to a subject sequentially in any order, or substantially simultaneously, including as a composition. The AhR ligand compound may be any compounds according to formulas I-III, such as a benzo[de]benzoimidazoisoquinolinone, a naphthalenylbenzoimidazole, or a combination thereof, such as a benzo[de]benzoimidazoisoquinolinone compound having a formula IV

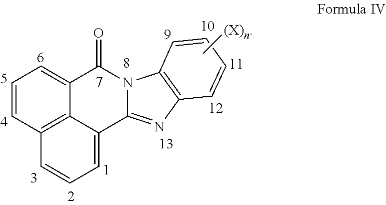

With reference to formula IV, n is 0, 1, 2, 3, or 4, and if X is present, X can be located on any carbon atom or atoms of the phenyl ring. X is preferably selected from hydrogen, fluorine, bromine, chlorine or iodine, was exemplified by BBQ, 10-halo BBQ, and/or 11-halo-BBQ.

Exemplary synergizer compounds may have a formula V

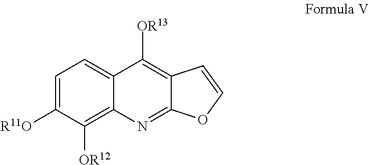

where R¹¹, R¹² and R¹³ are C1-C10 alkyl, including substituted C1-C10 alkyl where the substituents typically are selected from C1-C6 alkyl, hydroxyl, and combinations thereof. In certain embodiments, the R¹¹ substituent is a diol, such as

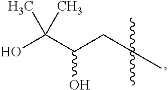 [SMILES: *CC(O)C(C)(C)O]

with a particular synergizer according to Formula V being Evoxine having the structure and stereochemistry indicated below

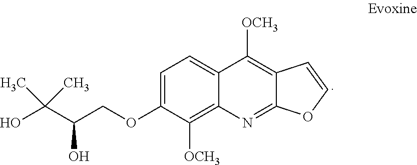 [SMILES: COc1c2ccoc2nc2c(OC)c(OC[C@@H](O)C(C)(C)O)ccc12]

Another class of useful synergizers according to the present invention include those having a formula VI

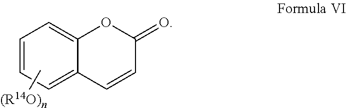

With reference to formula VI, R¹⁴ is C1-C6 alkyl, and n is 1, 2, 3 or 4, with one such compound, citropten, having R¹⁴=methyl, n=2.

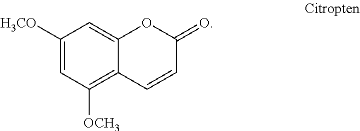 [SMILES: COc1cc(OC)c2ccc(=O)oc2c1]

Additional synergizers have been identified by screening known compounds, and such synergizers include ponatinib, melphalan, cladribine, mechloroethamine, vorinostat, ibrutinib, actinomycin D, decitabine, fludarabine, cabozantinib, dasatinib, belinostat, clofarabine, gemcitabine, pevonedistat, panobinostat, izaxomib, and teniposide.

A particularly effective combination according to the present invention comprises (i) BBQ, 10-Cl-BBQ and/or 11-Cl-BBQ; and (ii) evoxine, citropten, or a combination thereof. The combination may be formulated as a composition comprising at least one AhR ligand compound, such as a compound of FIG. 76, at least one synergizing compound that, in combination with the AhR ligand compound, provides a synergistic biological effect, and a pharmaceutically acceptable carrier, adjuvant and/or excipient.

AhR ligand compounds, and combinations comprising such compounds, have substantial biological effects, particularly anti-cancer effects, on proliferating cells but do not have the same effects on non-proliferating cells. Such biological effects include (1) decreasing proliferating cell viability, (2) apoptosis of proliferating or non-proliferating cells, (3) promotion of senescence of a cancer cell, (4) permanent or transient G1-phase cell cycle arrest of a cancer cell, (5) suppression of DNA replication, and/or (6) increased expression of cyclin-dependent kinase inhibitors. Accordingly, the present invention also concerns a method comprising administering a therapeutically effective amount of at least one AhR ligand compound to a subject having a proliferating cell disease. The compound may be administered to the subject alone, as a pharmaceutical composition, or as a combination, such as a combination comprising at least one AhR ligand compound and an AhR ligand compound synergizer. The combination administered may comprise, for example, an AhR ligand compound selected from those of FIG. 76, and particularly BBQ, 10-halo BBQ, and/or 11-halo-BBQ, and a synergizing compound selected from compounds according to formula V, compounds according to formula VI, Evoxine, Citropten, ponatinib, melphalan, cladribine, mechloroethamine, vorinostat, ibrutinib, actinomycin D, decitabine, fludarabine, cabozantinib, dasatinib, belinostat, clofarabine, gemcitabine, pevonedistat, panobinostat, izaxomib, teniposide, and any and all combinations thereof.

A particular method comprises inhibiting tumor progression by administering a therapeutically effective amount of BBQ, 10-halo BBQ, and/or 11-halo-BBQ to a subject.

A second particular disclosed method comprises administering a therapeutically effective amount of a combination to a subject, wherein the combination comprises:
an AhR ligand compound according to (1) Formula II

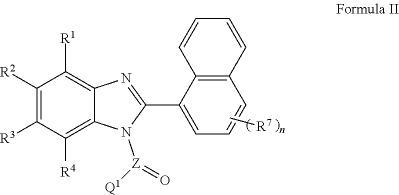

where n is 1, 2, 3, 4, 5 or 6; R¹, R², R³, and R⁴ are independently H, halogen, CN, optionally substituted C1-C10 alkyl, optionally substituted C3-C10 cycloalkyl, optionally substituted C1-C6 alkoxy, SO₂R⁵, CO₂R⁵, or CONR⁵R⁶, or any one of R¹ and R², R² and R³, and R³ and R⁴ pairs, together with the carbon atoms to which they are attached, forms an optionally substituted a five- or six-membered cycloalkenyl, heterocyclenyl, aryl or heteroaryl; R⁵ and R⁶ are independently H, optionally substituted C1-C10 alkyl, or optionally substituted C3-C10 cycloalkyl, or R⁵ and R⁶, together with the nitrogen atom to which they are attached, form an optionally substituted 5-membered ring or an optionally substituted 6-membered ring; R⁷, at each occurrence, is independently H, halogen, CN, optionally substituted C1-C10 alkyl, optionally substituted C2-C6 alkenyl, optionally substituted C1-C10 heteroalkyl, optionally substituted C1-CIO heterocyclyl, optionally substituted C6-C10 aryl, optionally substituted C5-C10 heteroaryl, optionally substituted C1-C6 alkoxy, optionally substituted C1-C6 cycloalkyloxy, OCF₃, NR⁵R⁶, SCF₃, or C(O)NR⁵R⁶; and Q1 is an optionally substituted C6-C10 aryl; optionally substituted C5-C10 heteroaryl; optionally substituted C5-C10 heterocyclyl; optionally substituted C1-C10 alkyl, or optionally substituted C3-C10 cycloalkyl;
or
(2) Formula III

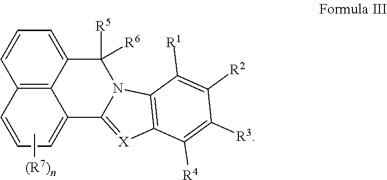

where n is 1, 2, 3, 4, 5, or 6; R¹, R², R³, and R⁴ are independently H, halogen, CN, C1-C10 alkyl, optionally substituted C3-C10 cycloalkyl, optionally substituted C1-C6 alkoxy, SO₂R⁸, CO₂R⁸, or CONR⁸R⁹, or any one of R¹ and R², R² and R³, and R³ and R⁴ pairs, together with the carbon atoms to which they are attached, forms an optionally substituted a five- or six-membered cycloalkenyl, heterocyclenyl, aryl or heteroaryl ring; R⁵ and R⁶ are independently H, halogen, OH, or C1-C6 alkyl, or R⁵ and R⁶ taken together are ═O or ═S; R⁷, at each occurrence, is independently CN, optionally substituted C1-C6 alkyl, or halogen; R⁸ and R⁹ are H, optionally substituted C1-C10 alkyl, or optionally substituted C3-C10 cycloalkyl, or R⁸ and R⁹, together with the nitrogen atom to which they are attached, form an optionally substituted 5-membered ring or an optionally substituted 6-membered ring; and n is 0, 1, 2, 3, 4, 5, or 6; and X is N or CR¹⁰, where R¹⁰ is s H, halogen, optionally substituted C6-C10 aryl; optionally substituted C5-C10 heteroaryl, or optionally substituted C1-C6 alkyl;
and
(3) a synergizer compound according to Formula V

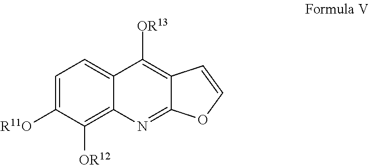

where R¹¹ R¹² and R¹³ are substituted C1-C10 alkyl, where the substituents are selected from C1-C6 alkyl, hydroxyl, and combinations thereof,
(4) a synergizer compound according to Formula VI

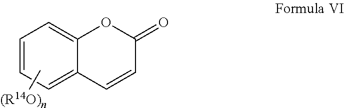

where R¹⁴ is C1-C6 alkyl, and n is 1, 2, 3 or 4, and/or
(5) a synergizer compound selected from ponatinib, melphalan, cladribine, mechloroethamine, vorinostat, ibrutinib, actinomycin D, decitabine, fludarabine, cabozantinib, dasatinib, belinostat, clofarabine, gemcitabine, pevonedistat, panobinostat, izaxomib, and teniposide.

A particular embodiment of a disclosed method concerns treating cancer by administering to a subject an effective amount of a combination comprising BBQ, 10-Cl-BBQ, and/or 11-Cl-BBQ, together with Evoxine, Citropten, or combination thereof.

Disclosed embodiments of a method for treating cancer in a subject may inhibit tumor progression or may kill a tumor cell. These biological effects are thought to arise from (1) promoting senescence of a cancer cell, (2) permanent or transient G1-phase cell cycle arrest of a cancer cell, (3) suppression of DNA replication, (4) increased expression of cyclin-dependent kinase inhibitors, and combinations thereof. For certain embodiments, these results may be AhR-dependent activities.

The foregoing and other objects, features, and advantages of the invention will become more apparent from the following detailed description, which proceeds with reference to the accompanying figures.

### BRIEF DESCRIPTION OF THE DRAWINGS

FIG. 1A is Western blot of whole cell lysates showing steady-state AhR protein expression in lung cancer cell lines.

FIG. 1B is a graph of lung cancer cell viability (%) versus 11-Cl-BBQ at indicated doses for 72 hours relative to vehicle control treated cells (set to 100%).

FIG. 1C is Western blot analysis of steady-state AhR protein expression in pooled cultures of H69AR cells transfected with three guide RNAs targeting human AhR (CR-AhR1, CR-AhR2, and CR-AhR3) or lentiCRISPRv2 control vector (CR-V2).

FIG. 1D is a graph of relative cell viability of AhR-proficient (CR-V2, gray), and two different pools of AhR-deficient (CR-AhR2, orange bars and CR-AhR3, red bars) H69AR lung cancer cells treated with 11-Cl-BBQ at indicated doses for 72 hours.

FIG. 1E is western blot analysis for the expression of AhR protein in clonal lines derived from H460 cells transfected with control vector (CR-V2) and/or sgRNA targeting AhR (CR-AHR3).

FIG. 1F is a graph of cell viability (%) of H460 AhR-proficient (CR-V@, gray) or H$\hat{ }) AhR-deficient (CR-AHR3, red) treated with 11-Cl-BBQ at indicated doses for 72 hours.

FIG. 1G provides images of colonies formed by H460 AhR-proficient (CR-V2) or H460 AhR-deficient (CR-AHR3) treated with 11-Cl-BBQ at indicated doses for 14 days.

FIG. 1H quantifies the number of colonies in FIG. 1G using OpenCFU software.

FIG. 2A provides a cell cycle analysis of H460 AHR wildtype (CR-V2) cells or AHR knockout (CR-AHR3) treated with 11-Cl-BBQ (2.5 μM) or vehicle control for 24 hours.

FIG. 2B quantifies cell cycle distribution of data presented by FIG. 2A for four biological replicates.

FIG. 2C is a graph of relative cell number (normalized to 0 h timepoint) versus time (hours) of H460 cells treated with 11-Cl-BBQ (2.5 μM) or vehicle control for 72 hours, followed by a second round of 11-Cl-BBQ (2.5 μM) or vehicle treatment at indicated time.

FIG. 2D is a histogram of senescent-associated β-galactosidase (sen-β-gal) in H460 AHR wildtype (CR-V2) cells or AHR knockout (CR-AHR3) treated with 11-Cl-BBQ (2.5 μM), Doxorubicin (Dox, 50 nM) or vehicle control for 5 days.

FIG. 2E quantifies sen-β-gal positive cell population of indicated cells and treatments where *; adjusted p value <0.05, **: adjusted p value <0.01, ***: adjusted p value <0.001, ns: not statistically significant.

FIG. 3A is schematic of a zebrafish xenograft experiment: H460 AHR sufficient cells (CR-V2) or AHR deficient cells (CR-AHR3) treated with 11-Cl-BBQ (1 or 5 μM), or vehicle control (0.1% DMSO) for 72 hours were dyed with CMdil and injected into the yolk sac of zebrafish embryo; the growth of the cells was monitored at 1-day post-injection (1 dp) and again at 4 dp using high content imager.

FIG. 3B provides relative tumor size of indicated treatment groups (about 20 fish/group) at 4 dp compared to 1 dp.

FIG. 3C provides representative images of H460 zebrafish xenograft composed of zebrafish (bright field) and H460 cell (red channel) and overlayed images.

FIG. 4A provides expression information (TPM, transcripts per kilobase million) of well-known targets of AHR (CYP1A1, CYP1B1, AHRR and TIPARP) in H460 AHR wildtype (CR-V2) or AHR knockout (CR-AHR3) treated with 11-Cl-BBQ (5 μM), or vehicle control (0.1% DMSO) for 4 or 12 hours measured by RNA-seq method.

FIG. 4B is a Venn diagram showing the number of genes that changed ≥2 fold with 11-Cl-BBQ treatment compared to vehicle treatment in an AHR-dependent manner at indicated times.

FIG. 4C provides expression information of select genes in H460 AHR wildtype and AHR knockout treated with 11-Cl-BBQ (5 μM) or vehicle control for 4 hours, measured by RNA-seq and qPCR methods.

FIG. 4D provides information concerning all canonical pathways that enriched upon 11-Cl-BBQ treatment for 12 hours in AHR wildtype (FDR<25%, blue bars) and in AHR knockout cells (red bars) by gene set enrichment analysis (GSEA).

FIG. 4E provides information concerning top 9 gene ontology terms of a cluster related to cell cycle regulation (adj p-val<0.01) revealed by Gene Ontology analysis of differentially expressed transcripts upon 11-Cl-BBQ treatment for 12 hours.

FIG. 5A is a western blot analysis of p53 protein levels in H460 cells treated with 11-Cl-BBQ (5 μM) at the indicated time.

FIG. 5B is a western blot analysis of p53 protein levels in H460 cells treated with TCDD (30 nM) at the indicated time.

FIG. 5C is a western blot analysis of p53 protein levels in H460 p53 wildtype (WT) and p53 knockout (KO) treated with 11-Cl-BBQ (2.5 μM), TCDD (30 nM), or vehicle control for 9 hours.

FIG. 5D is a representative histogram illustrating cell cycle distribution of H460 p53 WT or p53 KO cells treated with 11-Cl-BBQ (2.5 μM) or vehicle control for 24 hours.

FIG. 5E provides quantification information concerning cell cycle distribution of H460 p53 WT or p53 KO cells treated with 11-Cl-BBQ (2.5 μM) or vehicle control for 24 hours.

FIG. 5F is a representative histogram and quantification of sen-β-gal in H460 p53 WT and p53 KO treated with 11-Cl-BBQ (2.5 UM) or vehicle control for 5 days, where the media on cells was changed on day 3 and were treated with 11-Cl-BBQ or vehicle.

FIG. 6A is graph of change versus treatment time (hours) for expression of p27 (CDKN1B) mRNA measured by qPCR in H460 cells treated with 11-Cl-BBQ (2.5 M) or TCDD (30 nM) relative to vehicle control treatment (0.1% DMSO) at indicated times.

FIG. 6B is a graph of immunoprecipitation (%) versus treatment illustrating enrichment of AHR at CYP1A1 promoters in H460 cells after treatment with 11-Cl-BBQ (2.5 μM), TCDD (30 nM), or vehicle control for one hour measured by qPCR after chromatin immunoprecipitated (IPed) with AHR specific antibody (AHR) or immunoglobulin G control (IgG).

FIG. 6C is a graph of immunoprecipitation (IPed, %) versus treatment illustrating enrichment of AHR at p27 promoters in H460 cells after treatment with 11-Cl-BBQ (2.5 μM), TCDD (30 nM), or vehicle control for one hour measured by qPCR after chromatin immunoprecipitated (IPed) with AHR specific antibody (AHR) or immunoglobulin G control (IgG).

FIG. 6D is a western blot analysis of p27 protein in H460 AHR proficient (CR-V2) and AHR deficient (CR-AHR3) cells treated with 11-Cl-BBQ at indicated doses for 72 hours.

FIG. 6E is a western blot analysis of p27 protein levels in H460 cells transfected with CRIPSR-cas9 with sgRNA specific for p27 gene (CR-p27) or CRISPR-cas9 control vector (CR-V2) treated with 11-Cl-BBQ (2.5 μM) for 24 hours.

FIG. 6F is a graph of percent of cells versus cell cycle distribution of control H460 cells expressing p27 (CR-V2) or not expressing p27 (CR-p27) treated with 11-Cl-BBQ (1 μM) or vehicle control for 24 hours.

FIG. 6G is a graph of SA-β-gal positive (%) quantifying sen-β-gal in H460 p27 expressing (CR-V2) and p27 non-expressing (CR-p27) cells treated with 11-Cl-BBQ.

FIG. 7A provides the structure of 11-Cl-BBQ.

FIG. 7B is a graph of AHR activation (fold) versus concentration for 11-Cl-BBQ at the indicated concentration and by TCDD (1 nM) as measured by XRE-luciferase reporter assay.

FIG. 7C provides Sanger sequencing data of H460 CR-AHR3 clonal line aligned to reference sequence for exon 5 of human AbR gene and AHR sgRNA3.

FIG. 7D shows premature stop codons (*) in AHR coding sequence from H460 CR-AHR3 clone.

FIG. 7E is graph of relative expression of AHR mRNA in H460 CR-AHR3 cells compared to H460 CR-V2 cells.

FIG. 8A provides Annexin V staining of H460 AHR wildtype (CR-V2) cells or AHR knockout (CR-AHR3) treated with 11-Cl-BBQ (1 and 10 μM) or vehicle control for 72 hours.

FIG. 8B provides representative SA-β-gal staining (FITC) versus side scatter (SSC) dot blot (with gating for SA-β-gal) of H69AR treated with vehicle or 11-Cl-BBQ (1μ) for 120 hours.

FIG. 8C quantifies SA-β-gal positive cell populations from biological replicates for each treatment, with the p-value from a two-tailed ttest shown.

FIG. 9A is a graph of fold change (relative to CR-V2 vehicle) showing expression of p21 (CDKN1A) mRNA in H460 AHR wildtype (CR-V2) or AHR knockout (CR-AHR3) cells treated with 11-Cl-BBQ (5 μM) or vehicle control for 4 and 12 hours measured by RNA-seq.

FIG. 9B is a western blot showing the expression of p21 protein in H460 AHR wildtype or AHR knockout cells treated with 11-Cl-BBQ (2.5 μM), TCDD (30 nM) or vehicle control.

FIG. 9C is a graph of fold change (relative to CR-V2) showing expression of p21 mRNA in H460 p53 wildtype (WT) or p53 knockout (KO) cells treated with 11-Cl-BBQ (2.5 μM) or vehicle control for 4 hours measured by qPCR.

FIG. 10 is a western blot of AbR expression in vector control (AhR WT) and cr-AhR (AhR KO) MDA-MB-468 cells.

FIG. 11 is a plot of 11-Cl-BBQ analogs screened in MDA-MB-468 cells with or without AhR expression, with bars representing AhR-dependent inhibition of cell viability following 72-hour exposure at 10 μM.

FIG. 12 is a graph of cell number (%) versus treatment for AhR WT and AhR KO MDA-MB-468 cells.

FIG. 13 is a graph of AhR activation (fold) versus concentration of Analog 523 (BBQ) illustrating induction of xenobiotic-response element reporter in Hepa-1 cells after 16-hour treatment.

FIG. 14 illustrates relative cell cycle distribution of MDA-MB-468 cells (AhR WT and KO) treated with 100 nM of Analog 523 for 24 hours.

FIG. 15A is a two-dimensional colony forming assay in AhR WT and KO MDA-MB-468 cells treated with 10 nM of Analog 523.

FIG. 15B quantifies colony number for the colony forming assay illustrated by FIG. 15A.

FIG. 16A provides images acquired at 20× of clones from a three-dimensional soft-agar assay in AhR WT and AhR KO MDA-MB-468 cells treated with 10 nM of Analog 523.

FIG. 16B quantifies the number of clones per field relative to vehicle control from a three-dimensional soft-agar assay in AhR WT and AhR KO MDA-MB-468 cells treated with 10 nM of Analog 523.

FIG. 17A is graph of cell viability (%) versus treatment quantifying MDA-MB-468 spheroid cell viability (AhR WT and AhR KO) following exposure to Analog 523 for 72 hours.

FIG. 17B shows images of MDA-MB-468 spheroids.

FIG. 18 is a graph of Annexin-V Positive (%) versus treatment quantifying apoptosis induced by Analog 523 in AhR WT and AhR KO cells following 48 hours exposure.

FIG. 19 is a graph of cell number (%) quantifying cell viability in primary human mammary epithelial cells (HMEC) and non-malignant mammary epithelial cells (MCF10A) following 72-hour exposure to Analog 523.

FIG. 20 is a graph of cell number (%) versus concentration of Analog 523 quantifying cell viability in non-tumorigenic HEK293T cells, and primary normal human fibroblasts following 72-hour exposure to Analog 523.

FIG. 21 is graph of cell number (%) versus concentration of Analog 523 quantifying cell viability from a panel of triple-negative breast cancer cells, estrogen-receptor positive, and HER2-overexpressing breast cancer cells exposed to Analog 523 for 72 hours.

FIG. 22 is a graph of Annexin-V positive (%) versus treatment measuring apoptosis (Annexin-V positivity) induced by 1 μM Analog 523 in HCC70 TNBC cells (Basal A subtype).

FIG. 23 is a graph of Annexin-V positive (%) versus treatment measuring apoptosis (Annexin-V positivity) induced by 1 μM Analog 523 in HCC1187 TNBC cells (Basal A subtype).

FIG. 24 is a western Blot of AhR expression in two isolated populations of TNBC stem cells (ST1/ST2).

FIG. 25 are images of immunocytochemical staining of AhR in ST2 cells treated with the AhR ligand TCDD to induce receptor nuclear translocation.

FIG. 26 provides RT-qPCR of AhR target genes in ST1 and ST2 cells showing that AhR transcriptional activity is functional.

FIG. 27 is a graph of cell number (%) versus treatment illustrating cell viability of ST1 and ST2 cells treated with Analog 523 for 48 hours.

FIG. 28 is a western Blot of AbR expression in ST2 cells transfected with scramble siRNA or siRNA against AhR transcript.

FIG. 29 is viability (% vehicle) versus treatment (100 nM, 1 μm) illustrating cell viability of ST2 cells treated with Analog 523 after siRNA-mediated knockdown of AhR.

FIG. 30 illustrates gene ontology (GO TERM) analysis and gene set enrichment analysis that was used to determine the AhR-dependent biological processes and pathways downstream of Analog 523, and further illustrates induction of well-established AhR transcriptional targets that were observed, including TIPARP, CYP1A1, CYP1A2, and CYP1B1.

FIG. 31 is a Venn-diagram of upregulated genes following Analog 523 treatment for 4 hours and 12 hours in MDA-MB-468 AhR WT but not AhR KO cells. Represented genes exhibited fold changes greater than 2 and had adjusted p-values <0.05.

FIG. 32 is a bubble plot showing enriched Hallmark Gene Sets from the Molecular Signatures Database (MolSigDB) after 4- and 12 hours treatment with Analog 523. DEGS analyzed exhibited fold changes greater than 1.5 and adjusted p-values <0.05.

FIG. 33 is a bubble plot displaying enriched pathways (WikiPathways) following 4- and 12 hours treatment with Analog 523. DEGS analyzed exhibited fold changes greater than 1.5 and adjusted p-values <0.05.

FIG. 34 provides fold change mRNA illustrating enriched Apoptosis Hallmark genes at 4 hours post-treatment with Analog 523. DEGS analyzed exhibited fold changes greater than 1.5 and adjusted p-values <0.05.

FIG. 35 provides fold change mRNA illustrating enriched p53 signaling Hallmark genes common to both 4- and 12-hours post-treatment with Analog 523 (DEGS analyzed exhibited fold changes greater than 1.5 and adjusted p-values <0.05).

FIG. 36 is a Venn-diagram of downregulated genes following Analog 523 treatment for 4 hours and 12 hours in MDA-MB-468 AhR WT but not AbR KO cells (represented genes exhibited fold changes greater than 2 and had adjusted p-values <0.05).

FIG. 37 is a bubble plot showing enriched Hallmark Gene Sets from the Molecular Signatures Database (MolSigDB) after 4- and 12 hours treatment with Analog 523, where DEGS analyzed exhibited fold changes greater than 1.5 and adjusted p-values <0.05.

FIG. 38 is a bubble plot displaying enriched pathways (WikiPathways) following 4- and 12 hours treatment with Analog 523, where DEGS analyzed exhibited fold changes greater than 1.5 and adjusted p-values <0.05.

FIG. 39 is a western blot of HepG2 clones that were transfected with vector control or different guide RNAs targeting AhR genomic loci to disrupt AhR gene expression.

FIG. 40 is a graph of cell number (%) versus treatment illustrating cell viability of HepG2 cells (vector control or AhR-2 transfected) treated with Analog 523 (0.4 μM, 2 μm, and 10 μM) for 72 hours.

FIG. 41 is a graph of Annexin-V positive (%) versus treatment quantifying apoptosis (Annexin-V staining positivity) in HepG2 cells following 48-hour treatment with Analog 523 (2 μm).

FIG. 42 is a Western blot of AhR expression in mouse Hepa-1 cells expressing AbR, or an AhR-deficient clone Hepa-1 AhR-deficient.

FIG. 43 is a graph of cell number versus treatment illustrating cell viability of Hepa-1 or Hepa-1 AhR-deficient cells treated with Analog 523 for 48 hours.

FIG. 44 is a graph of Annexin-V positive (%) versus treatment quantifying apoptosis (Annexin-V staining positivity) in Hepa-1 cells treated with Analog 523 for 48 hours.

FIG. 45 provides colony images (top panel) for a colony forming assay, and corresponding quantification (bottom panel), with Hepa-1 or Hepa-1 AhR-deficient cells treated with Analog 523 (50 nM).

FIG. 46 provides transcriptional profiling by RT-qPCR Apoptosis array of Hepa-1 cells treated with 300 nM Analog 523 for 24 hours.

FIG. 47 lists chemotherapy drugs screened at 3 different concentrations (0.1, 1 and 10 μM) where purple indicates sensitization of MDA-MB-468 cells to chemotherapeutics when co-treated with 50 nM of Analog 523, where values shown are % cell viability of the screened drug and Analog 523 combination subtracted from that of drug plus vehicle (n=3).

FIG. 48 lists the effects of certain exemplary therapeutics (cladribine, ponatinib, actinomycin D, melphalan, and teniposide) when used alone, in combination with vehicle, Analog 523 alone, and Analog 523 used in combination with these exemplary therapeutics.

FIG. 49 concerns dose studies in mice using 2 mg/Kg for intravenous administration that resulted in detecting up to 1.6 μM of 11-Cl-BBQ in mouse serum.

FIG. 50 concerns dose studies in mice using 8 mg/Kg for oral administration that resulted in detecting 3 μM of 11-Cl-BBQ in mouse serum.

FIG. 51 is a graph of cell viability versus treatment showing the effects of using 11-Cl-BBQ alone and in combination with Evoxine or in combination with Citropten, establishing that these combinations have synergistic effects.

FIG. 52 is a bar graph of cell viability versus treatment and concentration showing that the combination of Analog 523, an AhR ligand according to the present invention, when used in combination with Evoxine and Citropten provided a synergistic reduction in cell viability.

FIGS. 53A-53E are histograms of cell count versus Annexin-V positivity establishing that 11-Cl-BBQ in combination with Citropten and 11-Cl-BBQ in combination with Evoxine induced potent apoptotic responses.

FIG. 54A is a graph of cell number (%) versus log of the 523 Analog concentration for 523 alone or 523 plus 1 μM Citropten, 3 μM Citropten, or 10 μM Citropten.

FIG. 54B is a graph of cell number (%) versus log of Citropten alone, or Citropten plus 10 nM Analog 523, Citropten plus 50 nM Analog 523, or Citropten plus 100 nM Analog 523.

FIG. 55A is a graph of cell number (%) versus log of the Analog 523 concentration for 523 alone, or Analog 523 plus 1 μM Evoxine, Analog 523 plus 3 μM Evoxine, or Analog 523plus 10 μM Evoxine.

FIG. 55B is a graph of cell number (%) versus log of Evoxine alone, or Evoxine plus 10 nM Analog 523, Evoxine plus 50 nM Analog 523, or Evoxine plus 100 nM Analog 523.

FIG. 56 provides Annexin-V apoptosis staining data for 0.01 μM Analog 523, Evoxine alone, and 0.01 μM Analog 523+ μM Evoxine showing a 24% increase in apoptotic response for the combination 0.01 μM Analog 523+Evoxine for Hepatoma (mouse) cells.

FIG. 57 provides Annexin-V apoptosis staining data for 0.3 μM Analog 523 alone, 10 μM Evoxine alone, and 0.3 μM Analog 523+10 μM Evoxine showing an increase in apoptotic response for the combination 0.3 μM analog 523+10 μM Evoxine for human Hepatocellular carcinoma cells.

FIG. 58 provides cell viability data for 10 μM Citropten, 0.1 μM analog 435, and the combination of 10 μM Citropten+0.1 μM analog 435 for SKBR-3 HER2+ breast cancer cells showing a 22.6% decrease in cell viability.

FIG. 59 provides cell viability data for 10 μM Evoxine, 0.1 μM analog 435, and the combination of 10 μM Evoxine+0.1 μM analog 435 for SKBR-3 HER2+ breast cancer cells showing a 20.3% decrease in cell viability.

FIG. 60 is a bar graph showing colony formation (%) versus treatment, namely 10 μM 11-Cl-BBQ. 2.5 μM Evoxine and 10 μM 11-Cl-BBQ+2.5 μM Evoxine.

FIG. 61 provides bar graphs of cell number (%) (for MDA-MD-468 and MCF10A cell lines) versus treatment (10 nM Analog 523, 10 μM Evoxine, and 10 nM analog 523+10 μM Evoxine).

FIG. 62 provides bar graphs of cell viability (%) for human mammary epithelial cells versus treatment, namely 100 nM Analog 523, 100 nM 11-Cl-BBQ, 10 nM TCDD, 10 μM Evoxine, Analog 523 combination, 11-Cl-BBQ combination, and TCDD combination establishing that the tested Evoxine combination does not inhibit non-cancerous breast epithelial cell growth.

FIG. 63 compares MDA-MB-468 cell viability for 11-Cl-BBQ (11BBQ) alone, specified therapeutic drug alone, and the combination of 11-Cl-BBQ and the drug. MDA-MB-468 cells were seeded in 384-well plates and treated with either 250 nM 11-Cl-BBQ, 10 UM of each specified compound, or 250 nM 11-Cl-BBQ combined with 10 μM of the compound. Cells were incubated for 72 hours before harvesting for cell viability assay.

FIGS. 64A-64H show anti-cancer effects of Analog 523 in hepatocellular carcinoma. A) Western Blot of HepG2 clones that were transfected with vector control or different guide RNAs targeting AhR genomic loci to disrupt AhR gene expression. B) Cell viability of HepG2 cells (vector control or AhR-2 transfected clone 3-AhR KO) treated with Analog 523 at the indicated concentrations for 72 hours. C) Quantification of AhR-dependent apoptosis (Annexin-V staining positivity) in HepG2 cells following 48-hour treatment with Analog 523. D) Western blot of AhR expression in mouse Hepa-1 cells expressing AhR (AhR WT), or an AhR-deficient clone Hepa-1 AhR-deficient. E) Cell viability of Hepa-1 AhR WT or Hepa-1 AhR-deficient cells treated with Analog 523 for 48 hours. F) Quantification of apoptosis (Annexin-V staining positivity) in Hepa-1 cells treated with Analog 523 for 48 hours. G) Colony forming assay (top panel), and corresponding quantification (bottom panel), with Hepa-1 or Hepa-1 AhR-deficient cells treated with Analog 523. H) Transcriptional profiling by RT-qPCR Apoptosis array of Hepa-1 cells treated with 300 nM Analog 523 for 24 hours.

FIGS. 65A-65C show anti-cancer effects of Analog 523 in non-small cell lung cancer. A) Western Blot of AhR expression in NCI-H460 lung cancer cell clones. CRISPR-Cas9 gene editing was used to delete AhR expression (AhR KO). B) Annexin-V staining of NCI-H460 AhR-expressing (AhR WT) or AhR KO cells treated with 5 μM of Analog 523 for 48 hours. C) Cell viability of NCI-H460 AhR-expressing or AhR KO lung cancer cells treated with Analog 523 for 48 hours.

FIGS. 66A-66E show the anti-cancer effects of representative 11-Cl-BBQ compounds on H460 cells.

FIGS. 67A-67E show a phenotypic screen for representative 11-Cl-BBQ analogs (848 and 849) with AHR-dependent anticancer effects. (A) Western blot showed the expression of AHR protein in H460 CR-V2 (AHR wildtype) and H460 CR-AHR3 (AHR knockout) cells; GADPH was used as a loading control. (B) Schematic of cell viability-based phenotypic screening for 11-Cl-BBQ analogs with AHR-dependent anticancer effects at 100 nM, 1 μM, and 10 μM; AHR-dependency defined by the cell viability of H460 AHR-knockout cells subtracted for the viability of H460 AHR-wildtype cells. (C) Summary of the screening results at 100 nM dose; analogs with AHR dependency >Mean+2 standard deviation (SD) of AHR-dependency of representative analogs considered as bits (red dots); additional representative hits were labeled here and in FIGS. 66B-66E. (D) Relative cell viability (vehicle-treated as 100%) of H460 AHR-wildtype (WT) and AHR-knockout (KO) treated with two analogs, PRXS-848 and PRXS-849, identified in (C) along with the parental lead 11-Cl-BBQ at the indicated doses for 72 hours. (E) Representative images of cell colonies formed by H460 AHR-wildtype (WT) and AHR-knockout (KO) treated with PRXS-848 (100 nM), PRXS-849 (100 nM), or vehicle control (0.1% DMSO) for two weeks.

FIGS. 68A-68H show induced AHR-dependent cell death for representative 11-Cl-BBQ analogs (848 and 849). (A) Expression of AHR protein in clonal lines H460 AHR wildtype (WT, CR-V2), H460 AHR-knockout-1 (KO-1, CR-AHR1), and H460 AHR-knockout-2 (KO-2, CR-AHR3) examined by Western blot; GADPH was used as a loading control. (B and C) Cell viability of AHR wildtype and AHR knockout lines treated with PRXS-848 (B) and PRXS-849 (C) at the indicated concentrations for 48 hours in 2D culture in PRMI 10% FBS media. (D) Representative images tumor spheroids formed by H460 AHR-wildtype (WT) and AHR-knockout-2 (KO-2) treated with PRXS-848 (250 nM), PRXS-849 (200 nM), or vehicle control (0.1% DMSO) for 72 hours. (E and F) Viability of AHR wildtype (WT) and AHR knockout (KO-2) tumor spheroids (as in D) treated with PRXS-848 (E) and PRXS-849 (F) at the indicated concentrations for 72 hours. (G and H) Representative images of H460 AHR-wildtype (WT) and AHR-knockout-2 (KO-2) cells treated with PRXS-848 (250 nM), PRXS-849 (200 nM), or vehicle control (0.1% DMSO) for 48 hours in RPMI 10% FBS (G), and cell death (annexin V positive) was quantified by annexin V staining assay (H).

FIGS. 69A-69D show representative 11-Cl-BBQ analogs (848 and 849) as non-classical AHR ligands. (A) Stability of AHR protein in the H460 whole cell lysate in the presence or absence of 11-Cl-BBQ (5 μM), PRXS-848 (10 μM), and PRXS-849 (10 μM) at the indicated temperatures; GADPH was used as a loading control. (B) Localization of AHR protein in the H460 treated with 11-Cl-BBQ (100 nM), PRXS-848 (250 nM), PRXS-849 (250 nM), or vehicle control (0.1% DMSO) for two hours; top horizontal panel: AHR protein staining using an AHR-specific antibody conjugated with FITC (green) fluorophore; middle panel: DAPI staining; bottle panel: overlaid images from the top panel and the middle panel in the same column. (C) AHR activation (measured by XRE-luciferase reporter assay in Hepa1 cells) of the parental lead 11-Cl-BBQ and analogs PRXS-848 and PRXS-849 at the indicated concentrations for 16 hours. (D) AHR activation of 11-Cl-BBQ cotreated with a known AHR antagonist CH223191 (1 μM) or 11-Cl-BBQ analogs PRXS-848 and PRXS-849 at the indicated concentrations for 16 hours.

FIGS. 70A-70D show cancer selective killing effects for representative 11-Cl-BBQ analogs (848 and 849). (A and B) Expression of AHR in lung adenocarcinoma (LUAD), lung squamous cell carcinoma (LUSC) primary tumors, and normal lung tissues from The Cancer Genome Altas (accessed through the http://ualcan.path.uab.edu/portal). (C and D) Cell viability of H460 lung cancer cells and NHBE primary human bronchial epithelial cells treated with PRXS-848, PRXS-849, or vehicle control (0.1% DMSO) for 72 hours.

FIGS. 71A-71C show representative AhR ligand 11-Cl-BBQ induces AhR-dependent anti-proliferative effects in TNBC cells. A) Viability of control AhR-expressing (AhR WT) versus AhR KO MDA-MB-468 cells treated with 11-Cl-BBQ for 72 hours. B) Viability of multiple additional TNBC cell lines treated with 11-Cl-BBQ for 72 hours. C) Colony forming assay demonstrating AR-dependent inhibition of colony growth by 11-Cl-BBQ in MDA-MB-468 cells.

FIGS. 72A-72C presents the characterization of combinations on TNBC cell viability and cell death. Quantitation of Annexin-V staining of MDA-MB-468 cells treated with either 10 μM evoxine, 10 μM citropten, 0.25 μM 11-Cl-BBQ, or corresponding combinations for 72 hours (A and B, respectively). Quantifications represent averages of three independent experiments. C) Colony forming assay of MDA-MB-468 AhR-expressing and AhR KO cells treated with 100 nM Evoxine, 500 nM Citropten, 100 μM 11-Cl-BBQ, or combinations of each compound with 11-Cl-BBQ.

FIGS. 73A and 73B present the characterization of Evoxine- and 11-Cl-BBQ-mediated combination effects. A) Dose response curve of MDA-MB-468 cell viability following 48-hour treatment with Evoxine alone (blue line) versus Evoxine combined with 11-Cl-BBQ fixed at a concentration of 10 nM. B) Dose-response curve of MDA-MB-468 cell viability following 48-hour treatment with 11-Cl-BBQ alone at different concentrations (blue line) or 11-Cl-BBQ combined with 3 μM Evoxine.

FIGS. 74A and 74B present the characterization of Citropten- and 11-Cl-BBQ-mediated combination effects. A) Dose response curve of MDA-MB-468 cell viability following 48-hour treatment with Citropten alone at different concentrations (blue line) versus citropten combined with 11-Cl-BBQ at concentrations of 1, 10 and 50 nM. B) Dose-response curve of MDA-MB-468 cell viability following 48-hour treatment with 11-Cl-BBQ alone (blue line) or 11-Cl-BBQ combined with 10 μM Citropten.

FIGS. 75A-75C compare the effects of 11-Cl-BBQ and Evoxine in hepatoma: A) Mouse Hepa-1 cells were treated with 11-Cl-BBQ, Evoxine either alone or in combination for 48 hours before assessing cell viability using Cell-Titer Glo Viability Assay. B) Hepa-1 cells treated with Evoxine, 11-Cl-BBQ either alone or in combination for 48 hours with induction of cell death measured by Annexin-V staining and detection by flow cytometry. C) Colony forming assay of HepG2 human hepatocellular carcinoma cells seeded at 300 cell/well in a 24-well plate before treatment with 11-Cl-BBQ, Evoxine either alone or in combination at the indicated concentrations for two weeks, followed by fixation and staining with methylene blue. Quantitation of colonies is shown on the right.

FIG. 76 presents representative AhR compounds (BBQ analogs) of the invention effective for activation of AhR transcription. Activation values greater than 1 at 100 nM were considered a hit with nanomolar affinity to promote AhR activity and synergize with the identified compounds from the synergy screens. Referring to FIG. 76, Fold@ 1 nM refers to activation of AhR transcription at 1 nM; Fold@100 nM refers to activation of AbR transcription at 100 nM; and Fold rel @ 100 refers to relative activation at 100 nM compared to parental compound, 11-Cl-BBQ. The representative AhR ligand compounds presented in FIG. 76 activate AhR transcription and therefore are useful as anti-cancer agents, either alone or in combination with the synergizing compounds (and chemotherapeutic agents) described herein. Atypical AhR ligands, such as PRXS 848 and 849, did not activate AhR but induced AhR-selective anti-cancer effects.

FIGS. 77A and 77B show the synergistic effects of Evoxine and Citropten with CGS15943, respectively: MDA-MB-468 cells were treated with CGS15943 (100, 300 nano molar (nM)), Evoxine (10 micro molar (uM)) and Citropten (10 micro molar) either alone or in combination for 40 hours. Cells were harvested and analyzed by Flow cytometry for Apoptosis by Annexin V staining.

### DETAILED DESCRIPTION

### I. Abbreviations and Definitions

The following explanations of terms and abbreviations are provided to better describe the present disclosure and to guide those of ordinary skill in the art in the practice of the present disclosure.

Unless explained otherwise, all technical and scientific terms used herein have the same meaning as commonly understood to one of ordinary skill in the art to which this disclosure pertains. Although methods and materials similar or equivalent to those described herein can be used to practice or test the present disclosure, suitable methods and materials are described below. The materials, methods, and examples are illustrative only and are not intended to be limiting. Other features of the disclosure are apparent from the following detailed description and the claims.

The disclosure of numerical ranges should be understood to refer to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art.

When directly and explicitly distinguishing embodiments from discussed prior art, the embodiment numbers are not approximates unless the word “about” is recited. Definitions of common terms in molecular biology may be found in Benjamin Lewin, Genes VII, published by Oxford University Press, 2000 (ISBN 019879276X); Kendrew et al. (eds.), The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar references.

Adjuvant: Refers to an excipient that modifies the effect of other agents, typically the active ingredient. Adjuvants are often pharmacological and/or immunological agents. An adjuvant may modify the effect of an active ingredient by increasing an immune response. An adjuvant may also act as a stabilizing agent for a formulation. Exemplary adjuvants include, but are not limited to, aluminum hydroxide, alum, aluminum phosphate, killed bacteria, squalene, detergents, cytokines, paraffin oil, and combination adjuvants, such as Freund's complete adjuvant or Freund's incomplete adjuvant.

Agonist: A compound that binds to a receptor or an enzyme and produces an action. For example, an agonist that binds to a cellular receptor initiates a physiological or pharmacological response characteristic of that receptor. An agonist that binds to an enzyme activates the enzyme. An antagonist blocks an action of an agonist.

Alkyl: A hydrocarbon group having a saturated carbon chain. The chain may be cyclic, branched or unbranched. Examples, without limitation, of alkyl groups include methyl, ethyl, propyl, butyl, pentyl, hexyl, heptyl, octyl, nonyl and decyl. The term lower alkyl means the chain includes 1-10 carbon atoms.

The terms alkenyl and alkynyl refer to hydrocarbon groups having carbon chains containing one or more double or triple bonds, respectively.

Analog, Derivative or Mimetic: An analog is a molecule that differs in chemical structure from a parent compound, for example a homolog (differing by an increment in the chemical structure, such as a difference in the length of an alkyl chain), a molecular fragment, a structure that differs by one or more functional groups, a change in ionization. Structural analogs are often found using quantitative structure activity relationships (QSAR), with techniques such as those disclosed in Remington (The Science and Practice of Pharmacology, 19th Edition (1995), chapter 28). A derivative is a biologically active molecule derived from the base structure. A mimetic is a molecule that mimics the activity of another molecule, such as a biologically active molecule. Biologically active molecules can include chemical structures that mimic the biological activities of a compound.

Aromatic or aryl: Unsaturated, cyclic hydrocarbons having alternate single and double bonds. Benzene, a 6-carbon ring containing three double bonds, is a typical aromatic compound.

Carbonyl: A radical of the formula —C(O)—. Carbonyl-containing groups include any substituent containing a carbon-oxygen double bond (C═O), including acyl groups, amides, carboxy groups, esters, ureas, carbamates, carbonates and ketones and aldehydes, such as substituents based on —COR or —RCHO where R is an aliphatic, heteroaliphatic, alkyl, heteroalkyl, hydroxyl, or a secondary, tertiary, or quaternary amine.

Combination: A combination comprises two or more components that are administered such that the effective time period of the first component overlaps with the effective time period of the second and subsequent components. A combination may be a composition comprising the components, or it may be two or more individual components administered substantially simultaneously, or sequentially in any order.

Cyclic: Designates a substantially hydrocarbon, closed-ring compound, or a radical thereof. Cyclic compounds or substituents also can include one or more sites of unsaturation, but does not include aromatic compounds. One example of such a cyclic compound is cyclopentadienone.

Effective amount: With reference to a compound or composition, means an amount of the compound or composition sufficient to achieve a particular desired result, such as to inhibit a protein or enzyme; to elicit a desired biological or medical response in a tissue, system, subject or patient; to treat a specified disorder or disease; to ameliorate or eradicate one or more of its symptoms; and/or to prevent the occurrence of the disease or disorder. The amount of a compound which constitutes an “effective amount” may vary depending on the compound, the desired result, the disease state and its severity, the age of the patient to be treated, and the like.

Excipient: A substance that is used as an additive in a pharmaceutical composition. An excipient can be used, for example, to dilute an active agent and/or to modify properties of a pharmaceutical composition. Examples of excipients include but are not limited to polyvinylpyrrolidone (PVP), tocopheryl polyethylene glycol 1000 succinate (also known as vitamin E TPGS, or TPGS), dipalmitoyl phosphatidyl choline (DPPC), trehalose, sodium bicarbonate, glycine, sodium citrate, and lactose.

Heteroaryl: An aromatic compound or group having at least one heteroatom, i.e., one or more carbon atoms in the ring has been replaced with an atom having at least one lone pair of electrons, typically nitrogen, oxygen, phosphorus, silicon, or sulfur.

Heterocyclic: Refers to a closed-ring compound, or radical thereof as a substituent bonded to another group, particularly other organic groups, where at least one atom in the ring structure is other than carbon, and typically is oxygen, sulfur and/or nitrogen.

Isomer: One of two or more molecules having the same number and kind of atoms, but differing in the arrangement or configuration of the atoms. Isomers that differ in the arrangement of their atoms in space are termed “stereoisomers”. Stereoisomers that are not mirror images of one another are termed “diastereomers” and those that are non-superimposable mirror images of each other are termed “enantiomers.” When a compound has an asymmetric center, for example, if a carbon atom is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−) isomers respectively). A chiral compound can exist as either an individual enantiomer or as a mixture thereof. A mixture containing equal proportions of the enantiomers is called a “racemic mixture.”

Ligand: A molecule that binds to a receptor, having a biological effect.

Pharmaceutically acceptable: A substance that can be taken into a subject without significant adverse toxicological effects on the subject. The term “pharmaceutically acceptable form” means any pharmaceutically acceptable derivative or variation, such as stereoisomers, stereoisomer mixtures, enantiomers, solvates, hydrates, isomorphs, polymorphs, pseudomorphs, neutral forms, salt forms, and prodrug agents.

Pharmaceutically acceptable carrier: Remington: The Science and Practice of Pharmacy, The University of the Sciences in Philadelphia, Editor, Lippincott, Williams, & Wilkins, Philadelphia, PA, 21^{st} Edition (2005), describes compositions and formulations suitable for pharmaceutical delivery of one or more therapeutic compositions and additional pharmaceutical agents. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In some examples, the pharmaceutically acceptable carrier may be sterile to be suitable for administration to a subject (for example, by parenteral, intramuscular, or subcutaneous injection). In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. In some examples, the pharmaceutically acceptable carrier is a non-naturally occurring or synthetic carrier. The carrier also can be formulated in a unit-dosage form that carries a preselected therapeutic dosage of the active agent, for example in a pill, vial, bottle, or syringe.

Pharmaceutically acceptable excipient: A substance, other than the active ingredient, that is included in a formulation of the active ingredient. Excipients can include, but are not limited to, anti-adherents, binders, coatings, enteric coatings, disintegrants, flavorings, sweeteners, colorants, lubricants, glidants, sorbents, preservatives, adjuvants, carriers or vehicles. Excipients may be starches and modified starches, cellulose and cellulose derivatives, saccharides and their derivatives such as disaccharides, polysaccharides and sugar alcohols, protein, synthetic polymers, crosslinked polymers, antioxidants, amino acids or preservatives. Exemplary excipients include, but are not limited to, magnesium stearate, stearic acid, vegetable stearin, sucrose, lactose, starches, hydroxypropyl cellulose, hydroxypropyl methylcellulose, xylitol, sorbitol, maltitol, gelatin, polyvinylpyrrolidone (PVP), polyethyleneglycol (PEG), tocopheryl polyethylene glycol 1000 succinate (also known as vitamin E TPGS, or TPGS), carboxy methyl cellulose, dipalmitoyl phosphatidyl choline (DPPC), vitamin A, vitamin E, vitamin C, retinyl palmitate, selenium, cysteine, methionine, citric acid, sodium citrate, methyl paraben, propyl paraben, sugar, silica, talc, magnesium carbonate, sodium starch glycolate, tartrazine, aspartame, benzalkonium chloride, sesame oil, propyl gallate, sodium metabisulphite or lanolin.

Pharmaceutically acceptable salt: A biologically compatible salt of a compound that can be used as a drug, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate, and the like. Pharmaceutically acceptable acid addition salts are those salts that retain the biological effectiveness of the free bases while formed by acid partners that are not biologically or otherwise undesirable, e.g., inorganic acids such as hydrochloric acid, hydrobromic acid, sulfuric acid, nitric acid, phosphoric acid, and the like, as well as organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, benzene sulfonic acid (besylate), cinnamic acid, mandelic acid, methanesulfonic acid, ethanesulfonic acid, p-toluenesulfonic acid, salicylic acid and the like. Pharmaceutically acceptable base addition salts include those derived from inorganic bases such as sodium, potassium, lithium, ammonium, calcium, magnesium, iron, zinc, copper, manganese, aluminum salts and the like. Exemplary salts are ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. (See, for example, S. M. Berge, et al., “Pharmaceutical Salts,” J. Pharm. Sci., 1977; 66:1-19, which is incorporated herein by reference.)

Prodrug: Compounds that are transformed in vivo to yield a biologically active compound, particularly the parent compound, for example, by hydrolysis in the gut or enzymatic conversion. Common examples of prodrug moieties include, but are not limited to, ester and amide forms of a compound having an active form bearing a carboxylic acid moiety. Prodrugs are familiar to a person of ordinary skill in the art, as indicated by a thorough discussion of prodrugs by T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987.

Senescence: Biological aging, gradual deterioration of functional characteristics in living organisms. Senescent means to be characterized by senescence, to be deteriorating.

Senolytic: As used herein, the term “senolytic” refers to a small molecule capable of selectively inducing death of senescent cells.

Solvate: A complex formed by combination of solvent molecules with molecules or ions of a solute. The solvent can be an organic solvent, an inorganic solvent, or a mixture of both. Exemplary solvents include, but are not limited to, alcohols, such as methanol, ethanol, propanol; amides such as N,N-dialiphatic amides, such as N,N-dimethylformamide; tetrahydrofuran; alkylsulfoxides, such as dimethylsulfoxide; water; and combinations thereof. The compounds described herein can exist in un-solvated as well as solvated forms when combined with solvents, pharmaceutically acceptable or not, such as water, ethanol, and the like. Solvated forms of the presently disclosed compounds are within the scope of the embodiments disclosed herein.

Stereochemistry: The three-dimensional spatial configuration of a molecule.

Stereoisomers: Isomers that have the same molecular formula and sequence of bonded atoms, but which differ only in the three-dimensional orientation of the atoms in space.

Subject: An animal (human or non-human) subjected to a treatment, observation or experiment. Includes both human and veterinary subjects, including human and non-human mammals, such as rats, mice, cats, dogs, pigs, horses, cows, and non-human primates.

Substituted: A fundamental compound, such as an aryl or aliphatic compound, or a radical thereof, having coupled thereto one or more substituents, each substituent typically replacing a hydrogen atom on the fundamental compound. A person of ordinary skill in the art will recognize that compounds disclosed herein may be described with reference to particular structures and substituents coupled to such structures, and that such structures and/or substituents also can be further substituted, unless expressly stated otherwise or context dictates otherwise. Solely by way of example and without limitation, a substituted aryl compound may have an aliphatic group coupled to the closed ring of the aryl base, such as with toluene. Again solely by way of example and without limitation, a long-chain hydrocarbon may have a hydroxyl group bonded thereto. Groups which are substituted (e.g., substituted alkyl), may in some embodiments be substituted with a group which is substituted (e.g., substituted aryl). In some embodiments, the number of substituted groups linked together is limited to two (e.g., substituted alkyl is substituted with substituted aryl, wherein the substituent present on the aryl is not further substituted). In some embodiments, a substituted group is not substituted with another substituted group (e.g., substituted alkyl is substituted with unsubstituted aryl).

Substituent: An atom or group of atoms that replaces another atom in a molecule as the result of a reaction. The term “substituent” typically refers to an atom or group of atoms that replaces a hydrogen atom, or two hydrogen atoms if the substituent is attached via a double bond, on a parent hydrocarbon chain or ring. The term “substituent” may also cover groups of atoms having multiple points of attachment to the molecule, e.g., the substituent replaces two or more hydrogen atoms on a parent hydrocarbon chain or ring. In such instances, the substituent, unless otherwise specified, may be attached in any spatial orientation to the parent hydrocarbon chain or ring. Exemplary substituents include, for instance, alkyl, alkenyl, alkynyl, alkoxy, alkylamino, alkylthio, acyl, aldehyde, amido, amino, aminoalkyl, aryl, arylalkyl, arylamino, carbonate, carboxyl, cyano, cycloalkyl, dialkylamino, halo, haloaliphatic (e.g., haloalkyl), haloalkoxy, heteroaliphatic, heteroaryl, heterocycloaliphatic, hydroxyl, oxo, sulfonamide, sulfhydryl, thio, and thioalkoxy groups.

Therapeutically effective amount or dose: An amount sufficient to provide a beneficial, or therapeutic, effect to a subject or a given percentage of subjects.

Therapeutic time window: The length of time during which an effective, or therapeutic dose, of a compound remains therapeutically effective in vivo.

Treating or treatment: Concerns treatment of a disease or condition of interest in a patient or subject, particularly a human having the disease or condition of interest, and includes by way of example, and without limitation:
(i) preventing the disease or condition from occurring in a patient or subject, in particular, when such patient or subject is predisposed to the condition but has not yet been diagnosed as having it;
(ii) inhibiting the disease or condition, for example, arresting or slowing its development;
(iii) relieving the disease or condition, for example, causing regression of the disease or condition or a symptom thereof; or
(iv) stabilizing the disease or condition.

As used herein, the terms “disease” and “condition” can be used interchangeably or can be different in that the particular malady or condition may not have a known causative agent (so that etiology has not yet been determined) and it is therefore not yet recognized as a disease but only as an undesirable condition or syndrome, where a more or less specific set of symptoms have been identified by clinicians.

Tumor: An abnormal growth of cells, which can be benign or malignant. Cancer is a malignant tumor, which is characterized by abnormal or uncontrolled cell growth. Other features often associated with malignancy include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original tumor site and migrate to other parts of the body for example via the bloodstream or lymph system. Thus, a metastatic cancer is a cancer at one or more sites in the body other than the site of origin of the original (primary) cancer from which the metastatic cancer is derived. The amount of a tumor in an individual is the “tumor burden” which can be measured as the number, volume, or weight of the tumor. A tumor that does not metastasize is referred to as “benign.”

The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.”

An “established” or “existing” tumor is an existing tumor that can be discerned by diagnostic tests. In some embodiments, an established tumor can be palpated. In some embodiments, an “established tumor” is at least 500 mm³, such as at least 600 mm³, at least 700 mm³, or at least 800 mm³ in size. In other embodiments, the tumor is at least 1 cm long. With regard to a solid tumor, an established tumor generally has a robust blood supply, and has induced Tregs and myeloid derived suppressor cells (MDSCs).

### II. Method of Suppressing Cancer Cell Growth/Inhibiting Tumor Progression

### A. AhR as a Molecular Target for Cancer Drug Development

The fate of cancer cells can be influenced via AhR signaling. AbR therefore has been suggested as a molecular target for cancer drug development. Significant efforts have been focused on identifying AhR ligands with potent anticancer effects, and such ligands are referred to herein as Select Modulators of AhR-regulated Transcription (SMAhRTs).

Initial efforts that led to the presently disclosed embodiments focused on repurposing of FDA-approved drugs with well-characterized toxicity profiles in humans that could function through the AhR for treatment of cancer. This led to identifying leflunomide, flutamide, and raloxifene that suppressed the growth of cancer cells by inducing cell cycle arrest or cell death Additional searching for SMAhRTs involved screening more than 50,000 small molecules from the DIVERSet® library from ChemBridge. A group of benzimidazoisoquinolines, particularly benzimidazobenzoisoquinolinones, such as 10- or 11-Cl-BBQ and its analogs, were discovered to be high affinity AbR ligands. These benzimidazoisoquinolines were shown to activate AhR at nanomolar concentrations, promote T-cell differentiation, and were non-toxic in mouse models.

The anticancer effects of 11-Cl-BBQ in both small and non-small lung cancer cell lines have been determined. 11-Cl-BBQ induced potent anti-proliferative effects in lung cancer cells expressing high levels of the AhR, but not in cells with very low or no expression. Stable knockdown of AhR expression using CRISPR-cas9 in responsive cancer cell lines reversed the anti-cancer effects of 11-Cl-BBQ, indicating that these effects are AbR-dependent. In addition, 11-Cl-BBQ induced a stable G-1 phase cell cycle arrest, and the arrested cells were unable to grow in zebrafish tumor xenograft assays. Transcriptomic studies revealed that p53 signaling is induced and significantly contributed to the cell cycle arrest induced by 11-Cl-BBQ. In addition, these transcriptomic studies also revealed upregulation of three cyclin-dependent kinase inhibitors, p27^{Kip1}, p21^{Cip1} and CABLES1. Knockdown experiments confirmed a role for p27^{Kip1} in AhR-dependent inhibition of proliferation. Overall, these results increased the understanding of the growth suppression mechanisms by the AhR and support development of 11-Cl-BBQ and other related benzimidazoisoquinolines as anti-lung cancer therapeutics. 10-Cl-BBQ and benzimidazoisoquinolines were originally identified as AhR ligands by the present inventors.

### B. 11-Cl-BBQ Inhibits Cancer Cell Growth in an AhR-Dependent Manner

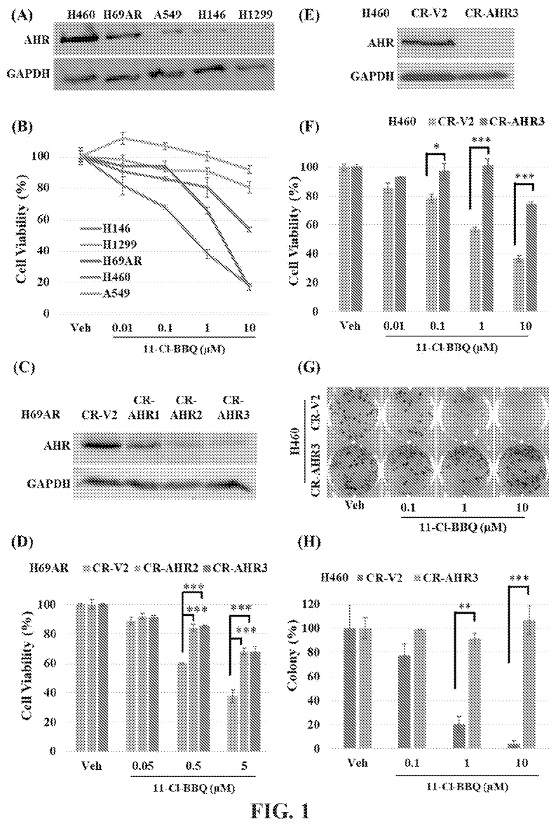

The growth inhibition effect of 11-Cl-BBQ on multiple lung cancer cell lines with varying levels of steady-state AhR expression (FIG. 1A) was specifically investigated. 11-Cl-BBQ decreased the growth of lung cancer cells in a dose-dependent manner from 10 nM to 10 μM concentrations (FIG. 1B). H460 and H69AR cell lines with relatively high expression of AhR were more sensitive to 11-Cl-BBQ than A549 and H1299 cells, each with relatively low expression of the AhR (FIGS. 1A and 1B), suggesting a role for the AhR in the growth suppression induced by 11-Cl-BBQ. To determine whether the AhR was required for 11-Cl-BBQ-induced growth suppression, stable AhR-deficient cells were generated that were derived from H69AR lung cancer cells utilizing CRIPSR-cas9 with three single guide RNAs (sgRNAs; CR-AHR1, CR-AHR2, and CR-AHR3) targeting the human AhR gene. Western blot analysis demonstrated reduction in steady-state levels of AhR expression in pooled cultures following expression of two AhR-specific sgRNAs (CR-AhR2 and CR-AhR3) compared to the control vector (CR-V2) (FIG. 1C). Suppression of AhR expression in H69AR cells significantly reversed the growth inhibitory effects of 11-Cl-BBQ (FIG. 1D). Thus, AhR is required for the growth inhibition induced by 11-Cl-BBQ in H69AR lung cancer cells.

To investigate the role of AhR in additional lung cancer cells, AhR knockout H460 cells were generated utilizing CRIPSR-cas9. DNA-sequencing analysis of a clonal line (CR-AHR3) identified an 86 bp insertion into exon 5 of the AhR gene causing a frameshift and multiple predicted premature stop codons that associated with a dramatic reduction of AhR mRNA and an AbR-knockout phenotype (FIG. 1E). H460 AhR-knockout cells were significantly less responsive to 11-Cl-BBQ treatment compared to the AhR-expressing CR-V2 control cells (FIG. 1F). In addition, continuous treatment with 11-Cl-BBQ for 14 days with 0.1-10 UM concentrations strongly inhibited colony formation of AhR-expressing H460 cells, but not the AhR-deficient H460 cells (FIGS. 1G and 1H). These data establish that AbR mediates growth suppression of 11-Cl-BBQ in two distinct lung cancer cell lines.

### C. 11-Cl-BBQ Induces an Irreversible, AhR-Dependent Cell Cycle Arrest in Lung Cancer Cells

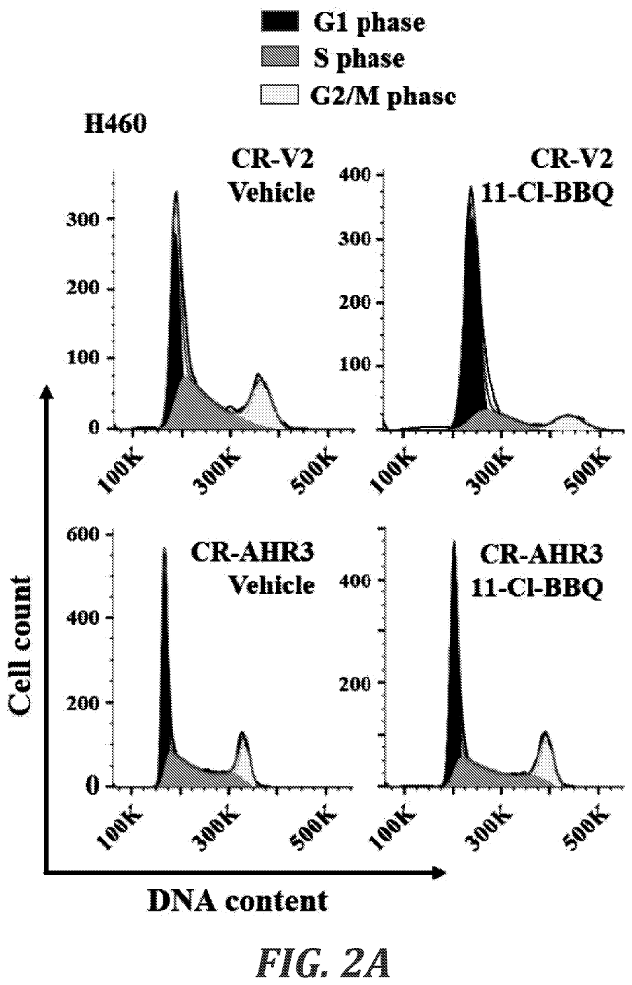

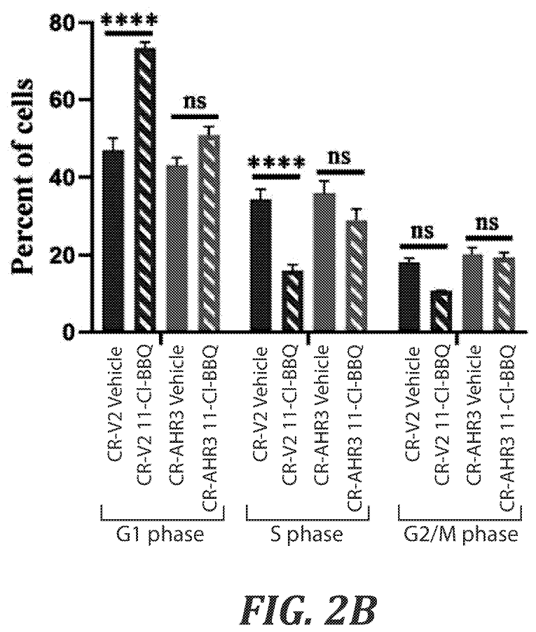

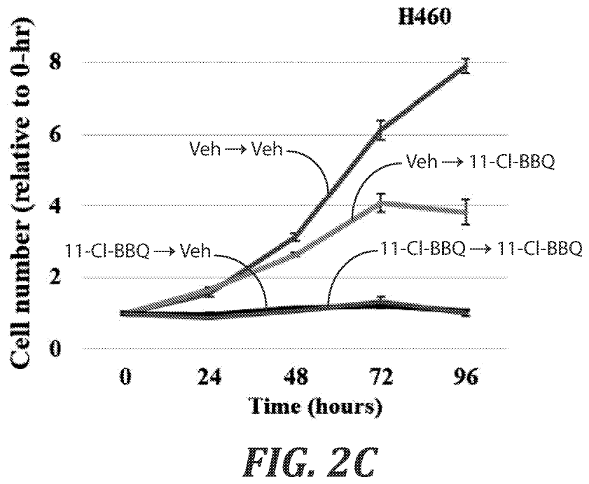

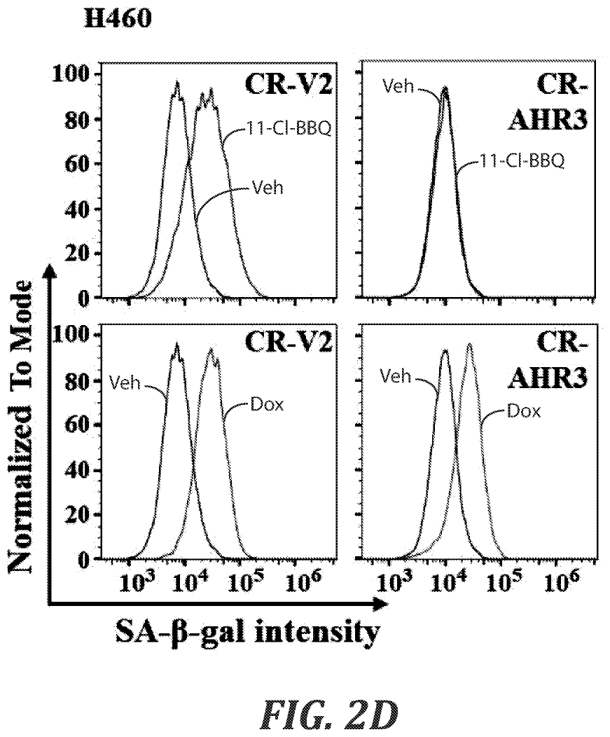

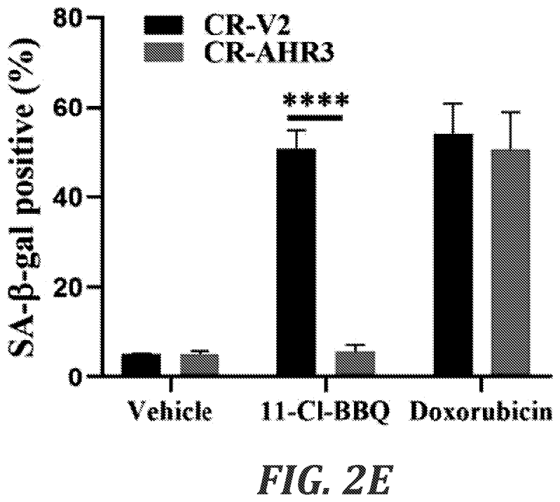

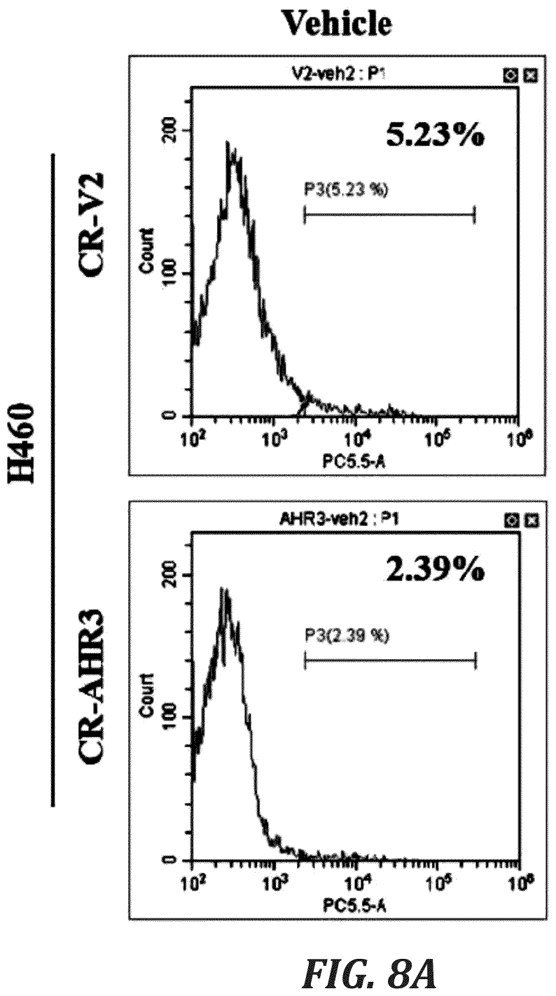

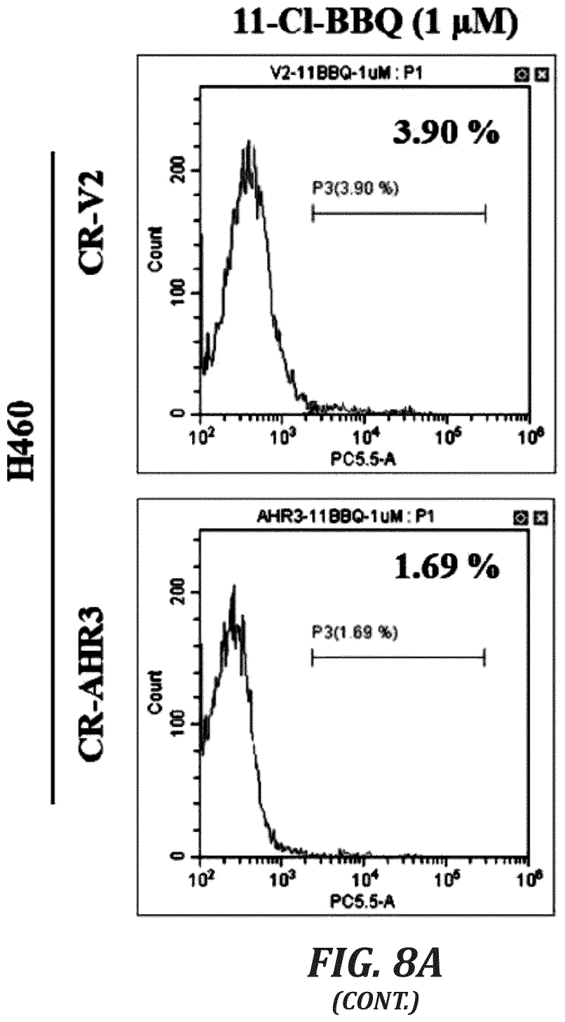

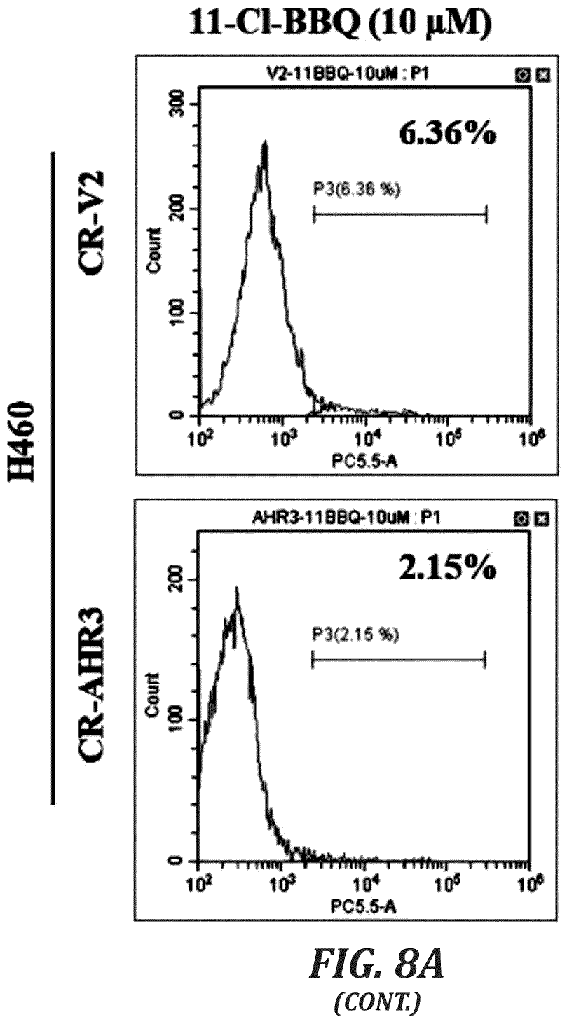

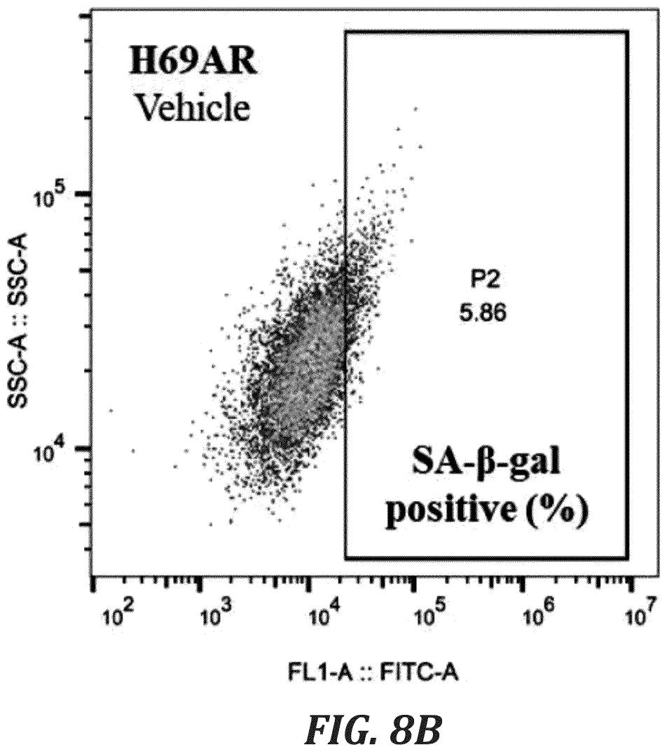

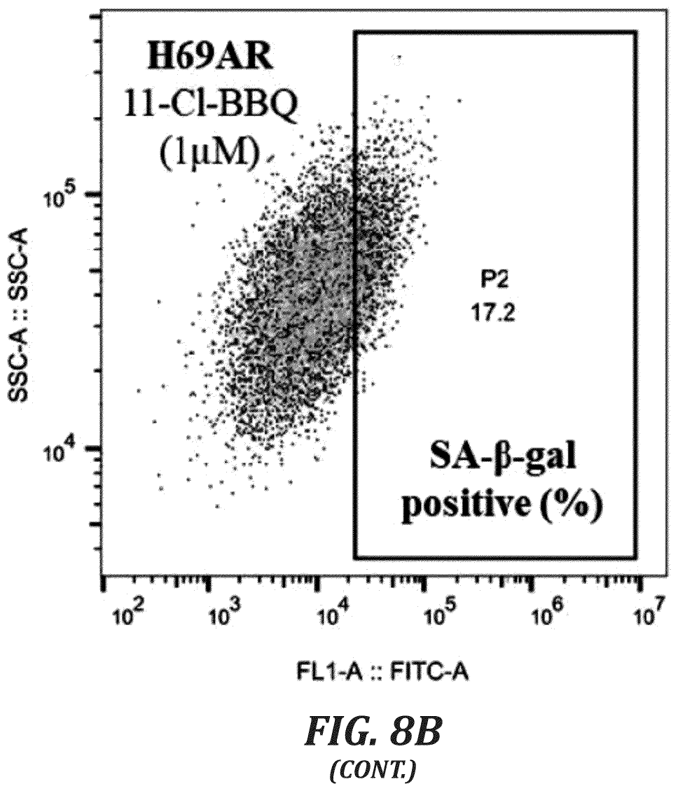

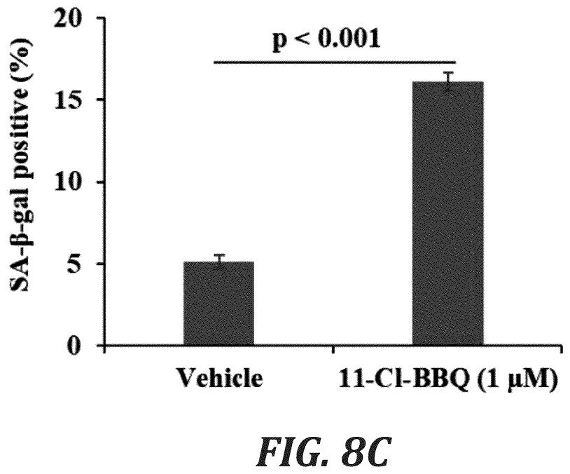

Visual inspection of the lung cancer cells using phase contrast light microscopy suggested that treatment with 11-Cl-BBQ did not induce cell death. In addition, H460 cells treated with 11-Cl-BBQ were negative for Annexin V staining (FIG. 8A), indicating that the growth inhibitory effect of 11-Cl-BBQ was not due to induction of cell death. The effect of 11-Cl-BBQ on H460 cell proliferation was therefore tested by analyzing the cell cycle distribution. There was a significant increase of cells in G1 phase (45% to >70%) and a reduction of cells in S-phase (35% to 15%) upon treatment with 2.5 μM 11-Cl-BBQ for 24 hours (FIGS. 2A and 2B). The G1 phase cell cycle arrest induced by 11-Cl-BBQ was AhR-dependent as this was not observed in H460 cells without the AhR expression (FIGS. 2A and 2B). To determine whether the effect of 11-Cl-BBQ was reversible, AhR-expressing H460 cells were treated with 11-Cl-BBQ or DMSO (vehicle control) for 72 hours, followed by a “washout” period for five days. Continuous exposure to 11-Cl-BBQ (2.5 μM) resulted in sustained growth suppression (FIG. 2C, red line), as expected. However, H460 cells did not recover from growth arrest even after 96 hours of 11-Cl-BBQ wash out period (FIG. 2C, black line). Thus, an investigation was conducted to determine whether 11-Cl-BBQ-induced growth arrest was associated with an increase in the senescence-associated beta galactosidase (SA-β-gal), a well-characterized biomarker for an irreversible cell cycle arrest or senescent phenotype. Indeed, 11-Cl-BBQ treatment (2.5 μM for 120 h) resulted in significant increase in SA-β-gal expression and was AhR-dependent (FIGS. 2D and 2E; compare AhR-proficient CR-V2 versus AhR-deficient CR-AHR3 cells). In contrast, treatment with doxorubicin, a topoisomerase II inhibitor, induced SA-β-gal expression independent of the AhR status in H460 cells (FIGS. 2D and 2E). 11-Cl-BBQ treatment (1 μM for 120 h) also increased SA-β-gal in the H69AR lung cancer cell line (FIGS. 8B, 8C).

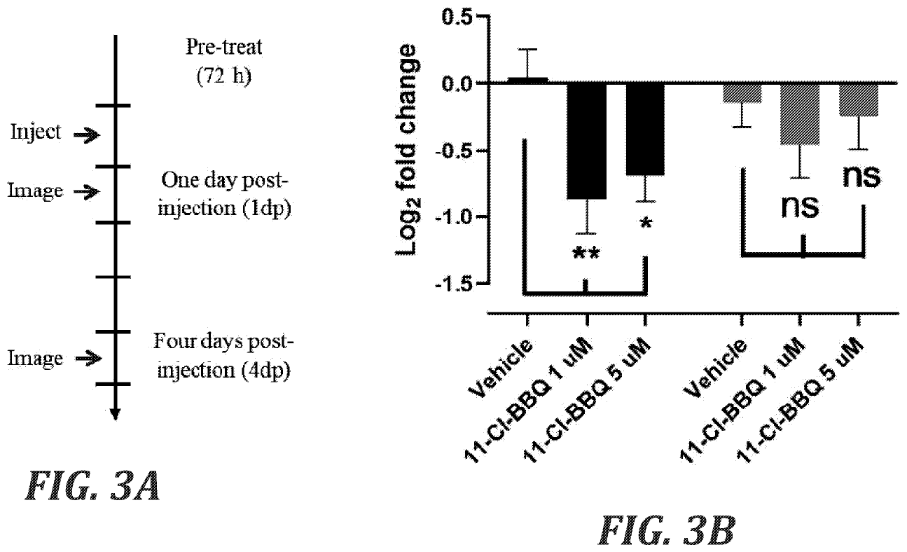

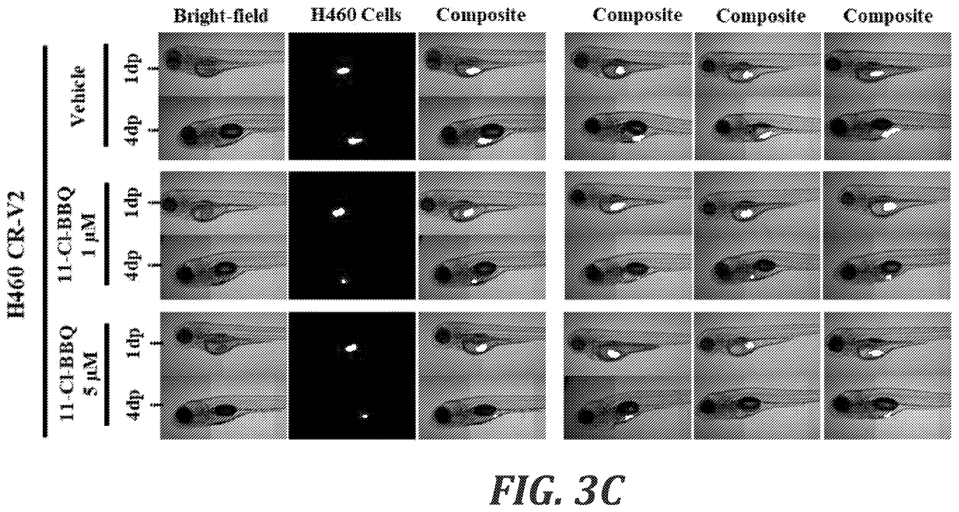

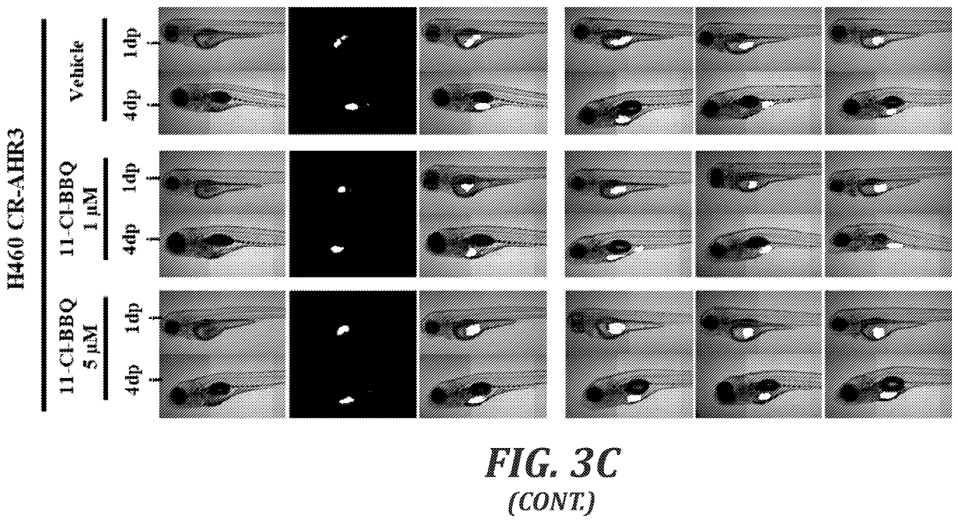

To investigate whether the stable cell cycle arrest induced by 11-Cl-BBQ sustains in an in vivo model, the growth of 11-Cl-BBQ-treated AhR-proficient and AhR-deficient lung cancer cells was monitored in a zebrafish tumor xenograft assay. AhR-expressing (CR-V2) and AhR-deficient (CR-AHR3) H460 cells were exposed to 11-Cl-BBQ (1 μM or 5 μM for 72 hours), after which the compound was washed out. Cells were then injected into the yolk sac of live zebrafish embryos and the growth of the cancer cell xenografts was measured at days 1 and 4 of post-injection (FIG. 3A). As shown in FIGS. 3B and 3C, AhR-expressing 11-Cl-BBQ treated cells had significantly lower growth in this xenograft model. Taken together, the data establishes that 11-Cl-BBQ treatment induces an AhR-dependent and irreversible G1-phase arrest in human lung cancer cells.

### D. 11-Cl-BBQ Suppresses the Expression of Genes Involved in DNA Replication and Cell Cycle Regulation

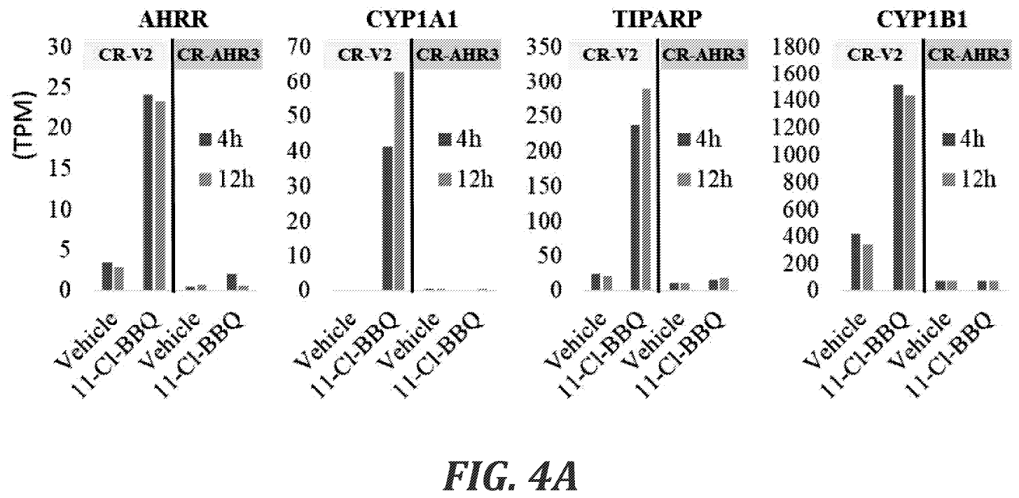

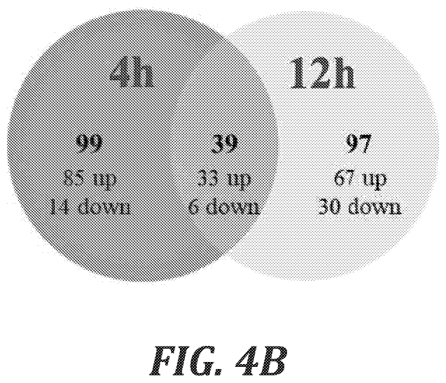

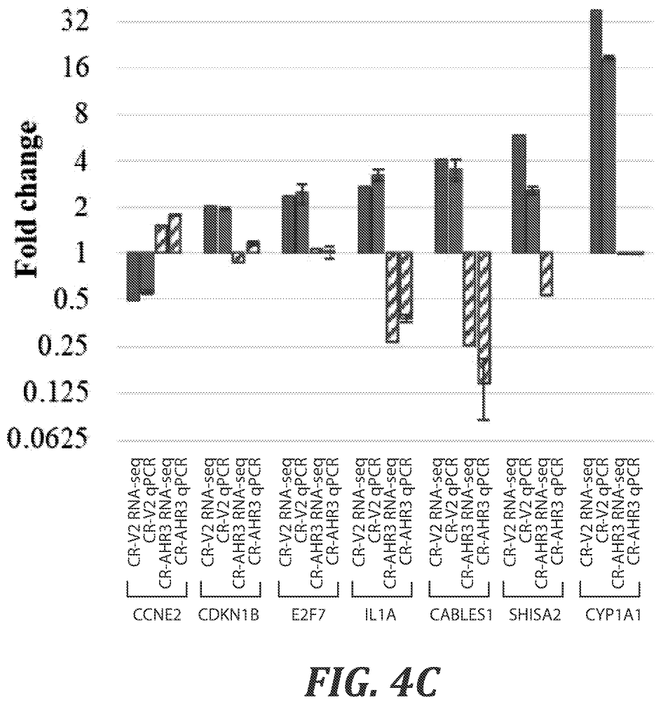

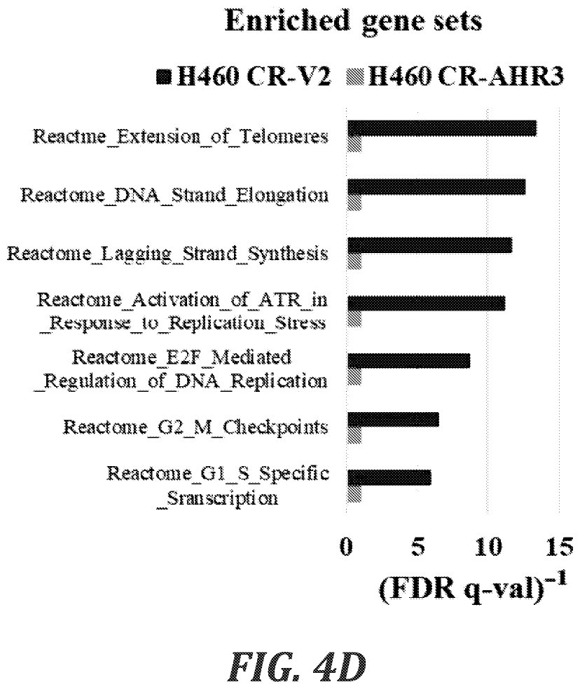

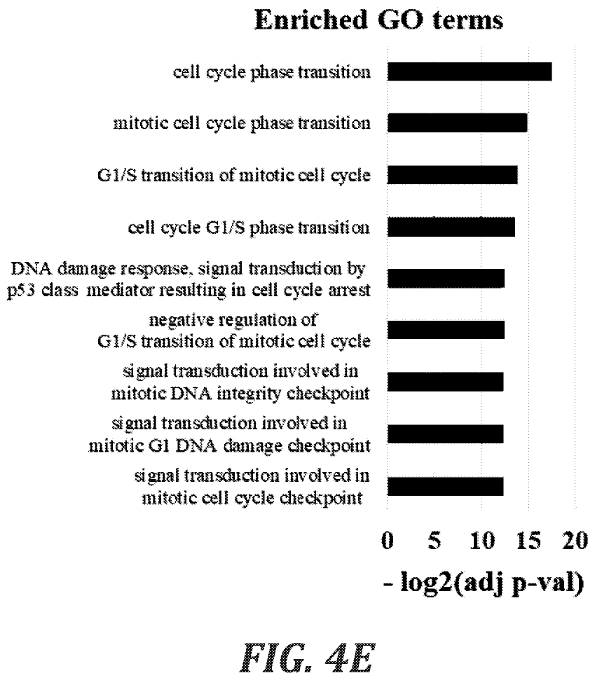

To identify AhR-mediated transcriptional programs relevant to inhibition of lung cancer cell proliferation, changes in global gene expression were measured using RNA-sequencing (RNA-seq). AhR-proficient and -deficient H460 cells were treated with 5 μM 11-Cl-BBQ or vehicle control for 4 hours or 12 hours and analyzed changes in gene expression by RNA-seq. Treatment with 11-Cl-BBQ resulted in significant changes in expression of a number of genes in an AhR-dependent manner (FIGS. 4A and 4B), including the elevation of well-known AhR targets: CYP1A1, CYP1B1, AhRR, and TIPARP (FIG. 4A). Expression changes of at least 2-fold for genes detected with seven or greater Transcripts Per kilobase Million (TPM) were considered significant. Some genes were differentially expressed both at 4 hours and 12 hours (33 elevated and 6 reduced), but several of differentially expressed genes were not the same between these two time points (85/67 elevated and 14/30 reduced at 4/12 hours, respectively) (FIG. 4B). Changes in mRNA levels of previously known AhR-target genes (CYP1A1, ILIA, CDKN1B (p27^{Kip1})) and several newly identified genes (CABLES1, CCNE2, and SHISA2) were confirmed by qPCR (FIG. 4C).

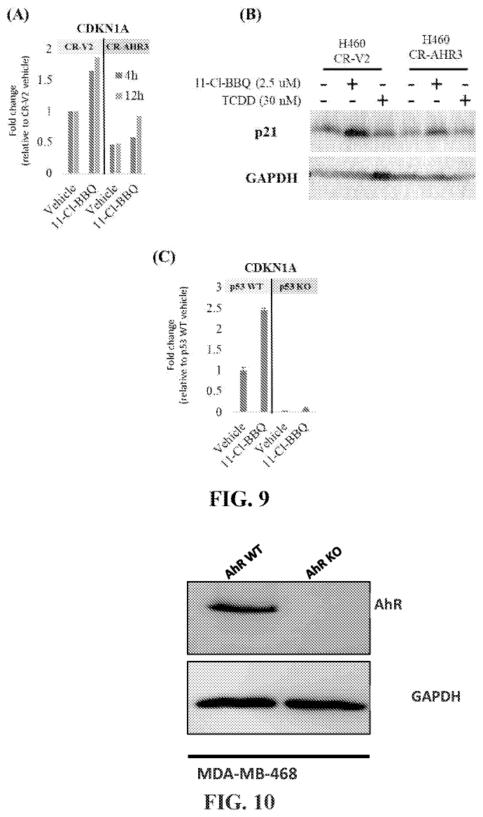

Biological processes often involve multiple genes or a network of genes working in a coordinated manner in the same or closely related pathways. Gene set enrichment analysis (GSEA) was performed using single sample GSEA projection. Several negative regulators of cell cycle progression (CDK inhibitors p27^{Kip1} and CABLES1 (FIG. 4C) and p21^{Cip1} (FIGS. 9A and 9B) were elevated in an AhR-dependent manner after 4 h of 11-Cl-BBQ treatment. GSEA further revealed enrichment of low expressed genes (FDR<25%) in canonical pathways associated with DNA replication and cell cycle progression in AhR-expressing (but not in AhR-deficient) H460 lung cancer cells after 12 hours exposure to 11-Cl-BBQ (FIG. 4D). These results are consistent with the induction of G1 cell cycle arrest by 11-Cl-BBQ (FIG. 2).

The RNA-seq data set includes paired-end, long reads (150 bps) which allows for transcript-level quantification. A transcript-level quantification of the RNA-seq data was generated as an alternate approach to identify changes in gene expression induced by 11-Cl-BBQ in lung cancer cells. This led to the identification of 777 transcripts altered in an AhR-dependent manner by 2-fold or more after 12 hours of 11-Cl-BBQ treatment. Gene ontology (GO) enrichment of the transcripts was analyzed using DAVID functional annotation clustering tool. The results revealed a cluster of functions related to cell cycle regulation (Cluster 4, enrichment score 5.04; FIG. 4E) as one of the top enriched clusters. Consistent with the gene-level GSEA results (FIG. 4D), the top 9 most enriched functional annotation terms in the cluster 4 were associated with DNA replication, G1/S phase cell cycle progression, and signal transduction associated with cellular responses to DNA damage for both the G1 and M phase checkpoints (FIG. 4E). Among the enriched GO terms was signal transduction by p53 resulting in cell cycle arrest, suggesting the involvement of the tumor suppressor p53 in 11-Cl-BBQ-induced cell cycle arrest. Together, the analyses of cell cycle progression (FIG. 2) and gene expression (FIG. 4) demonstrate that activation of AhR by 11-Cl-BBQ results in an irreversible G1-phase cell cycle arrest in lung cancer cells associated with the activation of signal transduction pathways related to cellular checkpoint responses and the suppression of the expression of genes necessary for DNA replication. This analysis identified additional potential key mediators of 11-Cl-BBQ-induced growth suppression downstream of AhR activation.

### E. Tumor Suppressor PS3 is Required for 11-Cl-BBQ-Induced G1 Phase Cell Cycle Arrest in Lung Cancer Cells

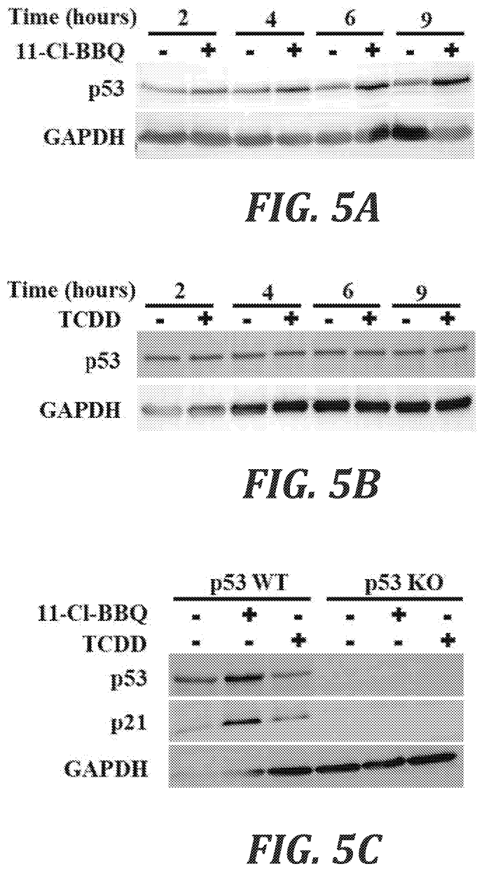

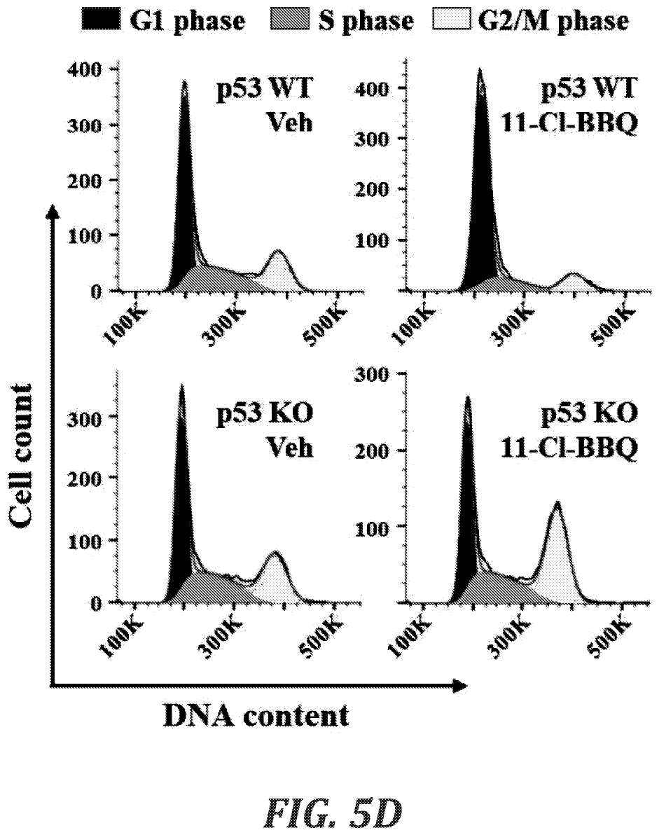

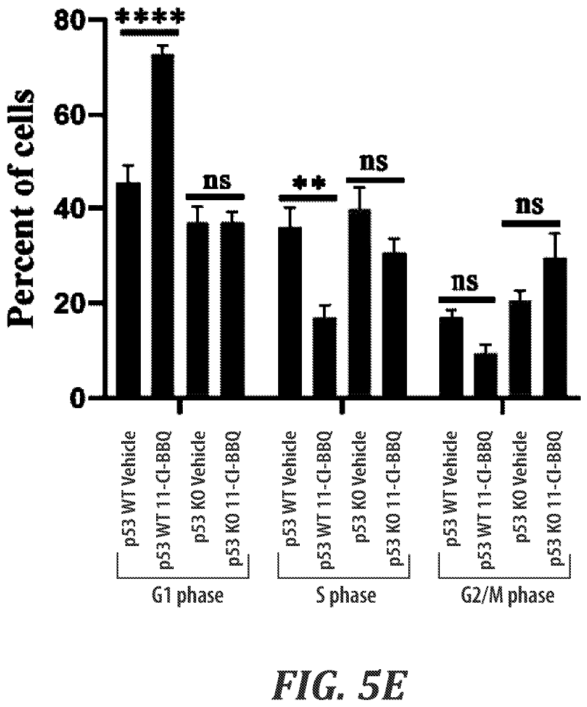

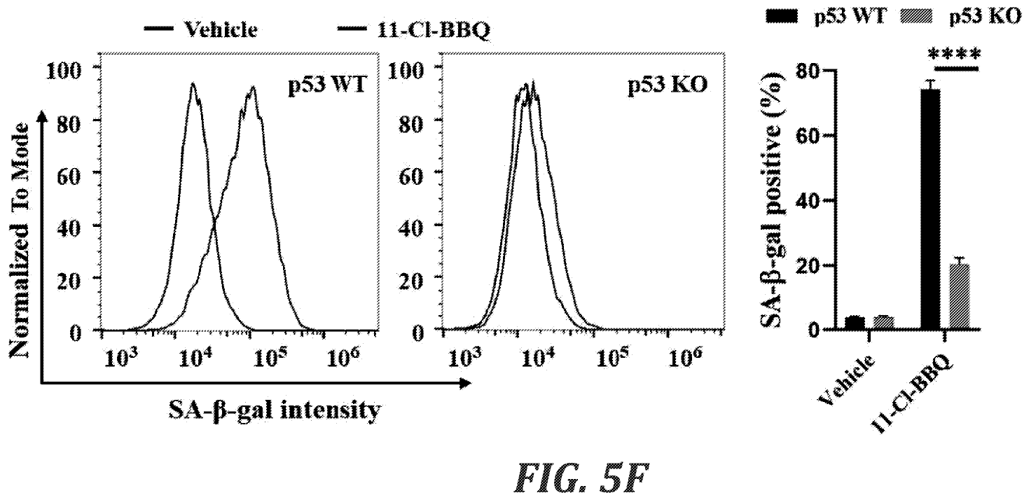

The analysis of gene ontology terms associated with changes of transcripts suggested the involvement of p53-dependent signal transduction in the 11-Cl-BBQ-induced cell cycle arrest in lung cancer cells (FIG. 4E). In addition, transcription of the CDK inhibitor p21^{Cip1}, a well-known downstream target of p53 was up-regulated by 11-Cl-BBQ in H460 lung cancer cells in an AhR-dependent manner (FIGS. 9A and 9B). This prompted an investigation of the role of p53 in the AhR-mediated cell cycle arrest. H460 lung cancer cells express wild type p53 and treatment with 11-Cl-BBQ (5 μM) resulted in increased p53 protein levels starting from 4 h, with further increases after 6 and 9 h (FIGS. 5A and 5C). However, expression of p53 protein was not altered (FIGS. 5B and 5C) by treatment with TCDD, a potent ligand of AhR. To test if p53 has a role in AhR-mediated growth suppression, p53-deficient H460 cells were generated using the CRISPR-cas9 (FIG. 5C). Knockout of p53 lowered the basal expression of p21^{Cip1} and its induction, both at mRNA and protein levels (FIG. 9C and FIG. 5C). H460 p53-proficient cells were arrested in G1 phase upon 11-Cl-BBQ treatment as expected; however, the G1 cell cycle arrest was not detected in p53 knockout H460 cells (FIGS. 5D and SE). However, an increase in G2 phase cells was noticed in p53 knockout cells upon treatment with 11-Cl-BBQ. Knockout of p53 also significantly reduced SA-β-gal, a biomarker for long-term cell cycle arrest (FIG. 5F) upon 11-Cl-BBQ treatment. These data indicate an important role of p53 in AhR-mediated cell cycle arrest.

F. p27^{Kip1} is an Important Downstream Target Gene of the AhR for Suppression of Lung Cancer Cell Proliferation

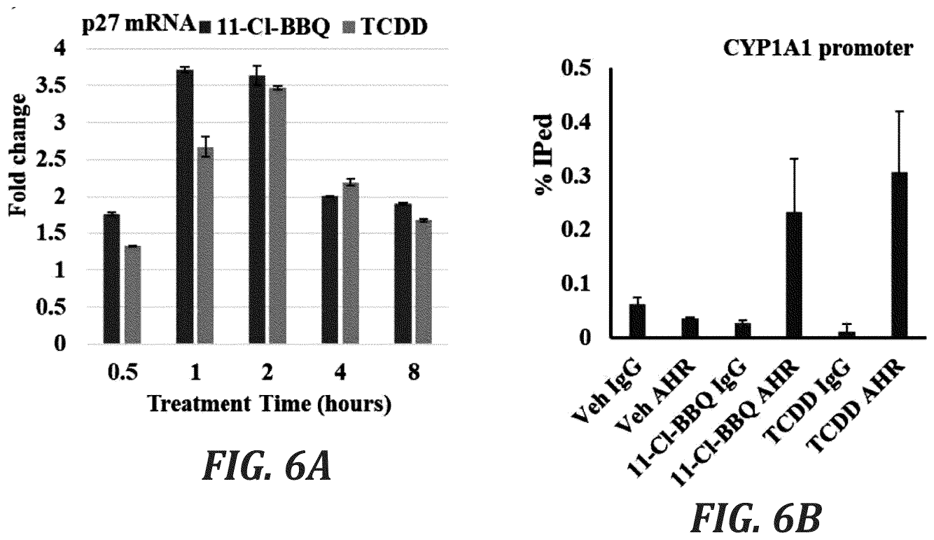

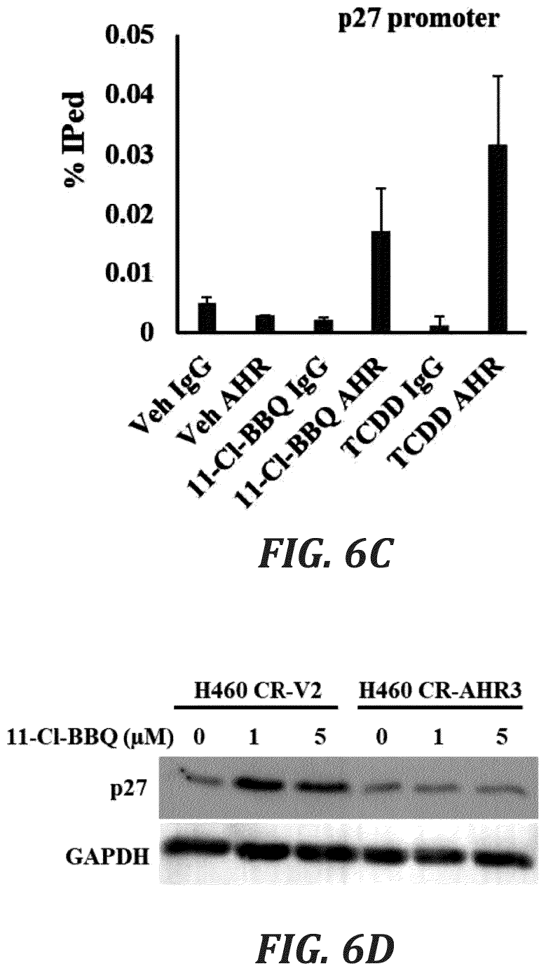

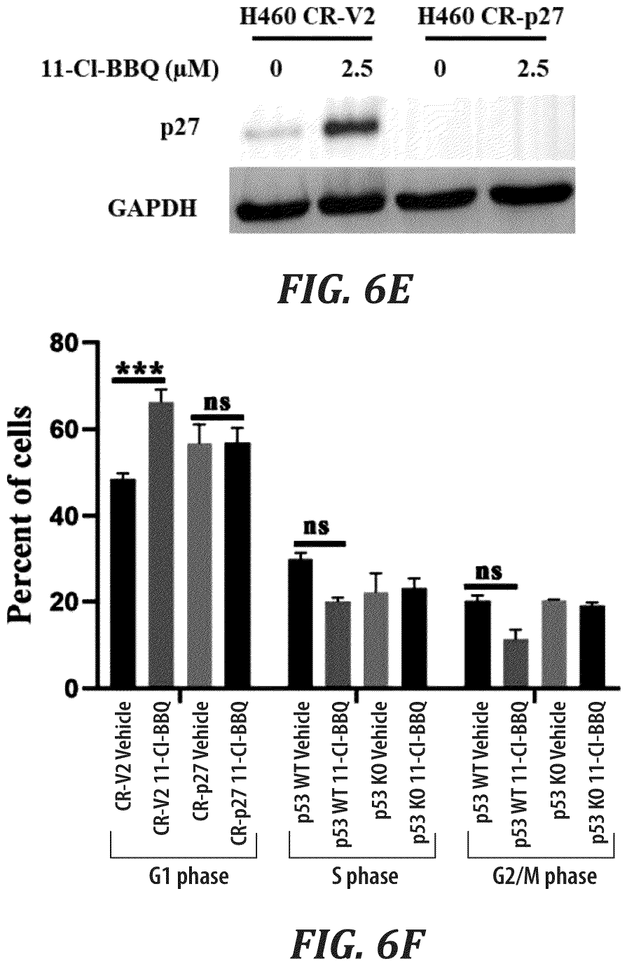

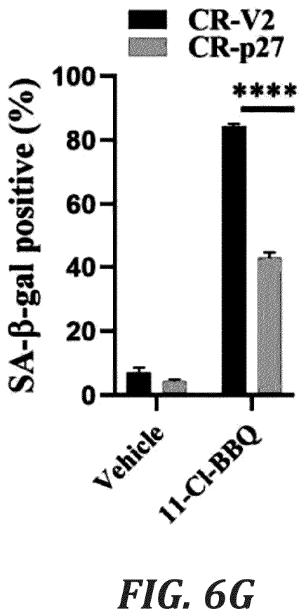

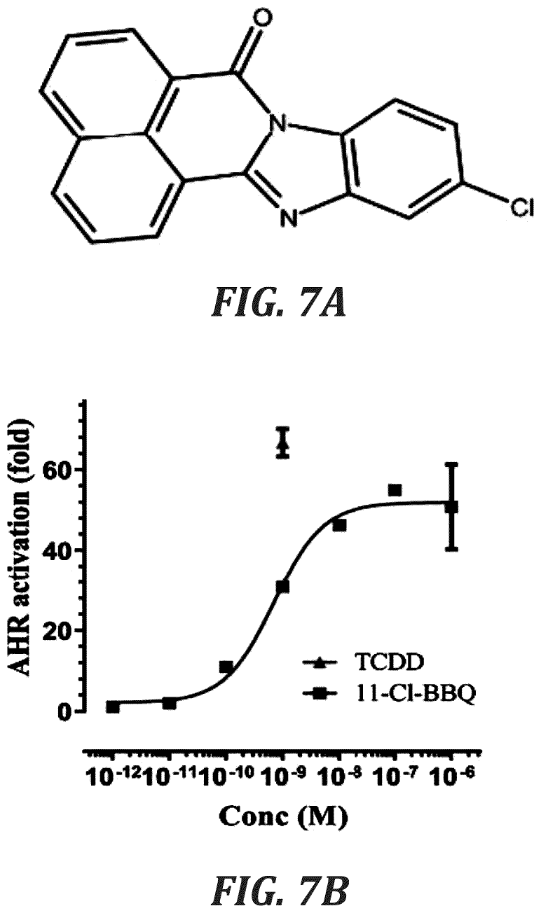

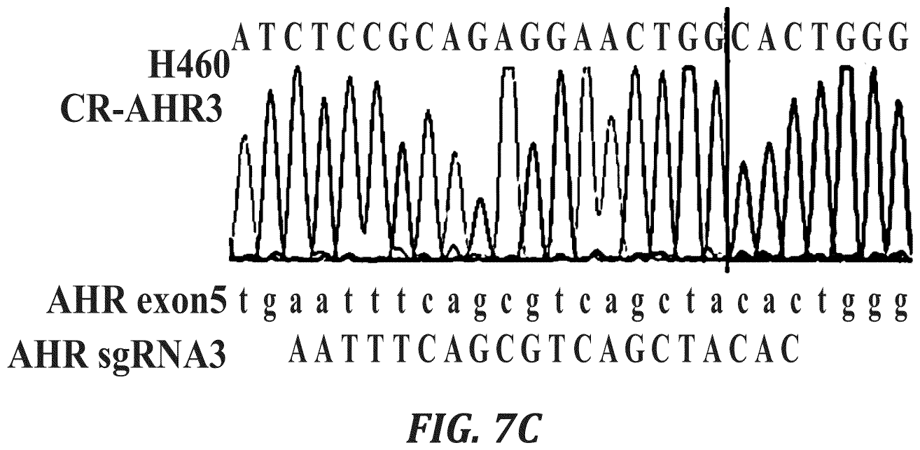

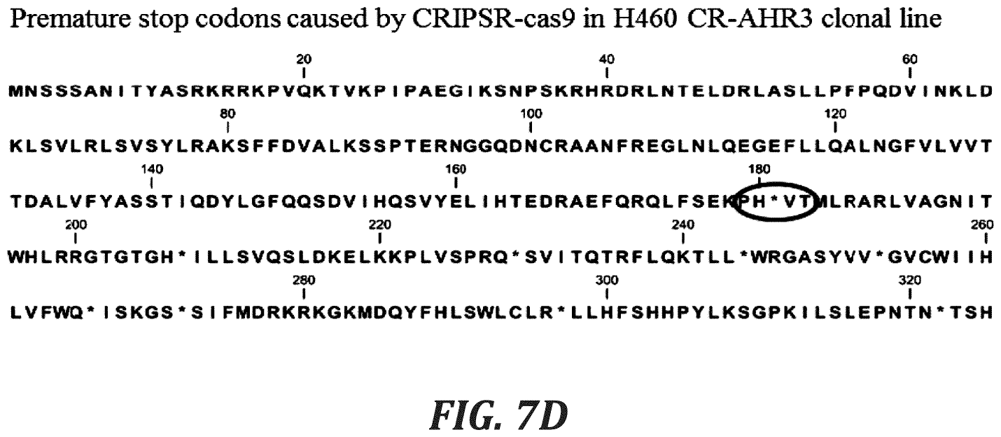

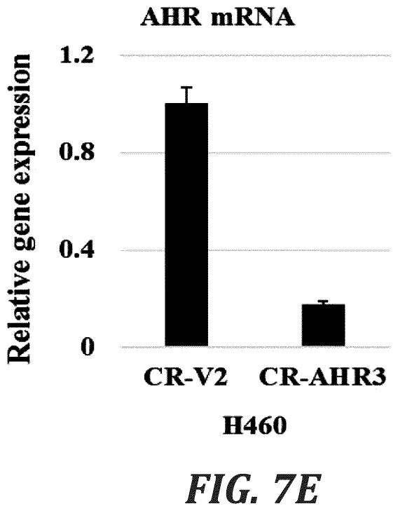

An early induction (4 hours) of CDKN1B (p27^{Kip1}) by 11-CL-BBQ was identified in an AhR-dependent manner during RNA seq analysis (FIG. 4C). The induction of mRNA was confirmed in a time-course experiment (0.5 hour to 8 hours) upon treatment with 11-Cl-BBQ (2.5 μM) by qPCR (FIG. 6A). To investigate whether the AhR regulated p27^{Kip1} directly, chromatin immunoprecipitation (ChIP) was performed using an AhR-specific polyclonal antibody to detect association of the AhR with CDKN1B (p27^{Kip1}) gene promoter. Results of the ChIP assay confirmed recruitment of the AhR to CYP1A1 gene promoter, upon treatment with both 11-Cl-BBQ and TCDD (FIG. 6B). Similarly, the AhR was recruited to the CDKN1B (p27^{Kip1}) gene promoter by both 11-Cl-BBQ and TCDD (FIG. 6C). In addition, a time-course analysis (FIG. 6A) demonstrated that CDKN1B (p27^{Kip1}) is induced by 11-Cl-BBQ as early as 1 h, consistent with the notion that p27^{Kip1} is a direct target of the AhR (FIGS. 6B and 6C). The increased p27^{Kip1} protein levels were seen even after 72 hours of treatment (FIG. 6D).

To determine whether p27^{Kip1} was required for the 11-Cl-BBQ-induced cell cycle arrest, CRISPR-cas9 was used to generate stable p27^{Kip1}-deficient cells from the H460 lung cancer cell line. The knockout of p27^{Kip1} was clearly confirmed at the protein level by Western blot analysis (FIG. 6E). There were higher percentage of G1 phase cells in p27^{Kip1} deficient H460 cells and excitingly they did not respond to 11-Cl-BBQ treatment (FIG. 6F). Furthermore, SA-β-gal induction after 11-Cl-BBQ treatment was also reduced in the absence of p27^{Kip1} expression (FIG. 6G). These data reveal critical roles of both p53 and p27^{Kip1} in AhR-mediated suppression of lung cancer cell proliferation by 11-Cl-BBQ.

### G. Discussion

Lung cancer is the second-most-common cancer, but it is the leading cause of cancer death-more than colon, breast, and prostate cancers combined. Much progress has been made in personalized medicine, however chemotherapy and radiation are still the primary treatment options for advanced lung cancer. Lack of effective treatment is reflected by the low 5-year survival rate of 19%, which decreases to 6% in small cell lung cancer cases. Therefore, expanding targeted anticancer drugs is critical to improve the outcomes for lung cancer patients.

Aryl hydrocarbon receptor (AhR) is a ligand-activated transcription factor and regulates cell cycle progression and cell death in a ligand-dependent manner. Significant efforts have been ongoing to exploit the anti-proliferative and pro-apoptotic functions of the AhR for cancer therapy. AbR is highly expressed in lung cancer, representing an attractive target to develop therapeutics for lung cancer treatment. Clinically approved drugs that are AhR agonists with strong anticancer effects suggest activation of AhR might be beneficial for cancer patients. 11-Cl-BBQ and its analogs are high-affinity AhR ligands that are well tolerated in mouse models. Data presented herein establishes that 11-Cl-BBQ strongly suppresses growth of lung cancer cells via an AhR-mediated induction of permanent G1 cell cycle arrest.

RNA seq analysis indicates op-regulation of CDK inhibitors such as p21^{Cip1}, p27^{Kip1} and CABLES1 (FIG. 4B, and FIG. 9A). The up-regulation of p21^{Cip1} by 11-Cl-BBQ treatment agrees with previous studies showing that p21^{Cip1} is a direct target of AhR. Data presented herein further supports that p27^{Kip1} is a direct transcriptional target of AhR and plays an important role in 11-Cl-BBQ-induced G1-phase cell cycle arrest (FIG. 6). CABLES1 is a newly identified CDK inhibitor which has been demonstrated to play important roles in cell cycle regulation and suppression of tumor growth. However, the regulation of CABLES1 mRNA expression remains poorly understood. Up-regulation of CABLES1 mRNA has been detected in transcriptomic studies using AhR ligands. Data presented herein indicates that the 11-Cl-BBQ upregulates CABLES1 mRNA as early as one hour (FIG. 4C). In addition, the expression of CABLES1 mRNA was also lower in AhR KO cells compared to AhR-expressing H460 cells (FIG. 4C). Collectively, these data suggest a role for AhR in regulating CABLES1 expression. AhR exhibits strong tumor suppressor roles in colon cancer in which knockout of CABLES1 in a colon cancer mouse model increased oncogenic Wnt/β-catenin signaling and increased cancer progression.

Gene set enrichment analysis showed that 11-Cl-BBQ suppressed genes in several pathways that are important for cell G1-S phase cell cycle progression including, G1-S phase specific transcription, DNA strand elongation, lagging strand synthesis, and E2F-mediated regulation of DNA replication (FIG. 4). Importantly, the suppression of these pathways was AbR-dependent. Treatment of Cl-BBQ (40:60 mixture of 10- and 11-Cl-BBQ) also suppressed genes enriched in cell cycle regulation pathways resulting in inhibiting the proliferation of CD4+ T cells in mice. Similarly, ligand-activated AhR has been shown to interact with the tumor suppressor retinoblastoma (RB) protein leading to suppression of E2F signaling and resulting in a G1 cell cycle arrest.

Gene ontology analysis of RNA-seq data in the current study at the transcript-level identified involvement of the tumor suppressor p53 signaling in AhR-regulated cell cycle arrest (FIG. 4B). Further investigation revealed that 11-Cl-BBQ induces p53 signaling, and that p53 was required for 11-Cl-BBQ-induced growth arrest (FIG. 5). While a slight increase in G2/M phase in p53 knockout cells treated with 11-Cl-BBQ was observed, this effect was not statically significant after applying correction for multiple comparisons (Turkey-Kramer procedure), A shift from G1 to G2 arrest in cells lacking p53 is known. A plausible explanation for this effect is that the inducing event of p53 signaling (e.g., a small molecule or ionizing radiation) may cause both G1 arrest (p53-dependent) and a G2 arrest (p53-independent). In the presence of p53, the p53-dependency dominates the effect and results in G1 arrest, whereas G2 arrest becomes more obvious in the absence of p53.

Interestingly, activation of the AhR by TCDD, the most potent and well-studied AhR ligand, does not activate p53 and has minimal effects on lung cancer cell proliferation. The mechanism by which 11-Cl-BBQ activates p53 via AhR is yet to be investigated. H460 cells express WT p53 and are highly responsive to 11-Cl-BBQ. H1299 cells have biallelic deletion of p53 gene and are relatively non-responsive. However, H69AR multidrug resistant cells harboring a p53 c.551G>T (p.E171*) mutation are still sensitive. In addition, AhR has a prominent role as a tumor suppressor in the absence of TP53 in a mouse model. Significantly increased tumor incidences and reduced survival in p53-deficient mice is associated with the loss of AhR. These findings indicate the potential of SMAhRTs that promote the tumor suppressive actions of the AhR as anti-cancer agents. We discovered CGS-15953 as one such SMAhRT that promoted AhR tumor suppressive signaling and induction of cancer cell death.

AhR mediates the growth suppression of 11-Cl-BBQ in cancer cells, such as lung cancer cells. Up-regulation of cell cycle inhibitor p27^{Kip1} and activation of p53 tumor suppressor signaling are important contributors to the potent anti-proliferative effects of 11-Cl-BBQ.

### III. BBQ Analogs as AhR Ligands with Potent Anti-Cancer Activity

### A. Introduction

Many cancers, such as triple-negative breast cancer (TNBC), have a paucity of targeted treatment opportunities. Different small-molecule AhR ligands drive strikingly different cellular and organismal responses. AhR activation by select small molecules induces cell cycle arrest or apoptosis via activating tumor suppressive transcriptional programs. AhR is highly expressed in triple-negative breast cancers, presenting a tractable therapeutic opportunity for this and other cancers. The present application identifies novel ligands of the aryl hydrocarbon receptor that potently and selectively induces cell death in triple negative breast cancer cells and TNBC stem cells via the AhR. This section concerns information specifically related to 7H-benzo[de]benzo[4,5]imidazo[2,1-a]isoquinolin-7-one (BBQ). BBQ exhibits minimal cytotoxicity against multiple normal human primary cell lines, and BBQ is well-tolerated and retains AhR-dependent bioactivity in-vivo.

AhR belongs to a superfamily of proteins involved in sensing environmental signals, with family members defined by the presence of both basic helix-loop-helix (bHLH) and PER-ARNT-SIM (PAS) domains. The receptor has a conserved role in sensing and responding to environmental and internal stimuli, binding to both endogenous and xenobiotic small-molecules and coordinating adaptive transcriptional responses to maintain organismal homeostasis. The transcriptionally inactive AhR is sequestered in the cytoplasm by multiple chaperone proteins and complex members. Upon small molecules binding to the AhR, the receptor is translocated to the nucleus where it can then interact with the heterodimeric partner aryl hydrocarbon receptor nuclear translocator (ARNT), or other transcription factors. The AhR-ARNT heterodimer complex binds to regulatory regions of DNA containing the consensus motif (5′-TNGCGTG-3′) or xenobiotic response element (XRE) and modulates gene transcription. AhR can also modulate gene expression through non-XRE elements, and interactions with other transcription factors are known, including c-MAF, KLF6, RelA and other NF-κB complex members. The transcriptional programs and resulting biological phenotypes downstream of AhR activation are highly varied, depending on tissue-specific, species-specific, and ligand-specific factors, that together influence the outcome.

While initially studied primarily in the context of xenobiotic metabolism, AhR has prominent functions in controlling the cell cycle, apoptosis and differentiation in multiple cancers. AhR activation by select ligands can induce cell cycle arrest and/or apoptosis through the induction of cell cycle regulatory proteins such as p27^{Kip1}, p21^{Cip1}, CABLES1, and cell death regulators such as p53, BMF, Fas ligand, and Bax. In certain contexts, AhR activation can enforce differentiation in both stem cells and cancer cells. Independent of exogenous ligands, AhR can exert tumor suppression, supported by in-vivo studies showing that loss of AhR drives increased tumor development and cancer hallmarks in colon, liver and prostate cancer models. In addition, AhR has been shown to restrict metastasis in lung cancer models.

In hormone-receptor positive and HER2 overexpressing breast cancers, AhR can inhibit cancer growth via multiple mechanisms, including the induction of cell cycle arrest, differentiation, and apoptosis. In triple-negative breast cancer, three AhR ligands have been identified with pro-apoptotic effects including the clinically approved drug raloxifene, a novel raloxifene analog, and CGS-15943, a pre-clinical stage molecule originally designed as an adenosine receptor antagonist. Further studies employing different ligands support AhR-dependent tumor suppressive effects, particularly inhibition of cancer cell migration.

Clinically, there is interest in antagonizing AhR activation by kynurenine, a tryptophan metabolite highly produced in tumors with high AhR expression, primarily due to its role in suppressing immune-mediated anti-tumor responses. Activating AhR signaling as a therapeutic approach, despite the potential negative impacts downstream of kynurenine signaling, can be rationalized by the understanding that: (1) different AhR ligands can drive ligand-dependent, distinct outcomes; and (ii) high-affinity, transient receptor activation results in phenotypes distinct from chronic receptor activation. Indeed, the seemingly paradoxical nature of AhR signaling, especially in cancer, has been reviewed, and is an active area of investigation.

Benzimidazoisoquinolines (BBQs) represent a novel class of high affinity AhR ligands that are rapidly metabolized and lack overt toxicity in-vivo. The AhR ligand 11-Cl-BBQ can inhibit the growth of non-small cell lung cancer cells via AhR activation and the induction of p53, p27^{Kip1}, and p21^{cip1}. The dechlorinated derivative of 11-Cl-BBQ-BBQ also is a potent AhR-dependent ligand that induces apoptosis in triple-negative breast cancer cells, with greatest potency in the Basal A subtype.

### B. Results

1. Functional Screening of Benzimidazoisoquinoline Analogs Identifies Analog 523 as an AhR Ligand with Potent Anti-Cancer Action in Triple Negative Breast Cancer Cells

In order to identify chemical analogs of 11-Cl-BBQ with AhR-dependent growth inhibitory action against triple-negative breast cancer cells, we generated cell lines lacking AhR expression using CRISPR-Cas9 gene editing. AhR knockout (KO) cell lines were generated using the CRISPR-Cas9 lentiCRISPRv2 plasmid, with single guide RNAs (gRNAs) against AhR. In parallel, matched control cell lines were generated with a lentiCRISPRv2 plasmid (CR-V2). Screening for loss of AhR expression by immunoblotting led to the identification of AhR KO clones (FIG. 10). In order to identify novel analogs of 11-Cl-BBQ, approximately 200 chemical analogs of 11-Cl-BBQ were synthesized. Analogs were screened using a three-point dose response (0.1 μM, 1 μM, 10 μM) against vector control (AbR WT) and AhR KO MDA-MB-468 cells, with relative impact on cell survival measured by Cell-Titer Glo Viability assay at 48- and 72-hours post-treatment. Of the analogs screened, 7H-benzimidazo[2,1-a]benzo[de]isoquinolin-7-one (BBQ), also referred to herein as Analog 523, shown below,

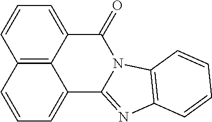 [SMILES: O=c1c2cccc3cccc(c32)c2nc3ccccc3n12]

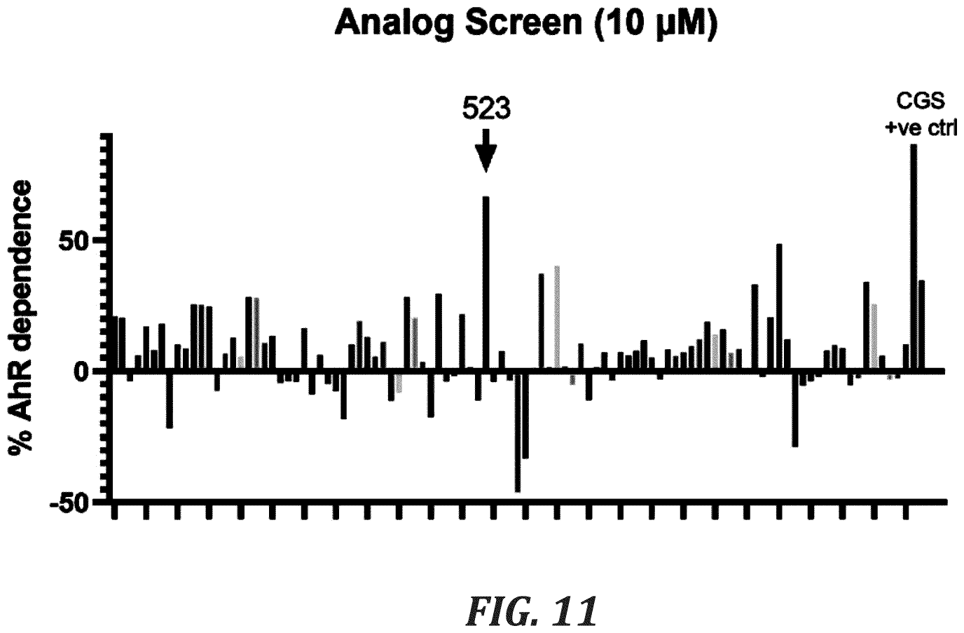

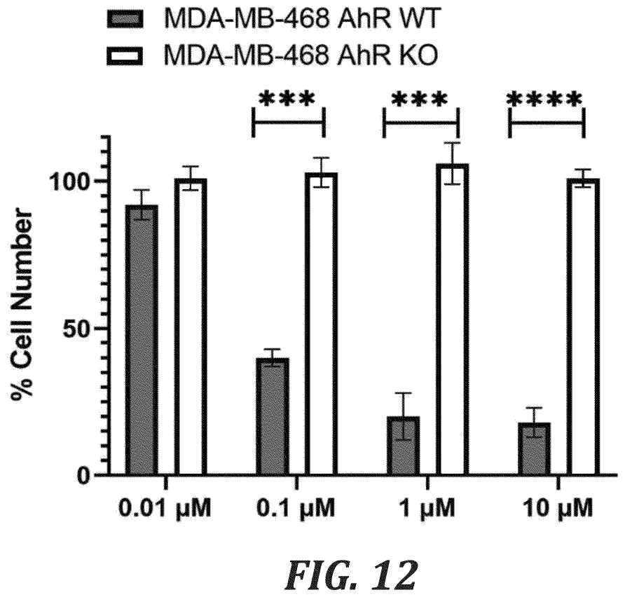

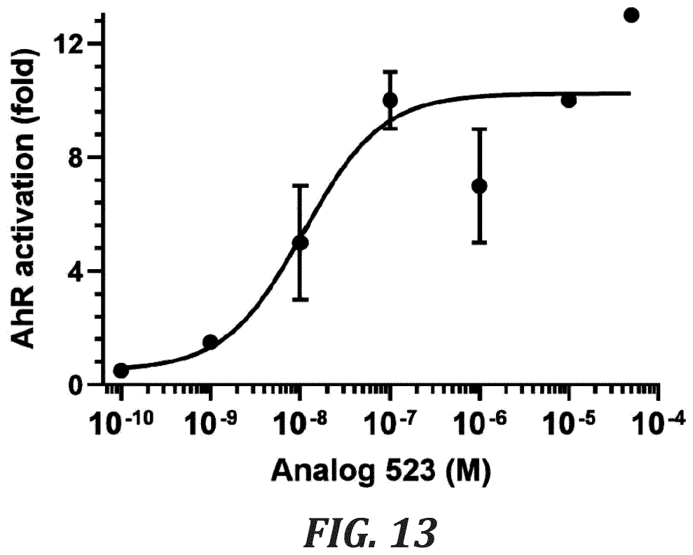

was identified as a top hit compound based on its potent (>50% inhibition at 0.1 μM), and selective activity against AhR WT cells versus AhR KO cells (>70% difference between WT and KO cells at 10 μM) (FIG. 11). While several other hits from the screen were validated, Analog 523 displayed the highest degree of potency and selectivity (FIG. 12), and thus was chosen to move forward with further studies. Surprisingly, Analog 523 is structurally very similar to 10-Cl-BBQ and 11-Cl-BBQ, and differs only by the loss of the chlorine atom on the benzimidazole ring, resulting in Mouse Hepa-1 cells expressing a xenobiotic response element driven reporter were treated with Analog 523 for 16 hours (FIG. 13) to determine whether Analog 523 could activate AhR-dependent transcription. Receptor activation was observed at concentrations as low as 100 μM (˜2 fold) and maximal reporter induction was achieved at approximately 100 nM (˜10-12 fold). This confirmed Analog 523 activates AhR-mediated transcription and functions as an AhR ligand.

### 2. Analog 523 Possesses AhR-Dependent Anti-Proliferative and Pro-Apoptotic Activity in Triple-Negative Breast Cancer Cells

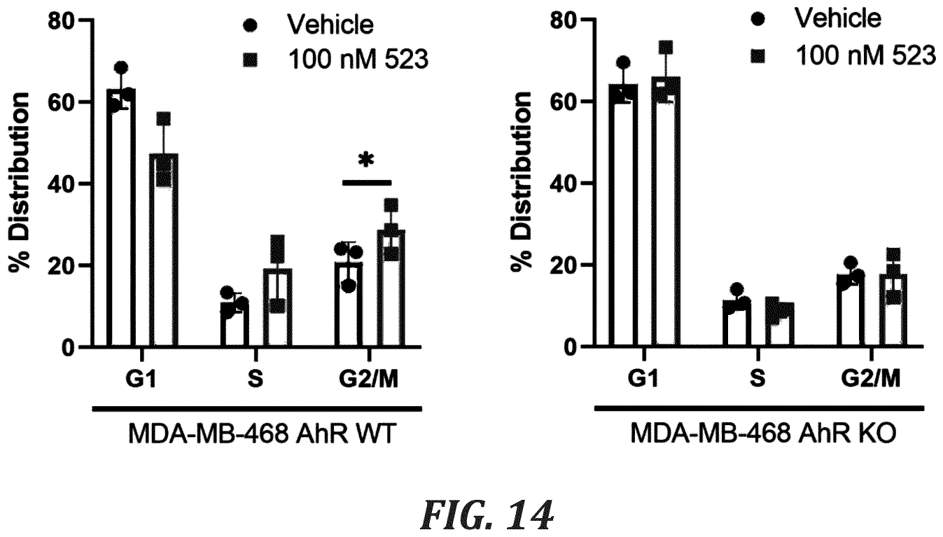

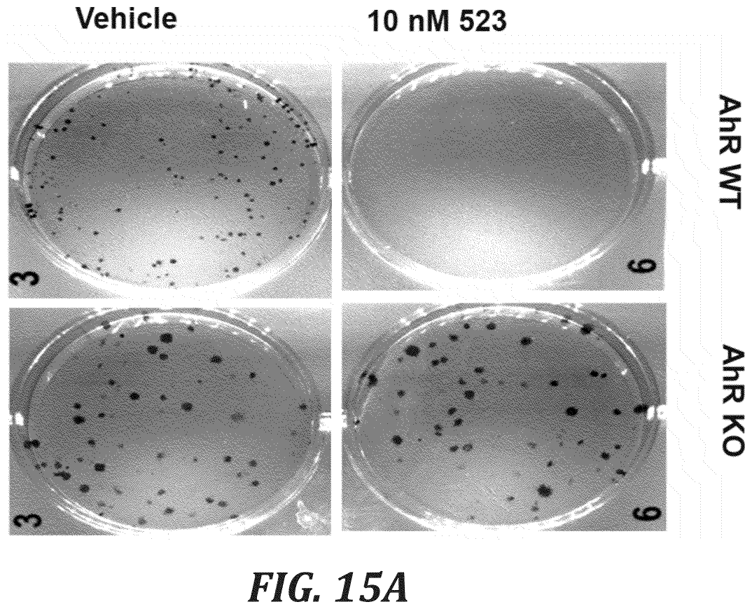

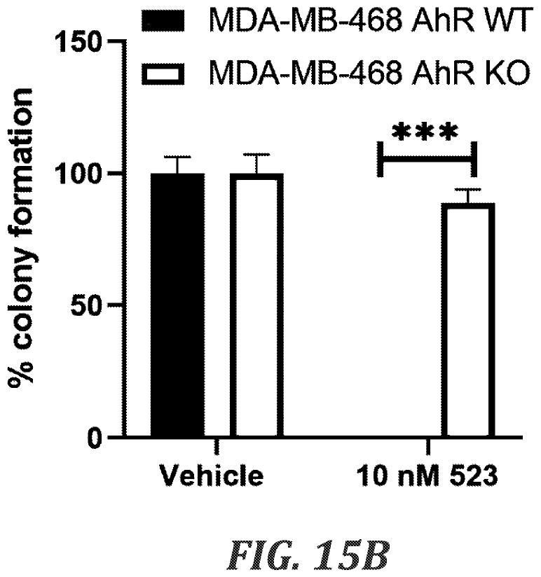

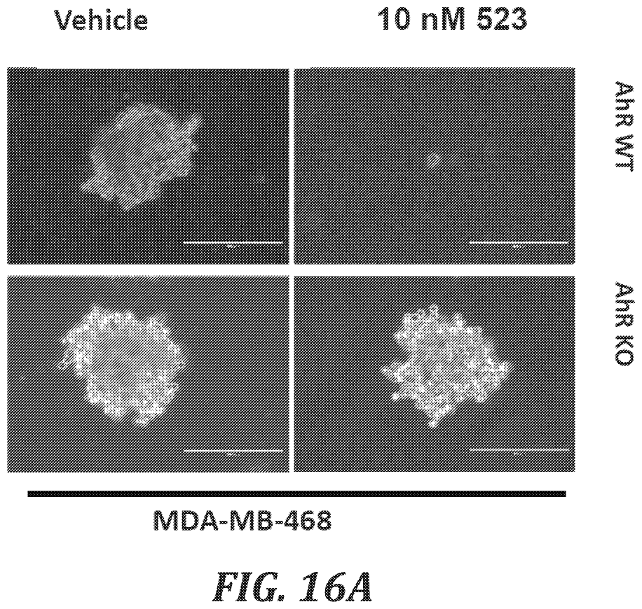

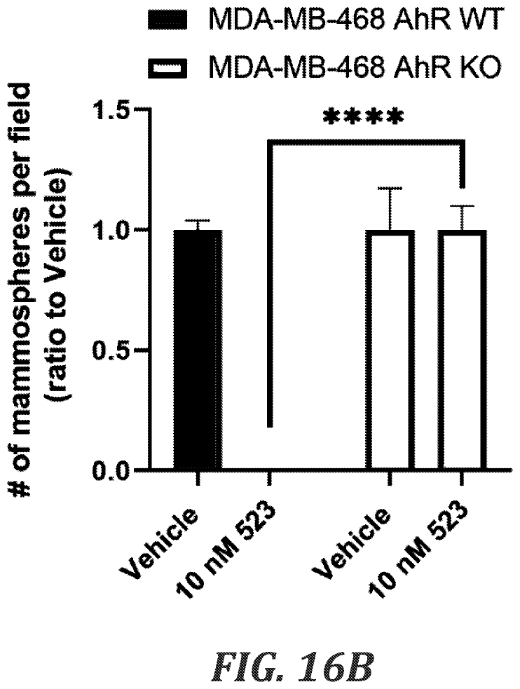

Given the potent AhR-dependent activity of Analog 523, the anti-cancer effects in the MDA-MB-468 cell line were further characterized. Short-term (24 hour) treatment with 100 nM 523 induced an AbR-dependent S/G2 phase arrest (FIG. 14), and long-term exposure to 10 nM 523 in a colony forming assay was sufficient to completely inhibit the clonogenicity of AhR-expressing cells while exhibiting minimal impact on AhR-deficient cells (FIG. 15). Similarly, in the soft-agar clonogenicity assay, an in-vitro measure of metastatic potential, Analog 523 potently suppressed 3D colony formation at 10 nM (FIG. 16A, B) in an AhR-dependent manner.

3. Analog 523 Induces AhR-Dependent Apoptosis in Cancer Cells, but does Not Inhibit Normal, Primary Cell Growth

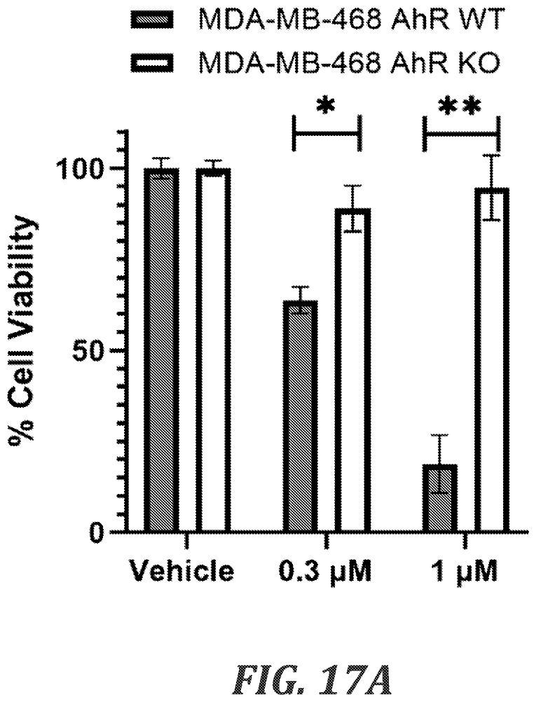

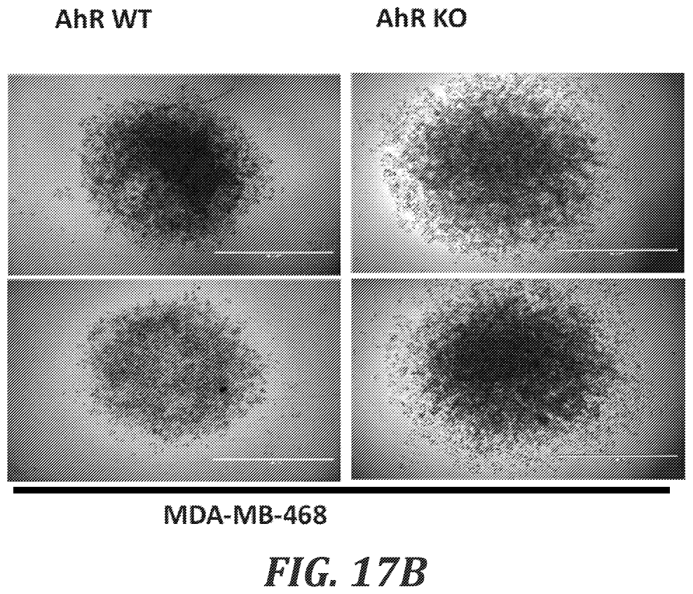

To determine the effectiveness of Analog 523 in 3-dimensional cultures, a more physiologically relevant model of solid tumors, spheroid cultures of MDA-MB-468 cells were formed. We exposed spheroids to Analog 523 for 72 hours and observed strong, AhR-dependent growth inhibition as measured by viability assay. Comparison of the dose-response curves from 2-dimensional (FIG. 12) and spheroid viability assays (FIG. 17) revealed some loss of efficacy in spheroid culture at 0.3 μM, although this difference was minimal at 1 μM.

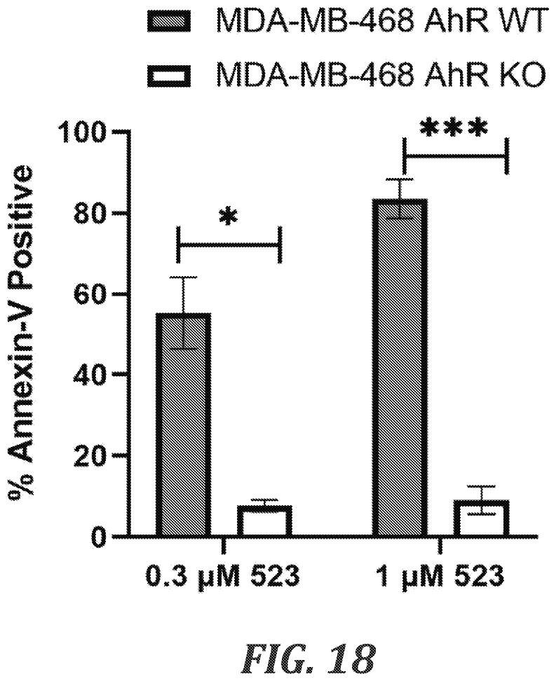

It was then determined whether Analog 523 could induce apoptosis. Annexin-V staining of AhR WT and AhR KO cells exposed to Analog 523 revealed potent AhR-dependent induction of apoptosis (FIG. 18). AhR is expressed in TNBC tumor tissue but is also significantly expressed in normal mammary epithelial cells (Cite human tissue expression). To determine whether AhR activation in normal mammary epithelial cells would induce any undesired cytotoxicity, both primary human mammary epithelial cells (HMECs) and the non-malignant mammary epithelial line MCF10A were used to evaluate if Analog 523 inhibited cell viability. Treatment with concentrations between 0.01-10 μM did not induce any cytotoxicity, while treatment with 10 μM 523 minorly inhibited cell viability (˜20%). Similarly, the effect of Analog 523 on HEK293T and normal primary human fibroblast viability was tested, representing a non-tumorigenic transformed cell line and non-cancerous cells from a tissue distinct from mammary epithelial cells. Together this data supported Analog 523 acting as a cancer selective AbR agonist, with minimal effects on normal breast epithelial cells.

### 4. Analog 523 Inhibits the Growth of Multiple TNBC Cell Lines

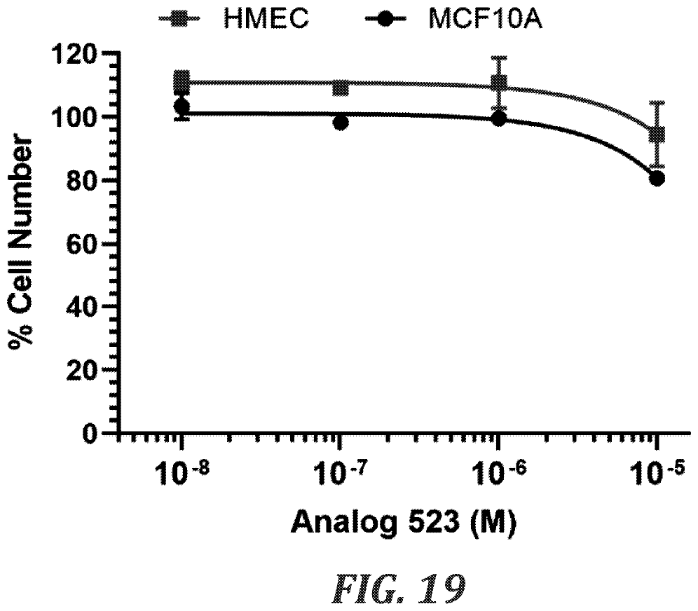

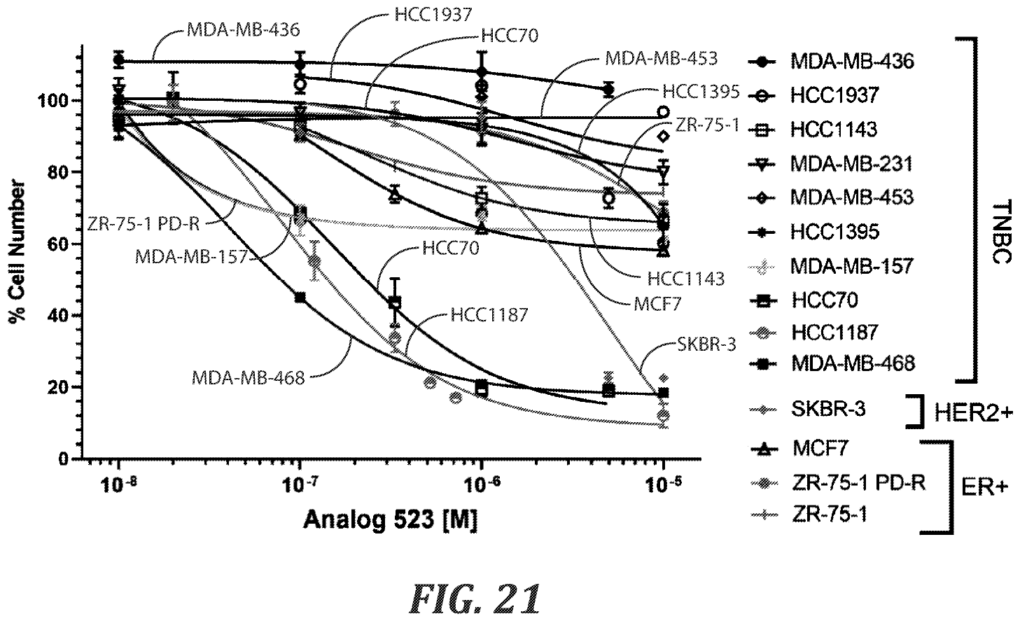

The ability of Analog 523 to inhibit a panel of triple-negative breast cancer cell lines, in addition to ER-positive MCF-7, cells ZR-75I cells, and ZR-75I cells resistant to the CDK4/6 inhibitor PD-0332991 was profiled using a cell viability assay (FIG. 21). Together with the MDA-MB-468 cell line (Basal A subtype), HCC1187 cells (Basal A; Immunomodulatory) and HCC70 cells (Basal A), were highly sensitive (>50% inhibition at <0.5 μM) to 523 treatment. In addition, the HER2+SKBR3 cell line was also sensitive to 523 at concentrations >1 μM. The remaining cell lines profiled exhibited moderate or minimal sensitivity to Analog 523, with inhibition <50% at the concentrations tested. Treatment of HCC70 and HCC1187 cells with 1 μM of 523 for 48 hours strongly induced apoptosis (FIG. 18 and FIG. 19), consistent with the sensitivity observed by viability assay.

### 5. Analog 523 Suppresses TNBC Stem-Cells Via AhR

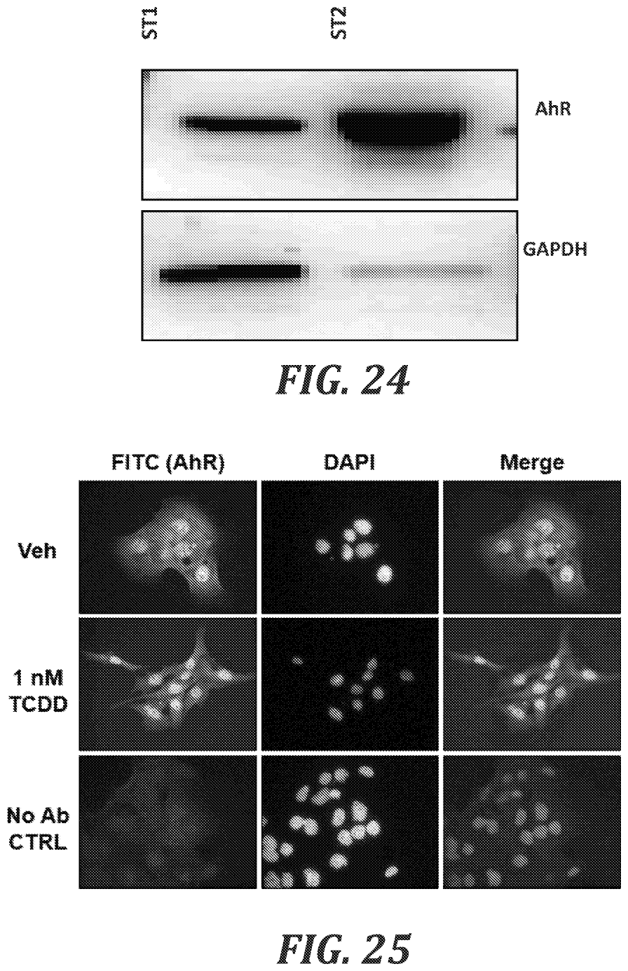

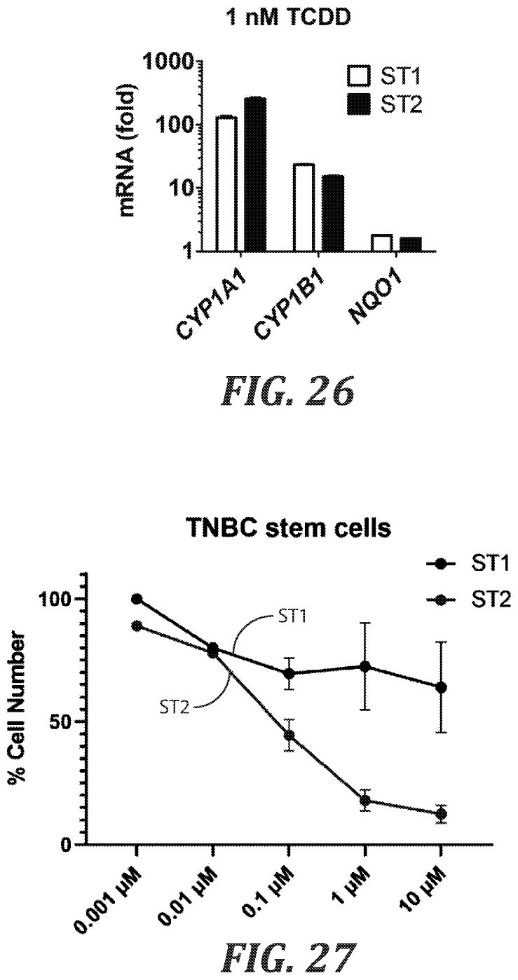

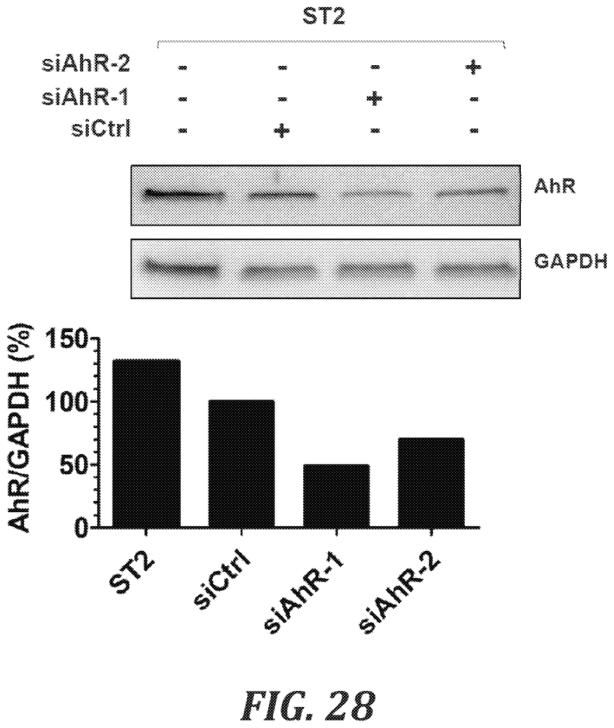

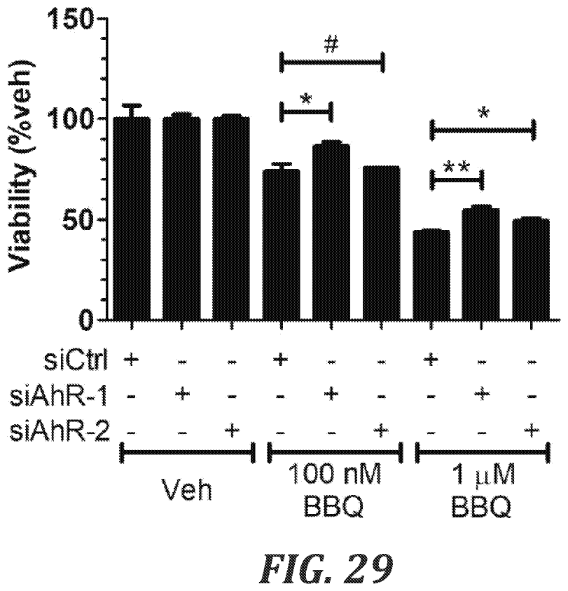

Cancer stem cells represent sources of proliferation, drug resistance and metastasis in triple-negative breast cancers. The effects of 523 on cancer stem cells (CSCs) isolated from triple-negative breast cancer xenografts was therefore determined. Two clonal populations were isolated, designated ST1 and ST2. The levels of AhR expression in these populations was first determined, and found that both expressed high levels of AhR, with the ST2 clone expressing much greater levels than the ST1 clone (FIG. 24). Whether the AhR pathway was functional in these cells was next determined. Treatment with the AhR ligand TCDD induced nuclear translocation of AhR, as determined by immunocytochemical staining of AhR in ST2 cells treated with vehicle or 1 nM TCDD (FIG. 25). AhR was confirmed to be transcriptionally active in these cells by measurement of AhR target genes CYP1A1, CYP1B1, and NQO1, following treatment with 1 nM TCDD (FIG. 26). Whether Analog 523 inhibited the growth of these cells was next determined by cell viability assay and found that while both the ST1 and ST2 cells were sensitive to 523, ST2 cells were far more sensitive, with 100 nM of Analog 523 producing >50% inhibition of viability (FIG. 27). The greater sensitivity of the ST2 cells to Analog 523 is consistent with these cells expressing much higher levels of AhR relative to the ST1 counterparts. Whether the inhibitory effects observed were dependent on AhR was determined. Small-interfering RNA (siRNA) was used to knockdown AhR expression (FIG. 28). ST2 cells transfected with scramble controls were more sensitive to treatment with 0.1 μM or 1 μM 523 relative to those with AbR expression knocked down (siAhR) (FIG. 29).

### 6. Transcriptional Profiling of Analog 523 in TNBC Cells

To determine the transcriptional programs downstream of AhR following activation by Analog 523, MDA-MB-468 AbR WT and MDA-MB-468 AhR KO cells were with 250 nM 523, and collected samples at 4 hours and 12 hours post-treatment for global gene expression profiling. Four hours was selected as a timepoint that would capture a significant fraction of the early, direct AhR-dependent transcriptional targets of Analog 523. Most of the fraction of cytosolic AhR has translocated to the nucleus following 2-3 hours of ligand treatment with Analog 523 (FIG. 25). Twelve-hours post-treatment was selected as a secondary timepoint to capture post primary and consequent secondary responses. AbR WT cells treated with Analog 523 for 4 hours and 12 hours shared a core set of 107 differentially expressed, upregulated genes at both 4 and 12 hours (Overlap p-value=1×10⁻¹⁵⁶) (adjusted p-value <0.05, log 2 (FC)>1). At 4 hours and 12 hours post-treatment, 148 genes, and 157 genes were up-regulated respectively.

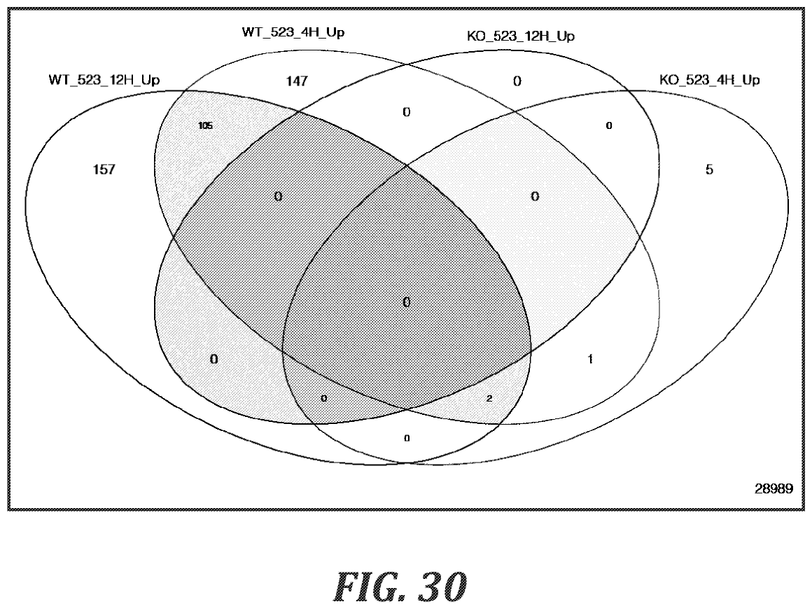

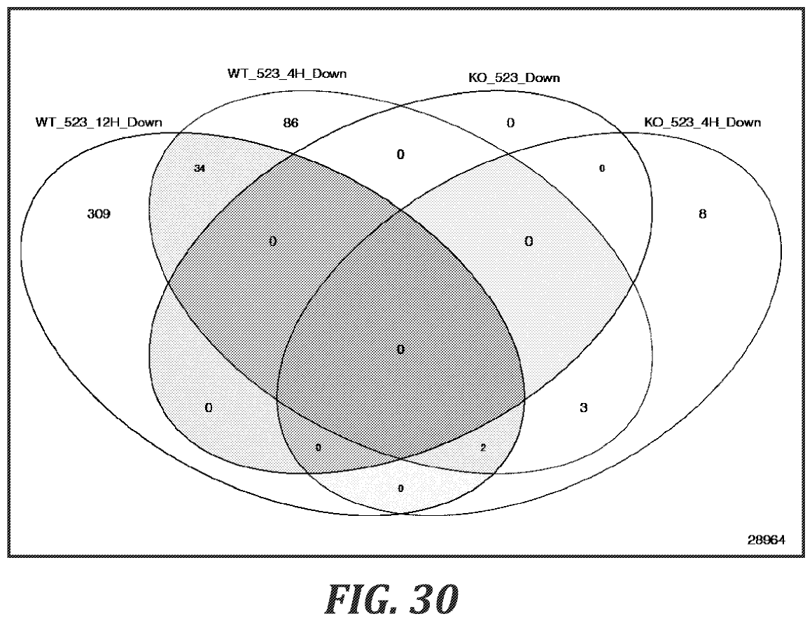

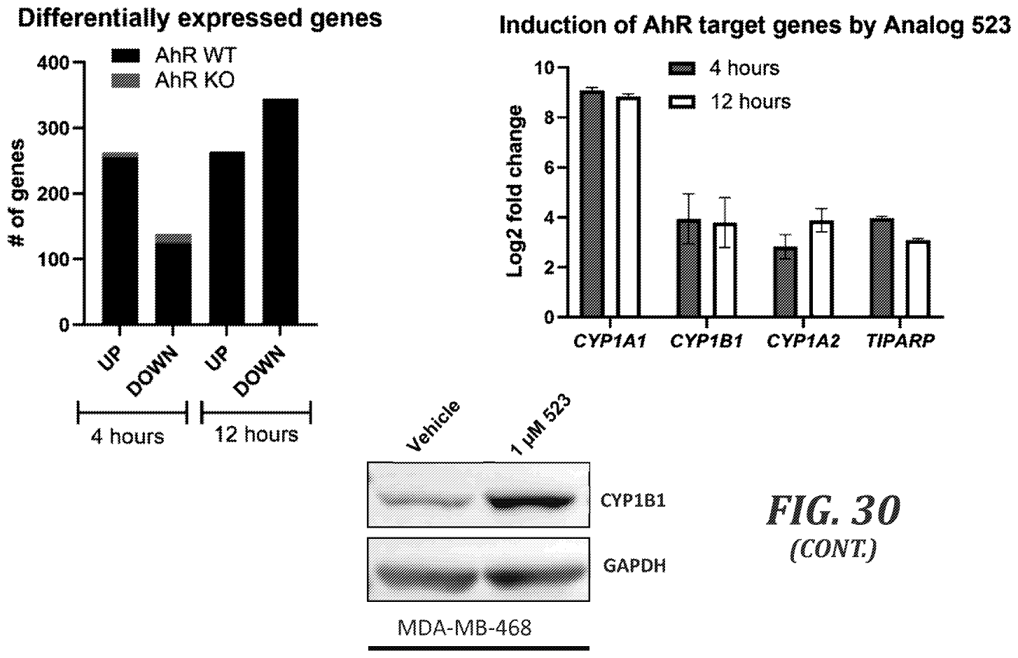

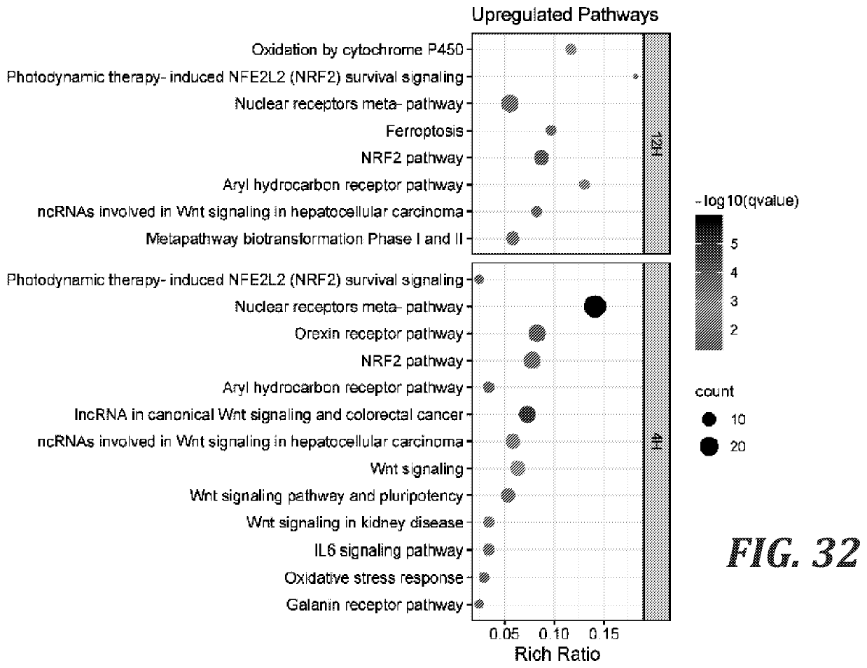

Gene Ontology (GO TERM) analysis and gene set enrichment analysis was used to determine the AhR-dependent biological processes and pathways downstream of Analog 523. Consistent with Analog 523 activating AbR-regulated transcription programs, induction of well-established AhR transcriptional targets was observed including TIPARP, CYP1A1, CYP1A2, and CYP1B1 (FIG. 30). Functional enrichment analysis using the g: Profiler analysis platform, and gene set enrichment analysis by annotation with The Molecular Signatures Database (MSigDB v7.5.1) (Liberzon et al., 2015) was used to determine pathways (WikiPathways) activated or repressed downstream of Analog 523 treatment (FIG. 32).

Profiling of pathways enriched 4 hours after Analog 523 treatment identified networks related to nuclear receptor signaling, orexins, Wnt signaling, oxidative stress regulator NRF2, and AbR signaling. Enrichment of these pathways is consistent with previously reported functions of AhR signaling. For instance, disruption of Wnt signaling by AhR expression or activation has been previously reported. AhR-dependent induction of oxidative stress and NRF2 activation is a well-established signaling network. The transcription of genes involved in orexin signaling following AhR activation has also been reported. The nuclear receptors meta-pathway represented the most highly enriched annotation in terms of gene number, overlap and significance, although is likely only broadly indicative of AhR pathway activation. Alteration of NRF2-regulated targets, and Wnt signaling also was significantly represented at both 4 and 12 hours in terms of DEG number and pathway overlap. At 12 hours post-treatment with Analog 523, enriched pathways included NRF2 signaling, Wnt signaling, benzo[a]pyrene metabolism, and oxidation by cytochromes p450, consistent with pathways upregulated at 4 hours post-treatment, and aligning with known functions downstream of AhR activation such as induction of xenobiotic metabolizing enzymes. In addition, induction of gene networks regulated by p53, and pathways involved in ferroptotic cell death were observed.

GSEA with The Molecular Signatures Database (v7.4 MSigDB) was used to identify transcriptional signatures upregulated at 4- and 12 hours post-treatment with Analog 523. Hallmark Gene Sets derived from upregulated targets represented a wide range of processes. Prominently enriched at both 4 and 12 hours was TNF-α signaling via NF-κB (39/200, FDR q-value=4.15×10⁻³², p-value=2.29×10⁻³⁶). Among the enriched transcriptional signatures were apoptosis at 4 hours post-treatment and the p53 signaling pathway at both 4 and 12 hours.

Inspection of these targets identified pro-apoptotic mediators including BH3-only proteins such as BMF (4-5 fold), BIK (˜1.5 fold), and PMA1P1 (˜1.5 fold). In addition, transcriptional mediators related to extrinsic apoptosis signaling such as TNFRSF12A, IRF, and IL6 were activated. Also induced were genes such as stress-inducible GADD45α, p53 target-gene HMOX1 (Meiller et al., 2007), and SMAD7 which has anti-proliferative effects in breast cancer.

Induction of p53 transcriptional targets at 4 and 12 bours revealed a common set of genes activated at both timepoints: IER3, PLK3, OSGIN1, LIF, FUCA1, SLC3A2, and HBEGF. Immediate early response gene 3 (IER3) and oxidative stress induced growth inhibitor 1 (OSGIN1) exhibited the highest inductions among these targets with greater than 6-fold and 4-fold increases at 12 hours respectively. Further data analysis of curated gene sets revealed mammary-specific p53 transcriptional signature (68/1197, FDR q-value=5.44×10⁻²⁵, p-value=5.13×10⁻²⁸), and a curated signature overlapping with doxorubicin-induced apoptosis in breast cancer cells (65/1153, FDR q-value 8.47×10⁻²⁴, p-value=1.33×10⁻²⁶), at 4 hours post-treatment.

The AhR-dependent induction of p53 signaling and the oxidative stress response may contribute to the apoptotic phenotype observed in response to Analog 523 treatment. MDA-MB-468 cells express only mutant p53 with an R²⁷³H substitution. R²⁷³H p53 mutant cells display increased sensitivity to ferroptosis relative to p53 wild-type cells.

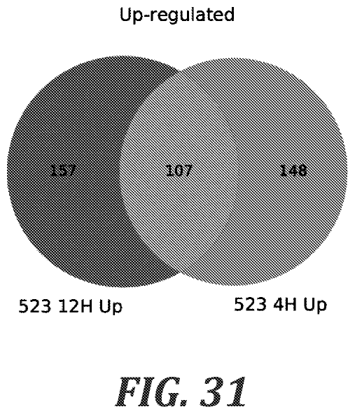

There were 86 and 309 genes downregulated by greater than 2-fold (p<0.05) at 4 hours and 12 hours respectively, with a core set of 36 genes shared between the two timepoints (Overlap p-value=1×10⁻³⁹) (FIG. 31). AhR KO cells showed minimal or no transcriptional changes within the fold-change and significance parameters applied, with 13 upregulated and 8 downregulated genes at 4 hours, and 0 genes differentially expressed at 12 hours respectively, indicating highly AhR-dependent activity (FIG. 30).

Gene networks downregulated 4 hours post-treatment with Analog 523 included a diverse range of biological pathways involved in processes such as oligodendrocyte myelination, ossification and glucocorticoid receptor signaling. AhR-dependent control of genes related to these processes has been previously reported.

### 7. Discussion

The aryl hydrocarbon receptor (AhR) can inhibit both the development of cancer, and can be therapeutically targeted by select ligands which elicit tumor suppressive gene programs. Tumor suppressive signaling downstream of AhR has been implicated in breast cancer and multiple other cancer types, including lung cancer, colon cancer, glioblastoma, medulloblastoma, hepatocellular carcinoma, melanoma and leukemia.

AbR expression, and activation by exogenous ligands can suppress cancer growth through a variety of mechanisms, including the induction of cell cycle and cell death regulators, and the restriction of stemness programs. Benzimidazoisoquinolines (BBQs) have been identified as high affinity AhR ligands that are rapidly metabolized and do not exert overt cytotoxicity in vivo. In this context, BBQs as a class were shown to induce regulatory T-cells via AhR activation and prevent murine graft-versus-host disease. Analog 523 (BBQ) in this context was found to be well-tolerated and retained bioactivity in vivo. Recently, the structural isomer of 10-Cl-BBQ—11-Cl-BBQ—was shown to possess potent AhR-dependent effects against lung cancer cells via AhR-dependent activation of p53 signaling and induction of p27^{Kip1} and p21^{Cip1}. 11-Cl-BBQ was found to induce an irreversible cell-cycle arrest in lung cancer cells but did not induce apoptosis. Analog 523 (BBQ) is identified herein as an example of a potent, cancer selective, inducer of apoptosis in triple negative breast cancer cells that acts via AhR.

To identify novel structural analogs of 11-Cl-BBQ with increased anti-cancer activity, more than 400 BBQ analogs were profiled in MDA-MB-468 AhR WT and KO cells to select for compounds that exhibited AhR-dependent inhibition of cancer cell viability. Interestingly, Analog 523, was found to have the greatest degree of AhR-selectivity and potency across the concentrations screened (FIG. 11). Using the MDA-MB-468 cells as a model system, we determined that low concentrations (10-100 nM) of Analog 523 induces an AbR-dependent G2/M phase cell cycle arrest and is sufficient to completely inhibit colony growth (FIGS. 14-16). Exposure to higher concentrations of Analog 523 (0.3-1 μM) for longer periods (48-72 hours) drives AhR-dependent apoptosis (FIG. 15A, B). In two primary breast epithelial cell lines, and normal, primary human fibroblasts, Analog 523 did not exert significant cytotoxicity (FIGS. 16A and 16B).

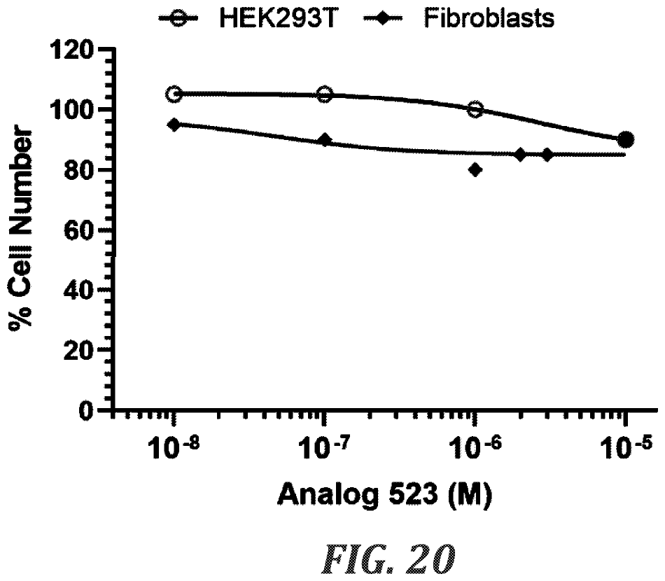

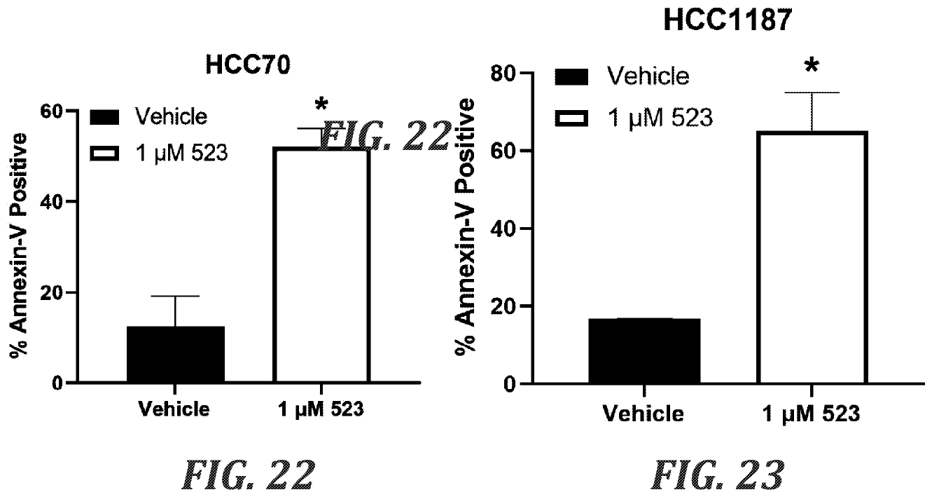

The AhR-dependent effects of Analog 523 in a panel of triple-negative breast cancer cell lines have been determined, in addition to ER-positive and HER2-amplified breast cancer cell lines. Of the additional TNBC cell lines surveyed, the HCC1187 and HCC70 cell lines emerged as highly sensitive to Analog 523 (HCC1187 IC50: 81.7 nM; HCC70 IC50: 153.4 nM), while MDA-MB-468 cells remained the most sensitive breast cancer cell line identified (IC50: 41.2 nM) (FIGS. 17-20). In TNBC stem cells, Analog 523 strongly inhibited cell proliferation, and this effect was driven by AhR (FIGS. 21-23). AhR was found to be highly expressed, and functional in these cells, and indicating a tractable opportunity to target this aggressive subpopulation.

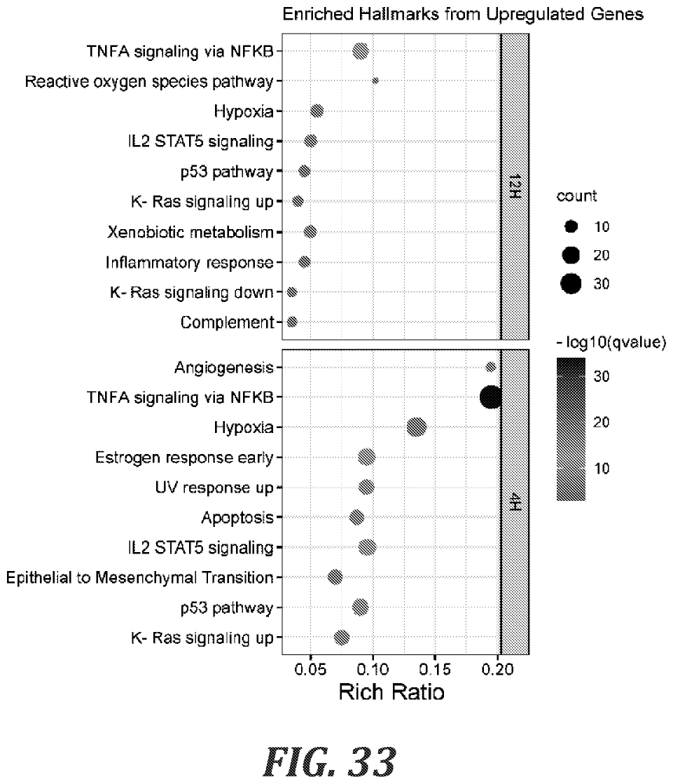

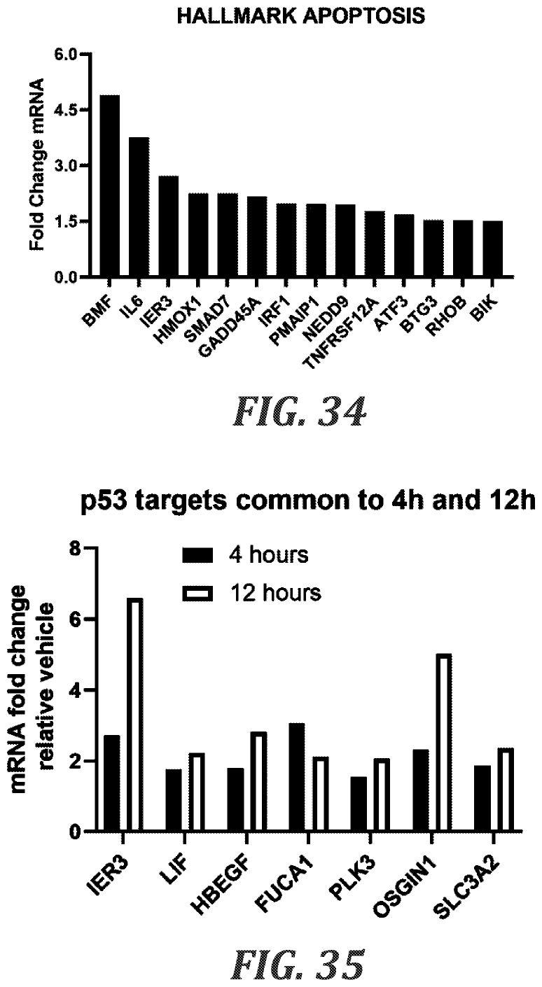

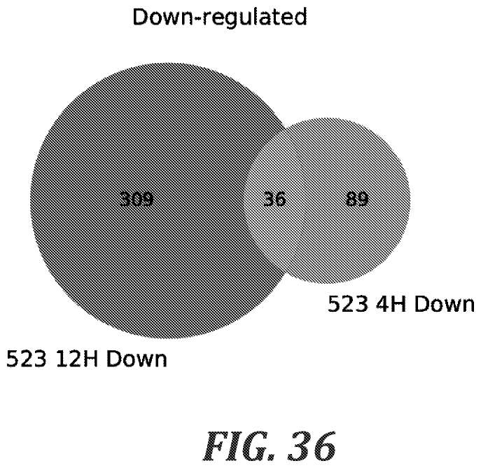

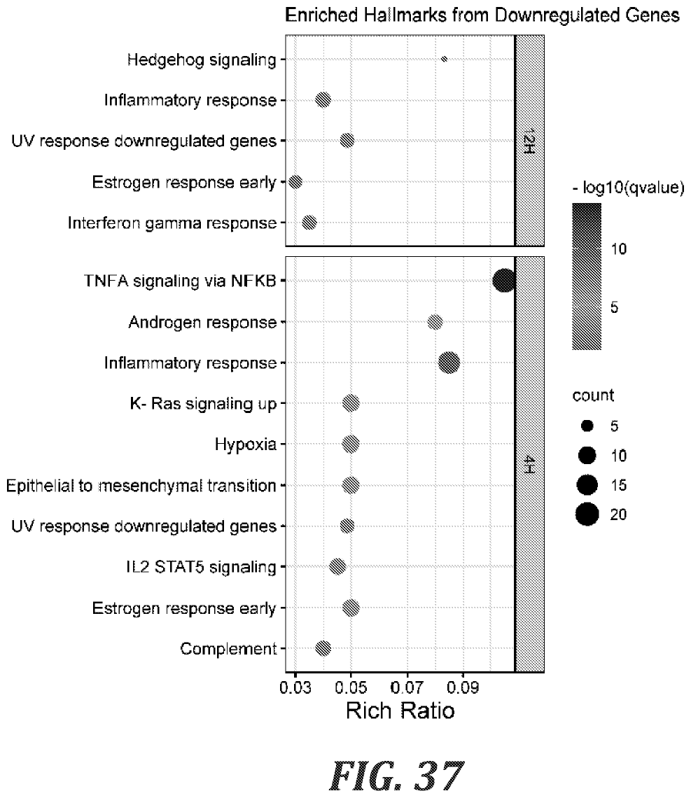

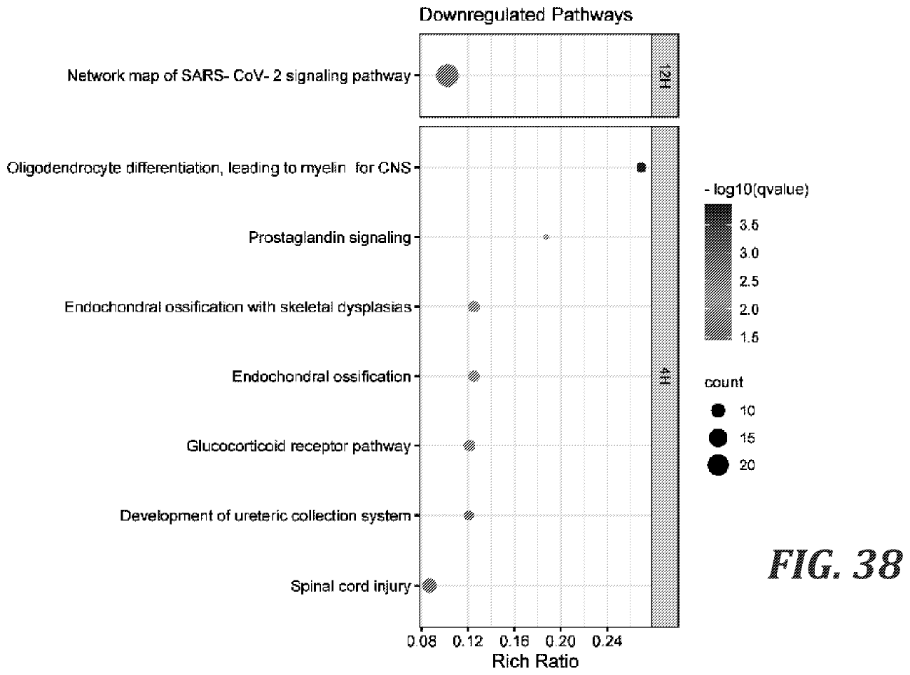

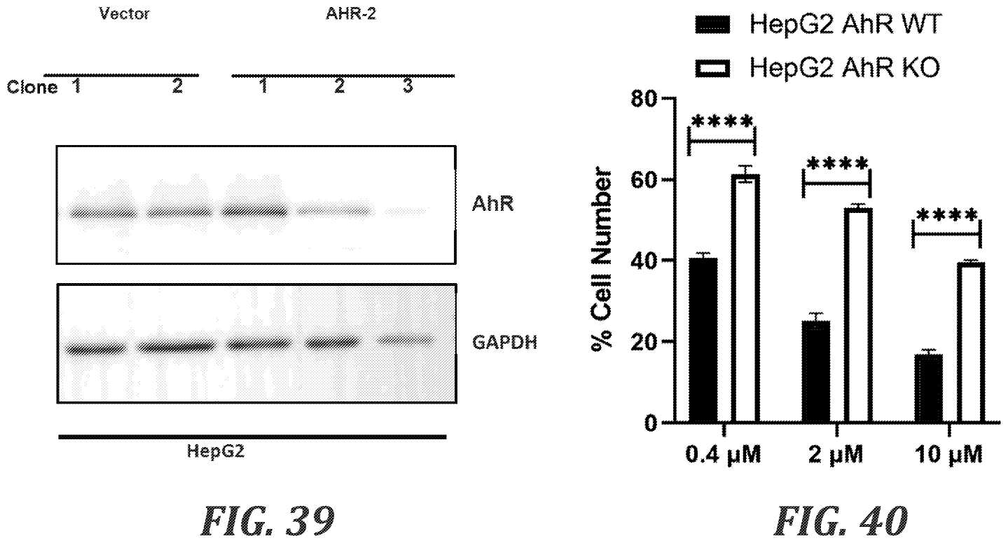

To determine the transcriptional programs downstream of AhR in TNBC cells, global gene expression profiling by RNA-Seq was used. At both 4 hours and 12 hours post-treatment, induction of gene targets downstream of p53 was observed, and involved in apoptosis (FIG. 33). Furthermore, enrichment of signatures corresponding to NRF2 transcriptional targets was observed, Wnt signaling, ferroptosis, and TNF-α signaling via NF-κB (FIGS. 32, 33). Analog 523 induced transcription of BH3-only pro-apoptotic Bcl-2 family members including BMF, BIK, and PMAIP1, and activated transcription of different stress-response factors (FIGS. 34, 35). OSGIN1 represents a gene transcriptionally regulated by both NRF2 and p53 (FIG. 35) and is a stress-inducible target gene, that can functionally interact with p53. PLK3 and GADD45a are two stress-inducible targets involved in the G2/M checkpoint that may be involved in promoting the anti-proliferative and pro-apoptotic effects observed in response to Analog 523 (FIG. 15A, B). PLK3 can phosphorylate and activate p53 in response to various stress signals such as reactive oxygen species and hypoxia. Loss of AhR in p53-deficient, or p53-heterozygous backgrounds resulted in increased tumor incidence and tumor spectrum when compared with AhR wild-type counterparts, suggesting that AhR interacts with the p53 signaling pathway, and may partially compensate for the oncogenic effects of p53 loss. Previous studies indicate that AhR activation by non-genotoxic ligands can increase p53 expression. Furthermore, the related transcription factor, and heterodimer partner of AhR-ARNT can modulate p53 signaling.

BBQ compounds disclosed herein, including Analog 523, are well-tolerated and are biologically active in-vivo at clinically achievable concentrations (10 mg/kg). Previous toxicity studies determined a relatively high LD₅₀ value of 660 mg/kg in rats (https://chem.nlm.nih.gov/chemidplus/rn/23749-58-8), supporting a large therapeutic window. 10-Cl-BBQ and an analog NAP-6 were shown to possess anti-cancer activity in triple-negative breast cancer cells, and some of the important structural features governing activity were determined. The naphthylene ring and ortho-disposed substituents on the N-phenyl ring may be important for AhR functional activity.

Transient activation of AhR signaling by many ligands is not inherently toxic, and moreover, AhR represents an attractive drug target for many diseases. AhR agonists are currently being tested in clinical trials in the context of autoimmune diseases such as multiple sclerosis. The recent approval of the AbR ligand Tapinarof for the topical treatment of psoriasis supports the feasibility of translating AhR active compounds for cancer therapy. Furthermore, many approved drugs are in fact AhR agonists, and hold repurposing potential as anti-cancer agents.

Although an in-depth mechanistic understanding of the essential molecular targets downstream of AhR activation by Analog 523 remains to be determined, Analog 523 acts as a potent AhR agonist with cancer cell selective anti-proliferative and pro-apoptotic activity. Analog 523 activates transcription of both intrinsic and extrinsic cell death mediators, and induces p53 signaling in triple negative breast cancer cells and TNBC stem cells. We propose Analog 523 as a candidate for clinical translation as an anti-cancer agent.

i. AhR-Dependent Pro-Apoptotic Effect of 523 in Hepatocellular Carcinoma

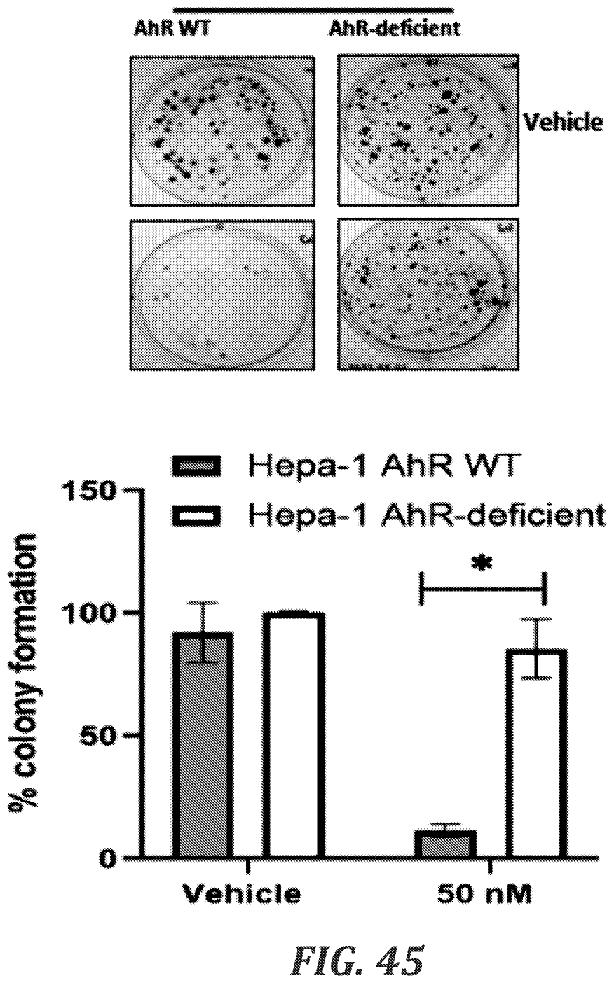

Select AbR ligands exert anti-cancer activity in hepatocellular carcinoma. To determine the AhR-dependent effects of Analog 523 in these cells, clones deficient in AhR expression were generated using CRISPR-Cas9 (FIG. 31). A clone transfected with the AhR-2 sgRNA that exhibited significantly reduced AhR expression was isolated and used characterize the AhR-dependent effects of 523. Viability assay showed significant dose- and AhR-dependence in response to Analog 523 for 72 hours (FIG. 32). Annexin-V staining for apoptotic cells following 48-hour treatment with 2 μM 523 revealed strong AhR-dependent induction of cell death in vector controls, while AhR deficient cells were minimally affected (FIG. 33). The effects of Analog 523 in the mouse hepatoma cell line Hepa-1 and its derivative cell line Hepa-1 TAO were characterized and exhibited 90% reduced AhR expression (FIG. 34). 48-hour exposure to Analog 523 potently inhibited cell viability of Hepa-1 cells at sub-micromolar concentrations, while minimally affecting Hepa-1 TAO cells deficient in AhR expression (FIG. 35). Treatment with 100 nM and 500 nM of Analog 523 for 72 hours also induced a robust apoptotic response (FIG. 44). Exposure to low concentrations of Analog 523 (10-50 nM) were sufficient to strongly inhibit colony formation in an AhR-dependent manner (FIG. 45).

To gain insight into the apoptotic pathways activated by Analog 523 in Hepa-1 cells, a focused RT-qPCR gene-array was used. Hepa-1 cells were treated with 300 nM of Analog 523 for 24 hours before harvesting RNA. Profiling revealed enrichment of extrinsic cell death mediators including TNF, FasL, TRAIL (TNFSF10), Fas, TRAP (CD40L), TWEAK (TNFSF12), TNFRSF1A, CD70 and CASP8. In addition, among the most highly upregulated genes were tumor suppressors p63 and p73, and MAPK1. FasL was induced by the AhR ligand CGS-15943 in Hepa-1 cells and was required for AhR-induced apoptosis, suggesting these two ligands may induce cell death through similar downstream mechanisms.

CGS-15943, shown below, is a molecule originally developed as an adenosine receptor antagonist, that acts as an AhR ligand with strong anti-cancer activity in hepatoma cells and triple-negative breast cancer cells.

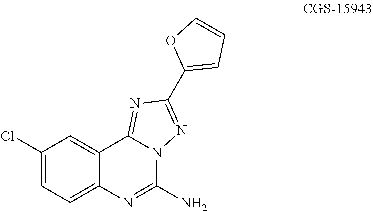 [SMILES: Nc1nc2ccc(Cl)cc2c2nc(-c3ccco3)nn12]

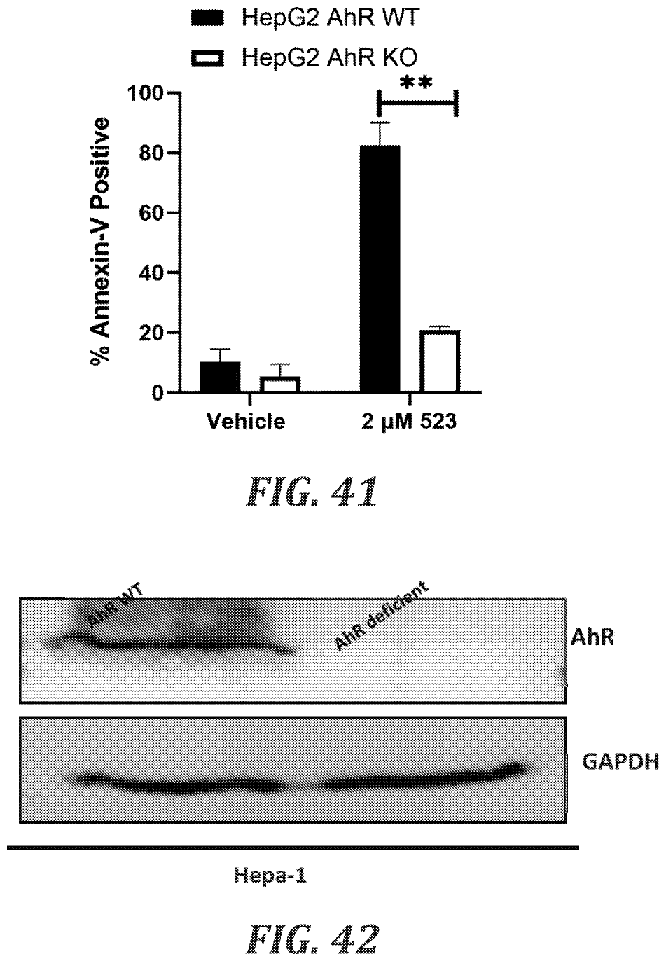

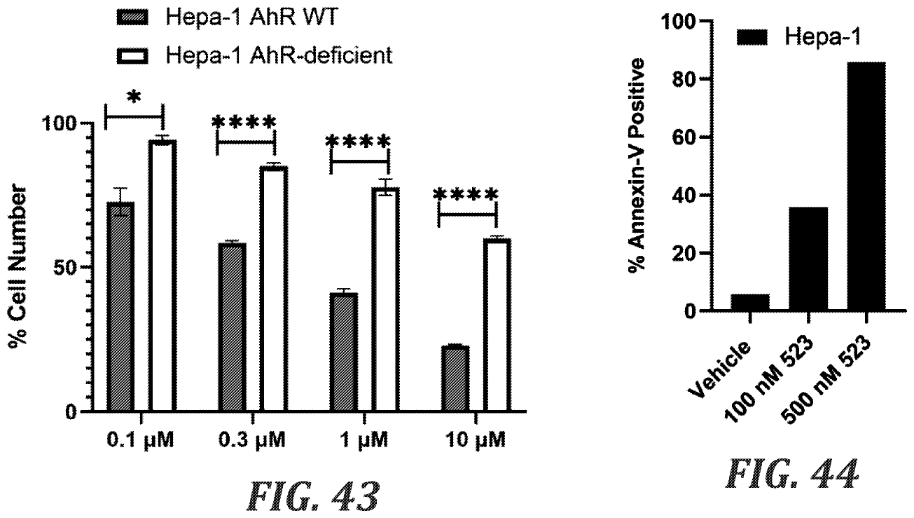

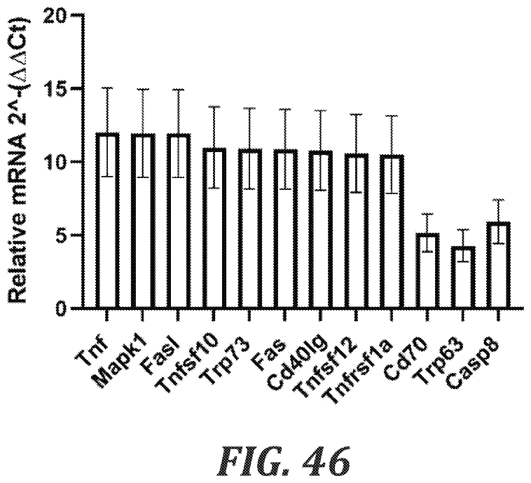

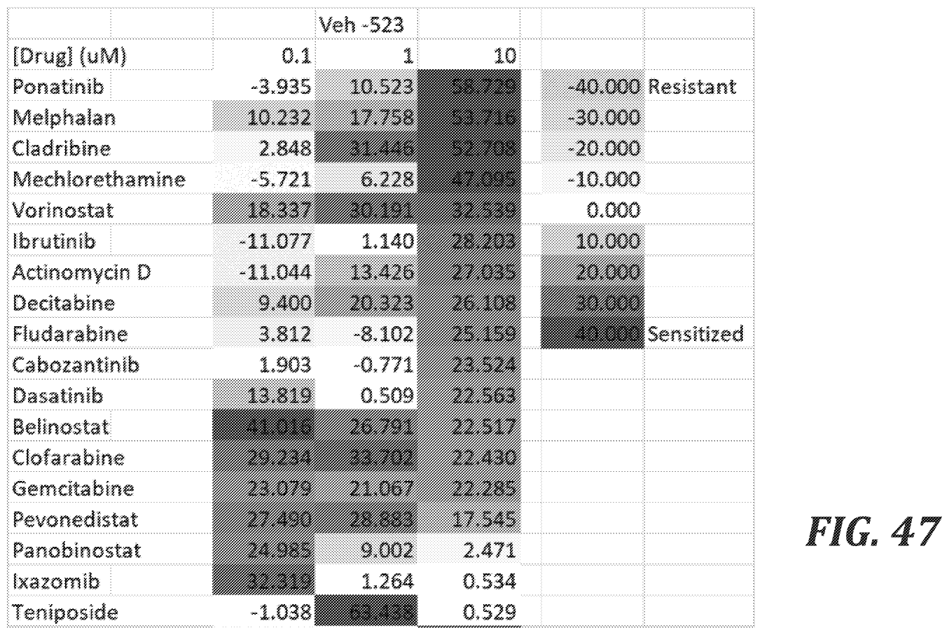

Like Analog 523, CGS-15943 induces AhR-dependent apoptosis in hepatoma cells (FIGS. 41, 44), although the two ligands share only minor structural overlap. CGS-15943 was found to drive Fas ligand (FasL) expression and induction of the Type II extrinsic cell death pathway in Hepa-1 cells. Similarly, in Hepa-1 cells, Analog 523 drives induction of FasL, in addition to other extrinsic cell death mediators (FIG. 46), suggesting these two ligands may share common mediators downstream of AhR.

AhR activation sensitizes immune cells to Fas-mediated apoptosis, and can also sensitize hepatocytes to Fas-mediated apoptosis. Further work has shown AhR as an effector of pro-apoptotic signaling downstream of TNF-α. Additionally, AhR and NF-κB can cooperate to regulated Fas and FasL transcription. The activation of death receptor (TNF-α/FasL) and NF-κB-regulated targets downstream of AhR activation by Analog 523 is consistent with our transcriptional profiling in MDA-MB-468 cells (FIGS. 24-35).

For targeted gene expression profiling, Hepa-1 cells were treated with 300 nM of Analog 523 for 24 hours before collecting samples for RNA isolation and cDNA synthesis. cDNA synthesis was performed by converting 3 μg of RNA into cDNA using the Transcriptor First Strand cDNA Synthesis Kit (Roche, Basel, Switzerland). The PAMM-012A Apoptosis PCR Array from SA Biosciences (Qiagen) was used to profile gene expression. Data was analyzed by normalizing Ct values of gene targets to the Ct value for each of the five housekeeping genes, and then taking the average fold change value derived from each normalization.

### IV. Description of AhR Ligand Compounds and Synergizers Therefore

### A. AhR Anti-Cancer Ligand Compounds

The following general formulas are not intended to include impermissible substitution patterns (e.g., methyl substituted with 5 fluoro groups). Such impermissible substitution patterns are known to a person of ordinary skill in the art. Any of the groups referred to herein may be optionally substituted by at least one, possibly two or more, substituents as defined herein. That is, a substituted group has at least one, possible two or more, substitutable hydrogens replaced by a substituent or substituents as defined herein, unless the context indicates otherwise or a particular structural formula precludes substitution.

A person of ordinary skill in the art will appreciate that all of the structural formulas disclosed herein include all tautomers, stereoisomers, hydrates, prodrugs and pharmaceutically acceptable salts thereof.

Additional information concerning AhR ligand compounds according to the present invention is provided by PCT/US2020/044294, published as WO2021022061, which is incorporated herein by reference in its entirety.

Certain embodiments of the present invention concern AhR ligand compounds having a Formula I, shown below.

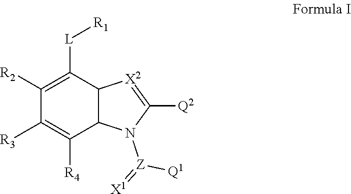

With reference to Formula I:
R¹, R², R³, and R⁴ are independently H, halogen, CN, C1-C10 alkyl, C3-C10 cycloalkyl, C1-C6 alkoxy, SO₂R⁵, CO₂R⁵, or CONR⁵R⁶, or any one of R¹ and R², R² and R³, and R³ and R⁴ pairs, together with the carbon atoms to which they are attached, forms a five- or six-membered cycloalkenyl, heterocyclenyl, aryl, or heteroaryl;
R⁵ and R⁶ are independently H, C1-C10 alkyl, or C3-C10 cycloalkyl, or R⁵ and R⁶, together with the nitrogen atom to which they are attached, form a 5-membered ring or an 6-membered ring.
Q¹ is a C6-C10 aryl; C5-C10 heteroaryl; C5-C10 heterocyclyl; C1-C10 alkyl; or C3-C10 cycloalkyl;
Q² is a C6-C14 aryl; C5-C10 heteroaryl; C5-C10 heterocyclyl; C1-C10 alkyl; or C3-C10 cycloalkyl;
X¹ is absent, or is O, NH, S, or X¹ is

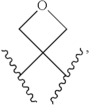 [SMILES: *C1(*)COC1]

where the wavy lines indicate points of attachment to Z;
X² is N, CCl, CF, CBr, CCN, CCONH₂, CCOOH, or CH; and
Z is C, S(O),

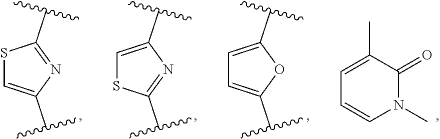

or Z is CH when X¹ is absent.

Certain embodiments of the present invention concern AbR ligand compounds having a formula II, below

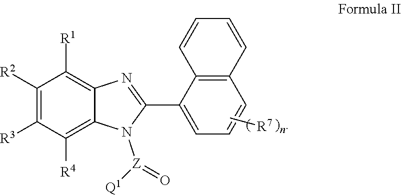

Such compounds are referred to herein generically as naphthalenylbenzoimidazoles. With reference to Formula II:
n is 1, 2, 3, 4, 5, 6 or 7;
R¹, R², R³, and R⁴ are independently H, halogen, CN, C1-C10 alkyl, C3-C10 cycloalkyl, C1-C6 alkoxy, SO₂R⁵, CO₂R⁵, or CONR⁵R⁶, or any one of R¹ and R², R² and R³, and R³ and R⁴ pairs, together with the carbon atoms to which they are attached, forms a five- or six-membered cycloalkenyl, heterocyclenyl, aryl or heteroaryl;
R⁵ and R⁶ are independently H, C1-C10 alkyl, or C3-C10 cycloalkyl, or R⁵ and R⁶, together with the nitrogen atom to which they are attached, form a 5-membered ring or a 6-membered ring;
R⁷, at each occurrence, is independently H, halogen, CN, C1-C10 alkyl, C2-C6 alkenyl, C1-C10 heteroalkyl, C1-C10 heterocyclyl, C6-C10 aryl, C5-C10 heteroaryl, C1-C6 alkoxy, C1-C6 cycloalkyloxy, OCF₃, NR⁵R⁶, SCF₃, or C(O)NR⁵R⁶; and
Q1 is a C6-C10 aryl; C5-C10 heteroaryl; C5-C10 heterocyclyl; C1-C10 alkyl; or C3-C10 cycloalkyl.

Certain embodiments of the present invention concern AhR ligand compounds having a Formula III, shown below

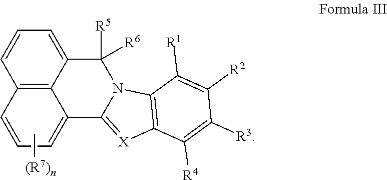

Such compounds are referred to herein generally as benzo[de]benzoimidazoisoquinolines. With reference to Formula III:
n is 1, 2, 3, 4, 5, or 6;
R¹, R², R³, and R⁴ are independently H, halogen, CN, C1-C10 alkyl, C3-C10 cycloalkyl, C1-C6 alkoxy, SO₂R⁸, CO₂R⁸, or CONR⁵R⁹, or any one of R¹ and R², R² and R³, and R³ and R⁴ pairs, together with the carbon atoms to which they are attached, forms a substituted five- or six-membered cycloalkenyl, heterocyclenyl, aryl or heteroaryl ring;
R⁵ and R⁶ are independently H, halogen, OH, or C1-C6 alkyl, or R⁵ and R⁶ taken together are ═O or ═S;
R⁷, at each occurrence, is independently CN, C1-C6 alkyl, or halogen;
R⁸ and R⁹ are H, C1-C10 alkyl, or C3-C10 cycloalkyl, or R⁸ and R⁹, together with the nitrogen atom to which they are attached, form a 5-membered ring or a substituted 6-membered ring; and
X is N or CR¹⁰, where R¹⁰ is s H, halogen, C6-C10 aryl; C5-C10 heteroaryl; or C1-C6 alkyl.

AhR ligand compounds according to the present disclosure may have a Formula IV below

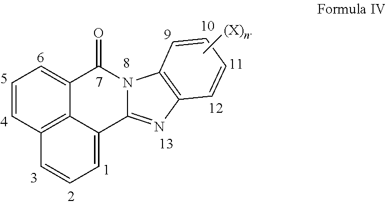

Such compounds are generically referred to herein as benzo[de]benzoimidazoisoquinolinones.

With reference to Formula IV, X is hydrogen or a halogen, that is fluorine, bromine, chlorine or iodine. X can be located on any carbon atom of the phenyl ring, such that n is 0, 1, 2, 3, or 4.

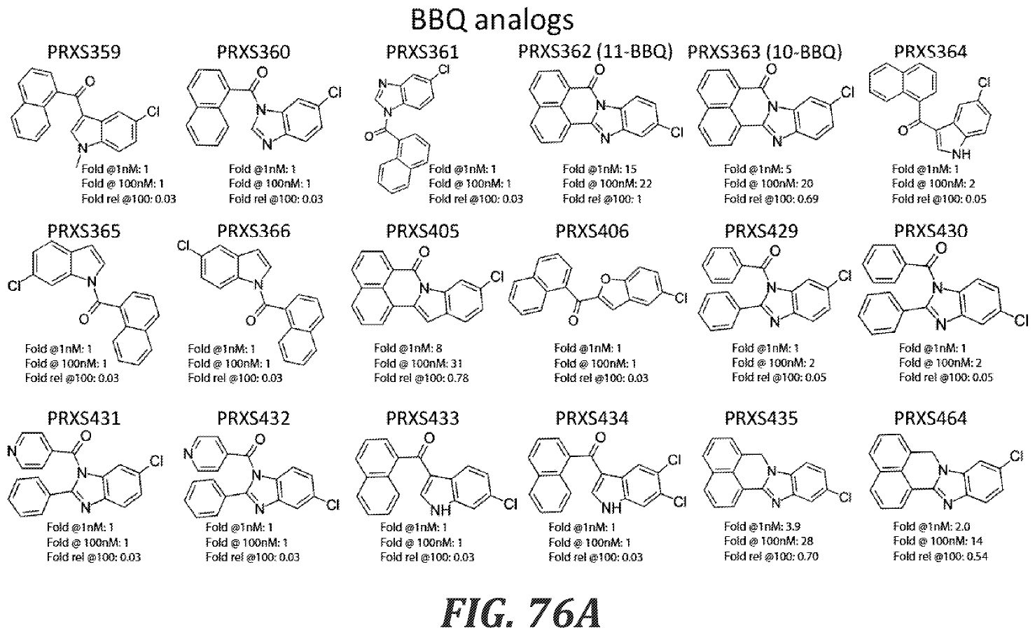

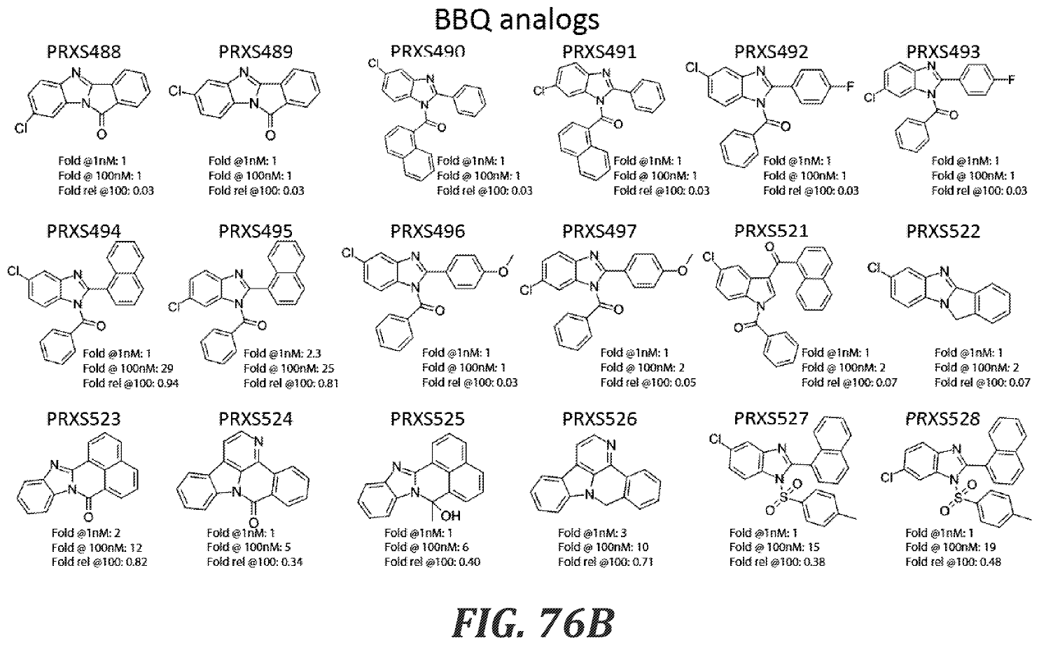

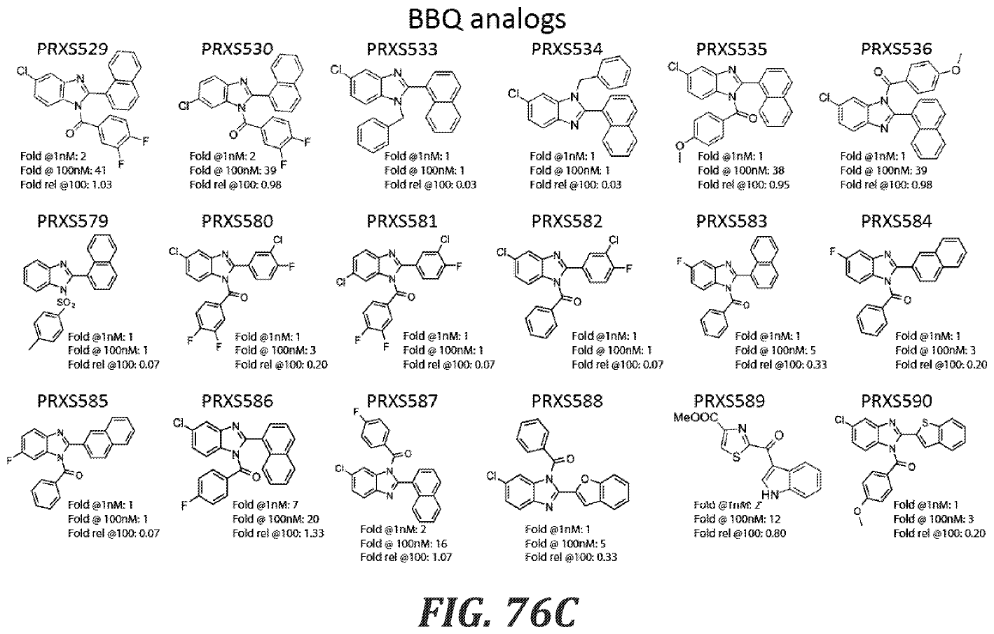

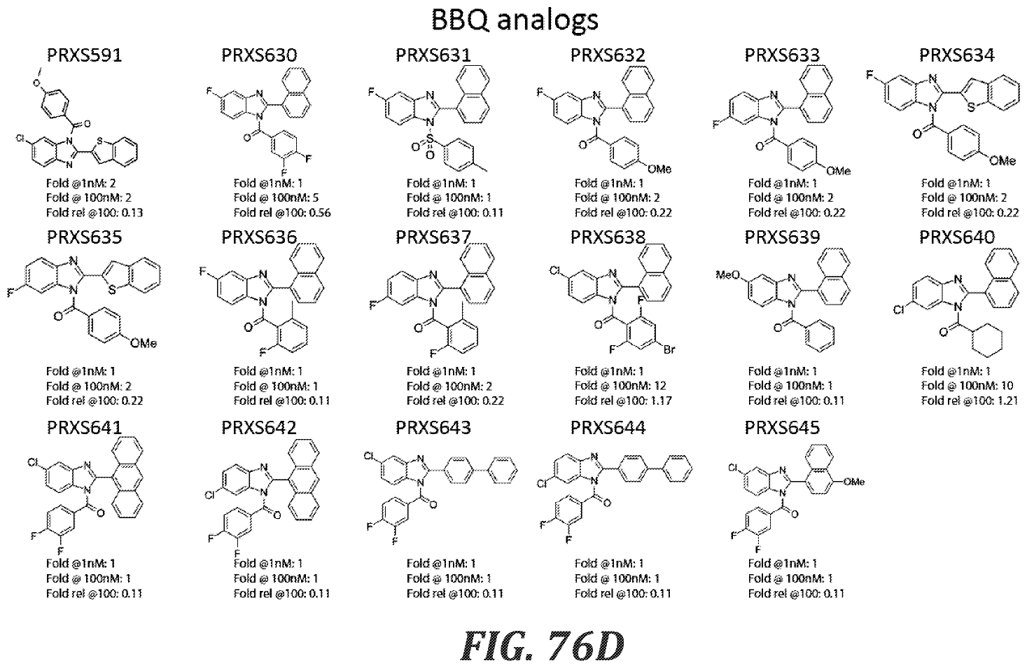

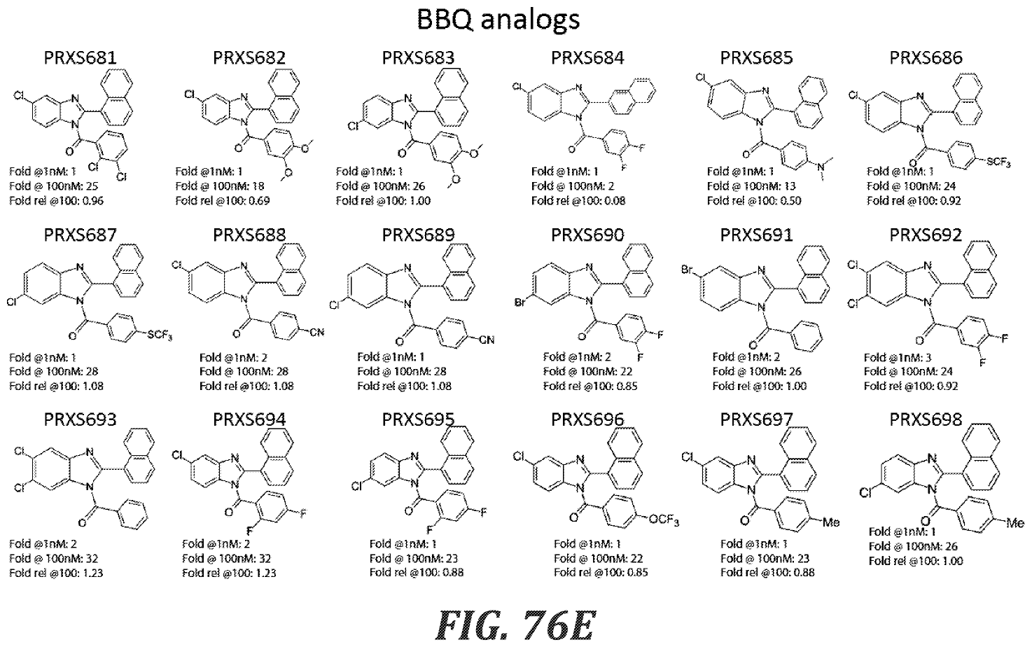

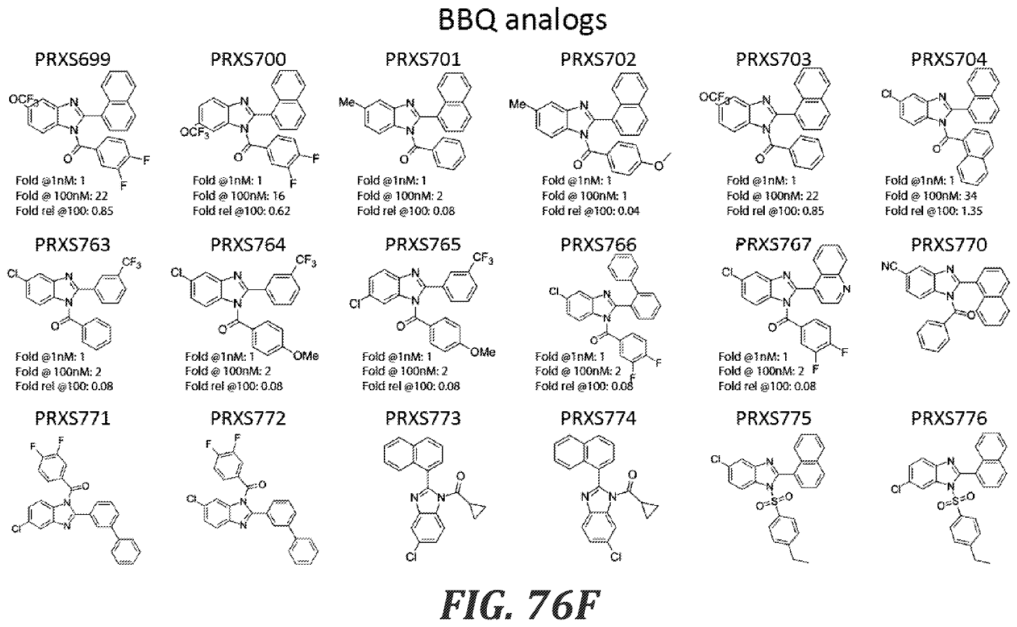

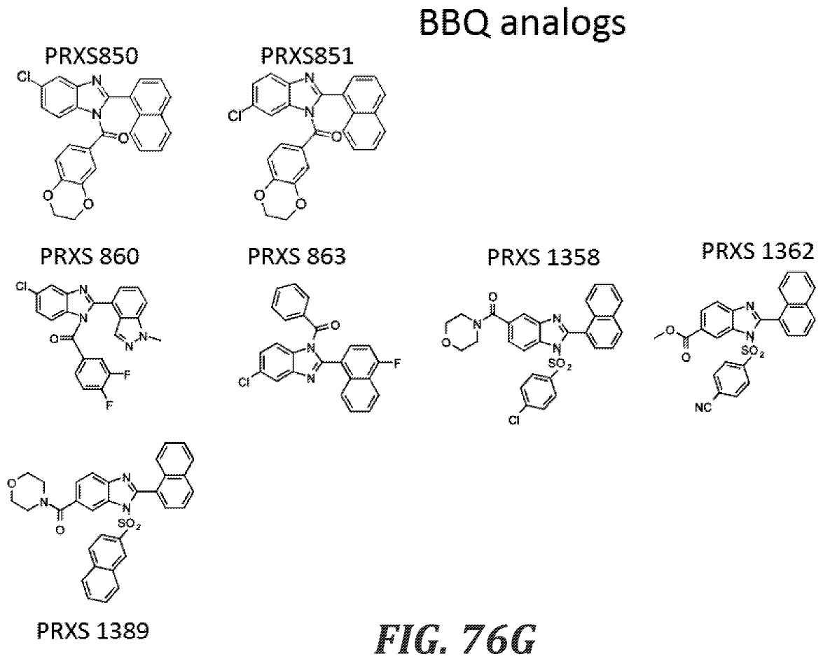

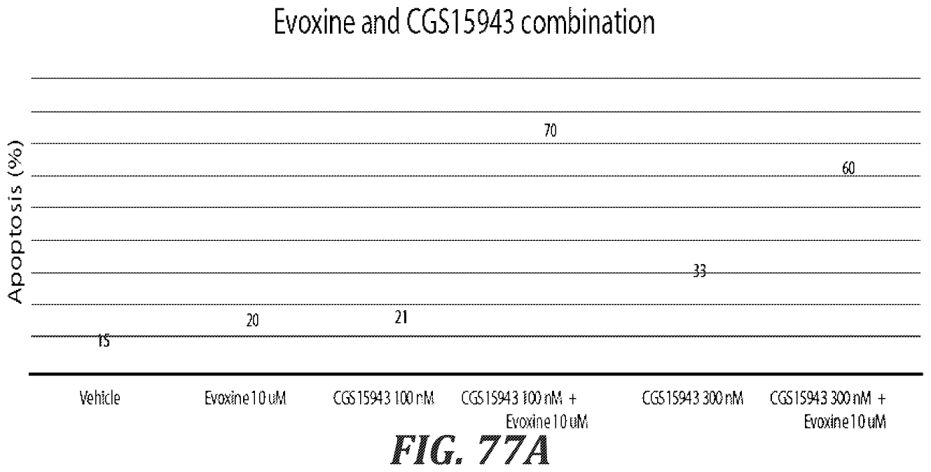

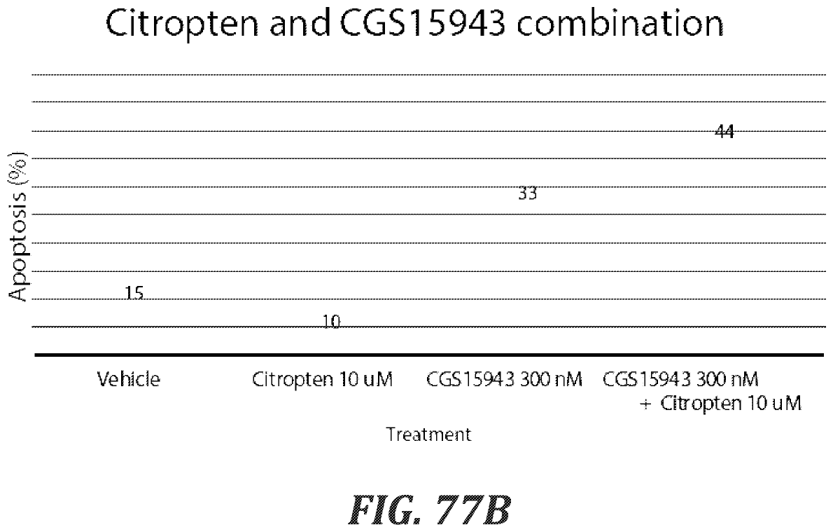

Specific examples of high affinity AbR ligand compounds according to the present invention are presented in FIG. 76.

Currently preferred examples of AhR ligand compounds according to the present invention include BBQ, and 10- or 11-Cl-BBQs.

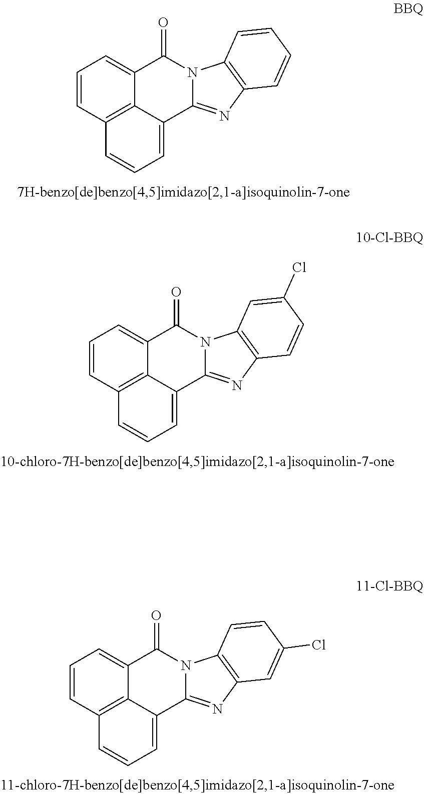 [SMILES: O=c1c2cccc3cccc(c32)c2nc3ccccc3n12 ; O=c1c2cccc3cccc(c32)c2nc3ccc(Cl)cc3n12 ; O=c1c2cccc3cccc(c32)c2nc3cc(Cl)ccc3n12]

### B. Synthesis of Exemplary AhR Ligand Compounds

Information concerning methods for making AbR ligand compounds according to the present disclosure is provided by PCT/US2020/044,294, incorporated herein by reference. Briefly, compounds according to Formula II can be made as shown below in Scheme 1.

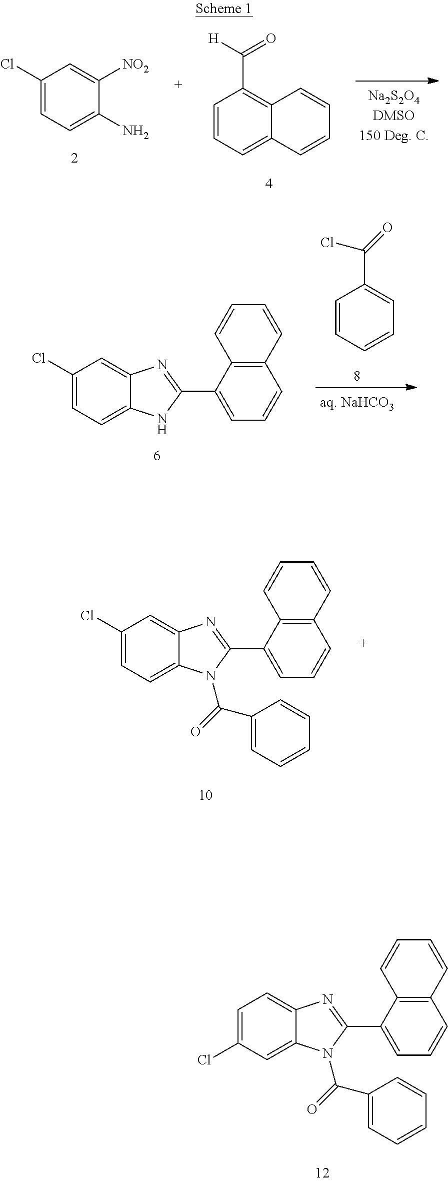 [SMILES: Nc1ccc(Cl)cc1[N+](=O)[O-] ; O=Cc1cccc2ccccc12 ; Clc1ccc2[nH]c(-c3cccc4ccccc34)nc2c1 ; O=C(Cl)c1ccccc1 ; O=C(c1ccccc1)n1c(-c2cccc3ccccc23)nc2cc(Cl)ccc21 ; O=C(c1ccccc1)n1c(-c2cccc3ccccc23)nc2ccc(Cl)cc21]

To a solution of 4-chloro-2-nitroaniline 2 (0.34 g, 2.0 mmol, 1.0 eq) and benzaldehyde 4 (0.21 g, 2.0 mmol, 1.0 eq) in (5.0 mL) in DMSO was added Na₂S₂O₄ (0.61 g, 1.0 mmol, 2.0 eq). The reaction mixture was heated at 150° C. for 3 hours. After completion of reaction, the solution was diluted with water and the precipitate 6 thus obtained was filtered, washed with ether and dried.

To a solution of 6 (0.23 g, 1.0 mmol) in (30 mL) saturated aq NaHCO₃ was added benzoyl chloride 8 (0.13 mL, 1.1 mmol) and the reaction mixture was stirred at room temperature overnight. After completion of reaction, the reaction mixture was diluted with water and extracted with DCM (50 mL×2). The combined organic layer was washed with water (50 mL), brine solution (50 mL), dried over anhydrous sodium sulfate and concentrated under reduced pressure to afford crude products 10 and 12, which were purified by flash chromatography [silica gel 100-200 mesh; elution 0-10% EtOAc in hexane] to afford the compounds as white solids.

Compounds according to Formulas III and IV, such as 10-Cl-BBQ and 11-Cl-BBQ, can be made as shown below in Scheme 2.

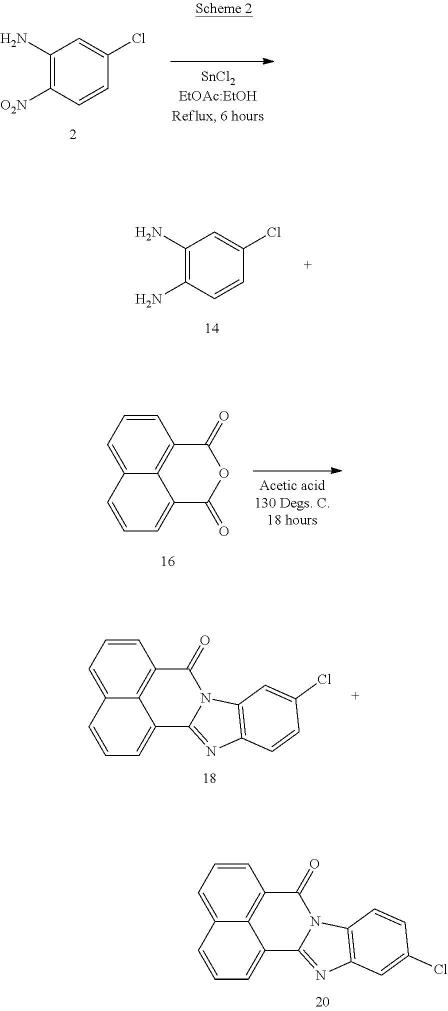 [SMILES: Nc1cc(Cl)ccc1[N+](=O)[O-] ; Nc1ccc(Cl)cc1N ; O=C1OC(=O)c2cccc3cccc1c23 ; O=c1c2cccc3cccc(c32)c2nc3ccc(Cl)cc3n12 ; O=c1c2cccc3cccc(c32)c2nc3cc(Cl)ccc3n12]

To a solution of 5-chloro-2-nitroaniline 2 (10 g, 0.058 mol) in ethyl acetate (30 ml) and ethanol (15 mL) was added tin chloride (54.6 g. 29 mol). The reaction mixture was then refluxed at 80° C. for 16 hours. TLC (30% ethyl acetate in hexane) and NMR showed the formation of desired product 4-chlorobenzene-1,2-diamine 14. The reaction mixture was concentrated under reduced pressure to remove excess solvent and then neutralized with saturated solution of sodium bicarbonate (1000 mL). The reaction mixture was extracted using ethyl acetate (2000 mL). The organic layer was dried over sodium sulphate and concentrated under reduced pressure to afford crude product, which was purified by column chromatography (eluent was 0-30% ethyl acetate in hexane) to obtain 7.0 grams of 4-chlorobenzene-1,2-diamine as an off-white solid.

To a solution of benzo[d]isochromene-1,3-dione 16 (4.10 g, 0.020 mmol) in acetic acid (20 ml) was added 4-chlorobenzene-1,2-diamine 14 (4.01 g. 0.028 mol). The reaction mixture was then heated to 130° C. for 18 hours. LCMS showed the formation of desired product. The reaction mixture was diluted with diethyl ether (50 mL). The precipitate thus obtained was filtered to get crude solid. This solid mass was further triturated in diethyl ether (100 mL) and filtered to get mixture of regioisomers 18 and 20. The crude (mixture of isomers, 1.0 g, 3.27 mmol) was purified by column chromatography.

A person of ordinary skill in the art will appreciate that different species of AhR ligand compounds according to the present invention can be made using Schemes I and II. One such approach would be to change the starting materials, such as compound 2 in Schemes 1 and 2. The chlorine substituent, for example, could be changed for a different halogen, such as F, Br, or I, or could not be included as shown below in Scheme 3, to make BBQ.

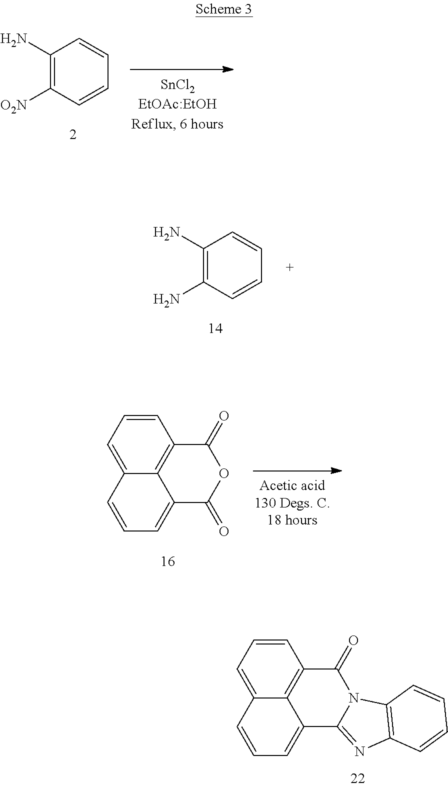 [SMILES: Nc1ccccc1[N+](=O)[O-] ; Nc1ccccc1N ; O=C1OC(=O)c2cccc3cccc1c23 ; O=c1c2cccc3cccc(c32)c2nc3ccccc3n12]

C. Compounds that Provide Synergistic Anti-Cancer Results when used in Combination with AhR Anti-Cancer Ligand Compounds

The presently disclosed embodiments also concern compounds that synergize with AhR anti-cancer ligands. Cancer progression is often driven by numerous molecular alterations that together promote uncontrolled growth. Monotherapies often prove ineffective due to functional redundancy between signaling pathways. As a result, combination therapies, such as those disclosed by the present application, can target multiple signaling modules.

For example, furoquinoline and coumarin derivatives have potent synergistic activity when used in combination with AhR-anti-cancer ligands. Formula V below concerns furoquinoline derivatives that have shown synergistic activity when used in combination with AhR anti-cancer ligands.

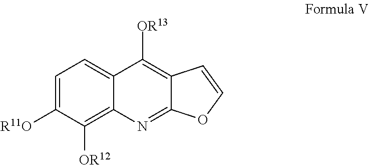

With reference to Formula V, R¹¹ is C1-C10 alkyl, typically C1-C6 alkyl, and even more particularly substituted C1-C10 alkyl, where the substituents are selected from C1-C6 alkyl, hydroxyl, and combinations thereof, with particular examples comprising diols, such as

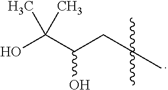 [SMILES: *CC(O)C(C)(C)O]

R¹² and R¹³ also are C1-C6 alkyl, and in particular embodiments R² and R³ are methyl. A particular exemplary compound according to Formula V is provided below.

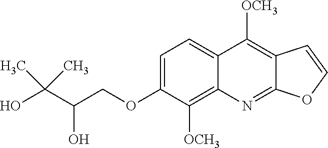 [SMILES: COc1c2ccoc2nc2c(OC)c(OCC(O)C(C)(C)O)ccc12]

And even more particularly the compound is Evoxine having the stereochemistry indicated below.

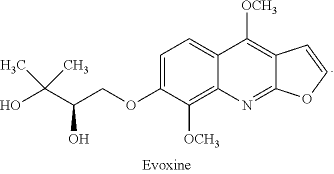 [SMILES: COc1c2ccoc2nc2c(OC)c(OC[C@@H](O)C(C)(C)O)ccc12]

Formula VI, below, concerns coumarin derivatives that have shown synergistic activity when used in combination with AhR anti-cancer ligands.

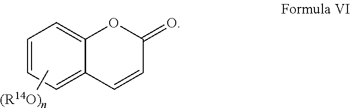

R¹⁴ is C1-C6 alkyl, and n is 1, 2, 3 or 4. In particular embodiments R¹⁴ is methyl, and n=2. One exemplary species within Formula IV, citropten, is provided below.

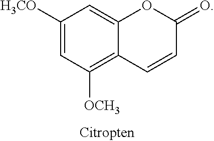 [SMILES: COc1cc(OC)c2ccc(=O)oc2c1]

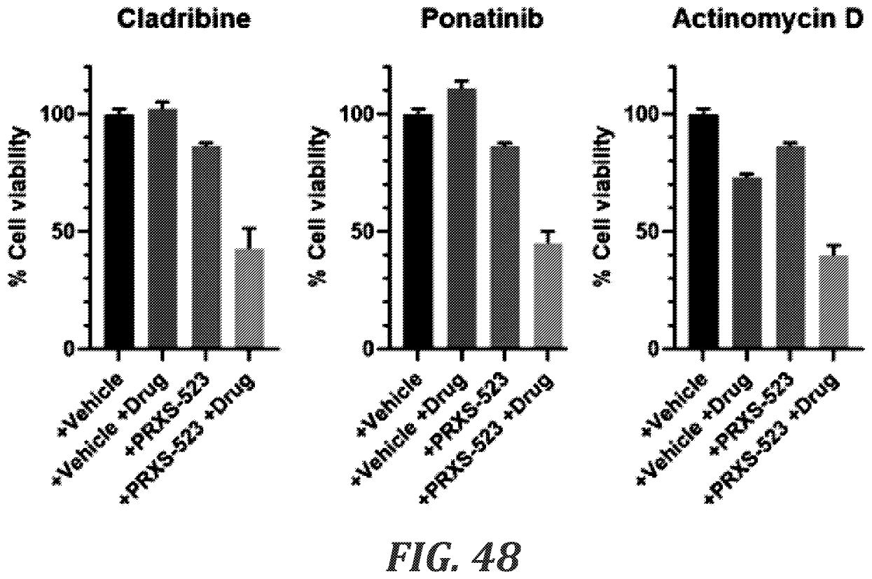

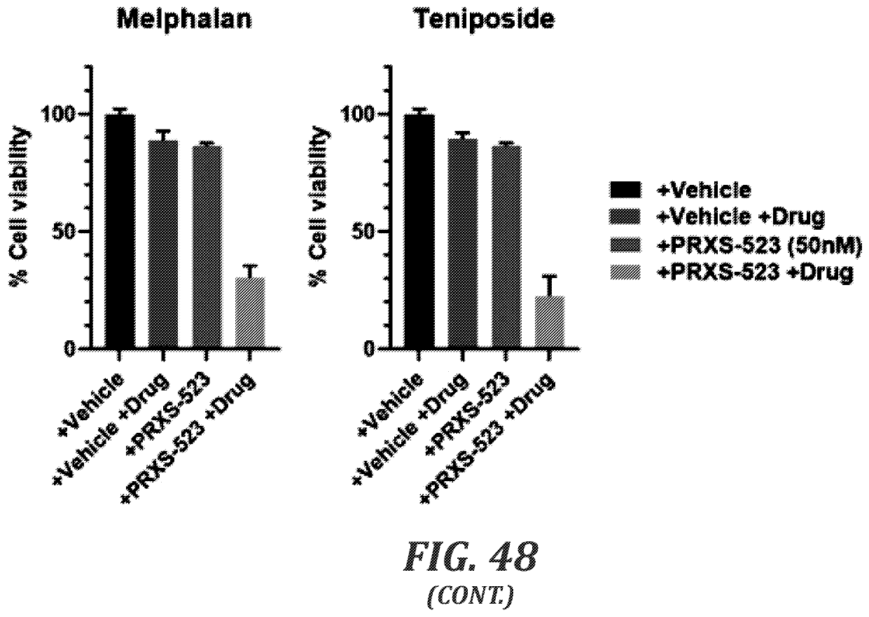

AhR ligand compounds can also be used in combination with other therapeutics, particularly other cancer therapeutics, and such cancer therapeutics may provide synergistic combinations according to the present invention. To date, 120 chemotherapeutics have been screened. Three concentrations of each compound were utilized to co-treat Analog 523 (50 nM), a low concentration that was intentionally selected to have modest effects on its own with shorter exposure time. HDAC inhibitors, nucleoside analogs and proteasome inhibitors are distinct classes of drugs sensitized by AhR ligand compounds according to the present invention. Compounds determined to be effective when used in combination with such AhR ligand compounds, alone or also in combination with synergizers discussed herein with reference to Formulas V and VI, include ponatinib, melphalan, cladribine, mechloroethamine, vorinostat, ibrutinib, actinomycin D, decitabine, fludarabine, cabozantinib, dasatinib, belinostat, clofarabine, gemcitabine, pevonedistat, panobinostat, izaxomib, and teniposide. FIG. 48 provides cell viability data for MDA-MB-468 cells when treated with cladribine, ponatinib, actinomycin D, melphalan, and teniposide alone, in combination with vehicle, treated with Analog 523 alone, and treated with Analog 523 in combination with these exemplary therapeutics.

### V. Synergistic Anti-Cancer Combinations

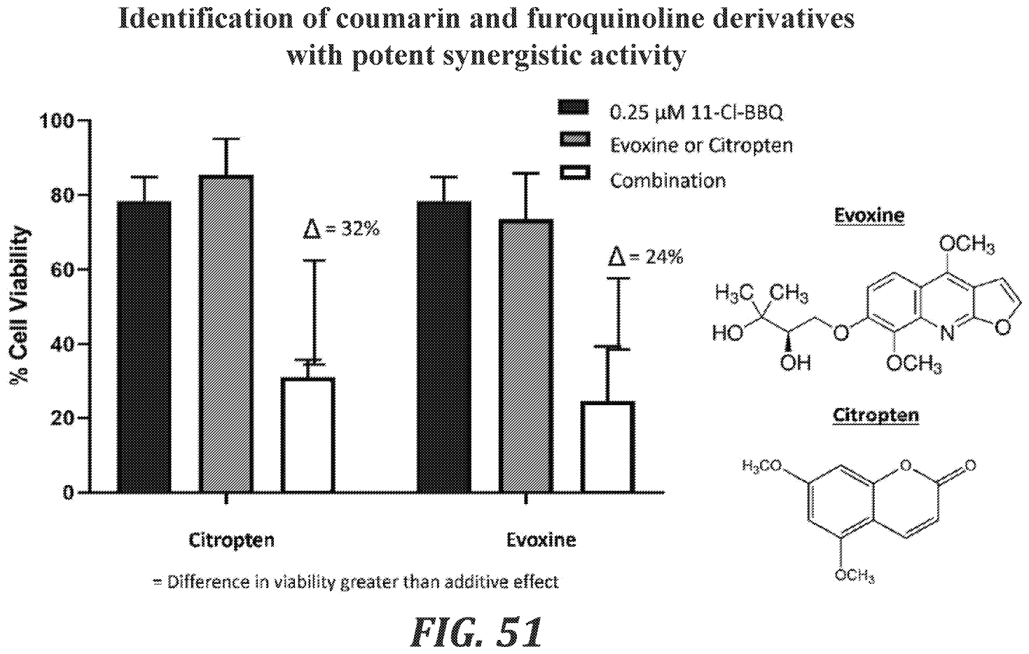

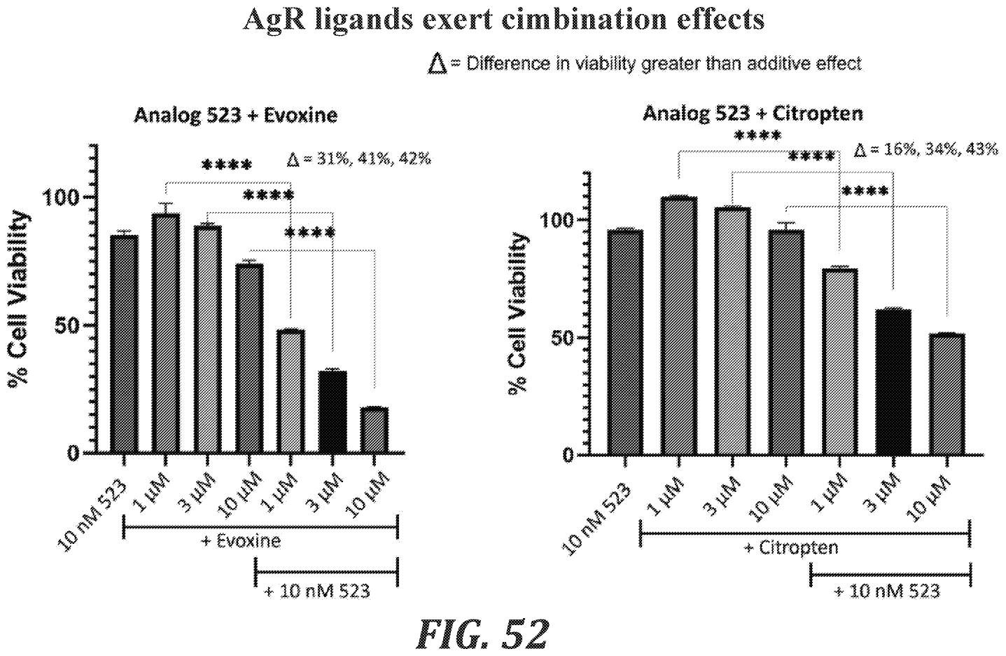

AhR ligand compounds according to Formulas I-IV can be used in combination therapies with compounds according to Formulas V, VI, or other synergizers disclosed herein, and such combinations have been shown to have substantial synergistic anti-cancer activity. FIG. 51 provides data from cancer cell line trials using 11-Cl-BBQ alone, Evoxine alone, Citropten alone, and combinations comprising 11-Cl-BBQ and Citropten and 11-Cl-BBQ and Evoxine. Each of the individual monotreatments showed little effect on the cancer line. Conversely, each of the combination treatments showed substantial and synergistic reductions in cell viability. This also is demonstrated with cell viability studies done with Analog 523 alone, and in combination with Evoxine and in combination with Citropten. This data establishes that the effectiveness is substantially greater than additive, as Analog 523, Evoxine and Citropten, by themselves as mono-applications, were largely ineffective in reducing cell viability. However, the combination treatments substantially reduced cell viability, even when Analog 523 was used at 10 nM, and Evoxine and Citropten were used in amounts of from 1 μM to 10 μM. The decrease in cell viability was more than additive. Specifically, for Evoxine in combination with Analog 523, the reduction in cell viability was: 31% at 1 μM; 41% at 3 μM; and 42% at 10 μM. For Citropten in combination with Analog 523, the reduction in cell viability was: 16% at 1 μM; 34% at 3 μM; and 43% at 10

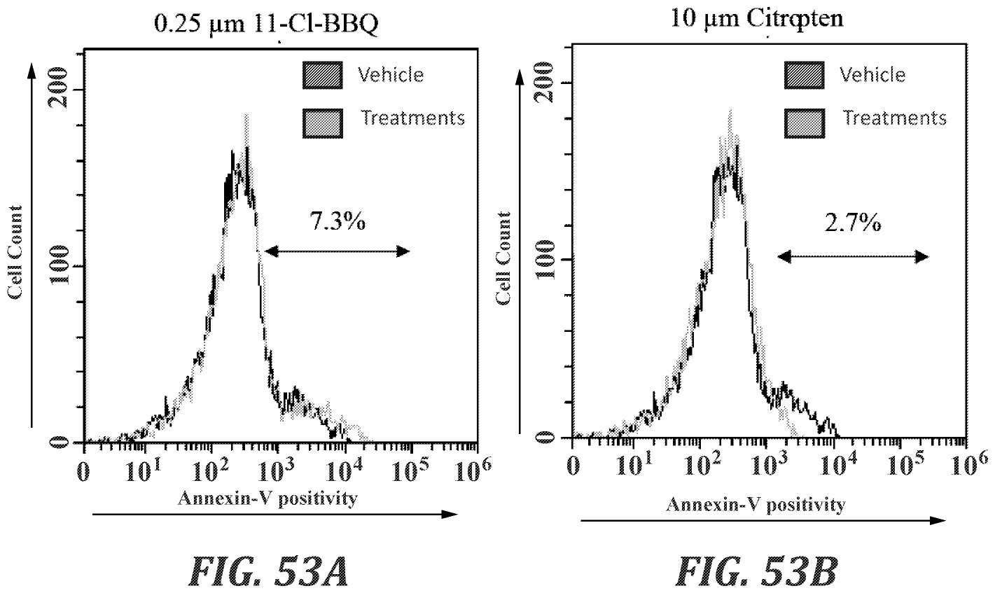

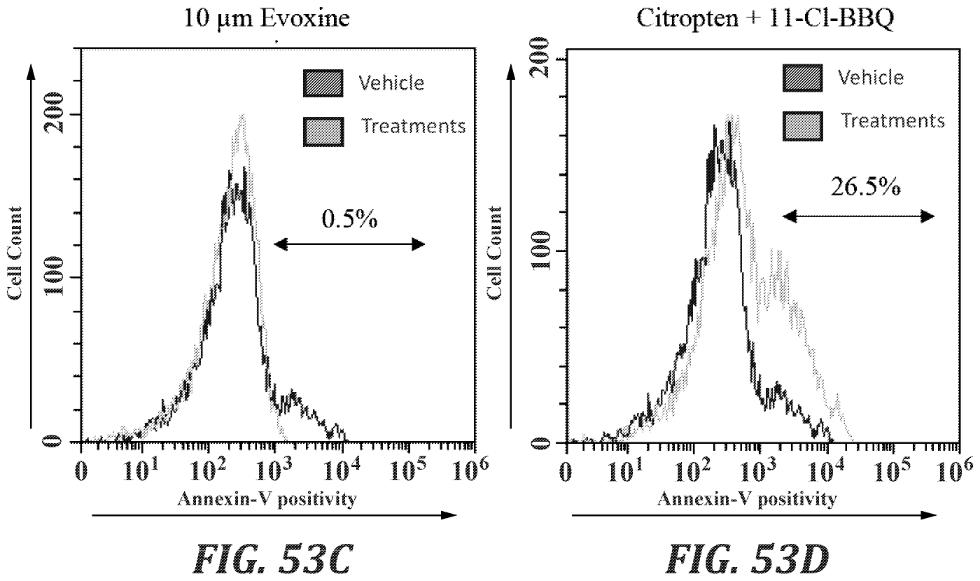

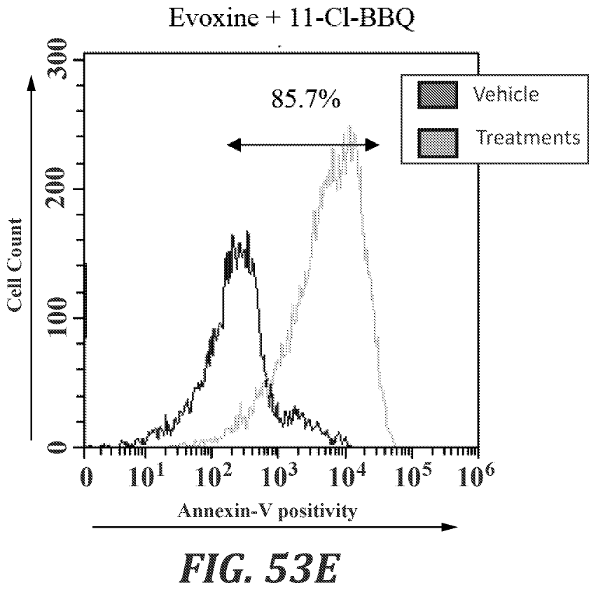

Disclosed combinations have also induced potent apoptotic responses, as established by Annexin-V staining data provided by FIGS. 53A-53E. Again the apoptotic responses are combination dependent. FIGS. 53A-53C show that 11-Cl-BBQ, Citropten and Evoxine evoke very little apoptotic response, ranging from as low as 0.5% for 10 μm Evoxine, to 2.7% for 10 μM Citropten, and 7.3% for 0.25 μM 11-Cl-BBQ. However, when used in combination, the apoptotic response substantially increased to 26.5% for Citropten+11-Cl-BBQ and to 85.7% for Evoxine plus 11-Cl-BBQ.

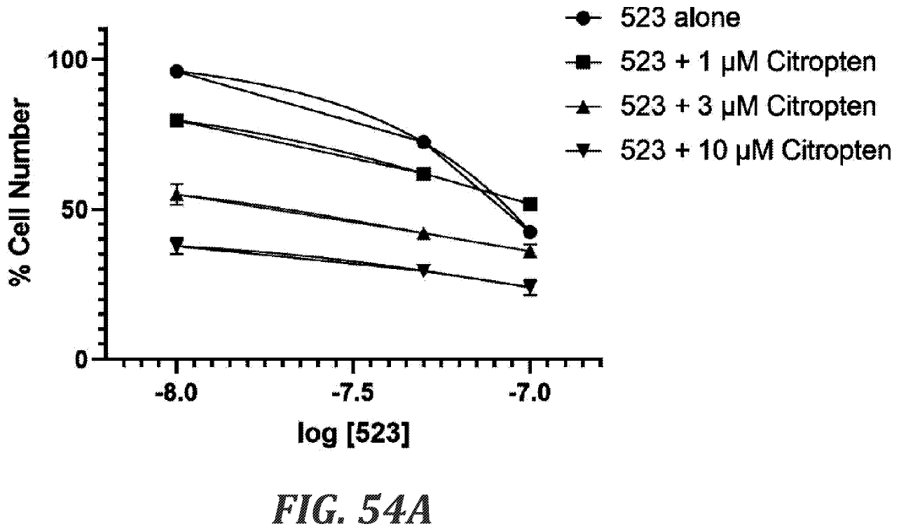

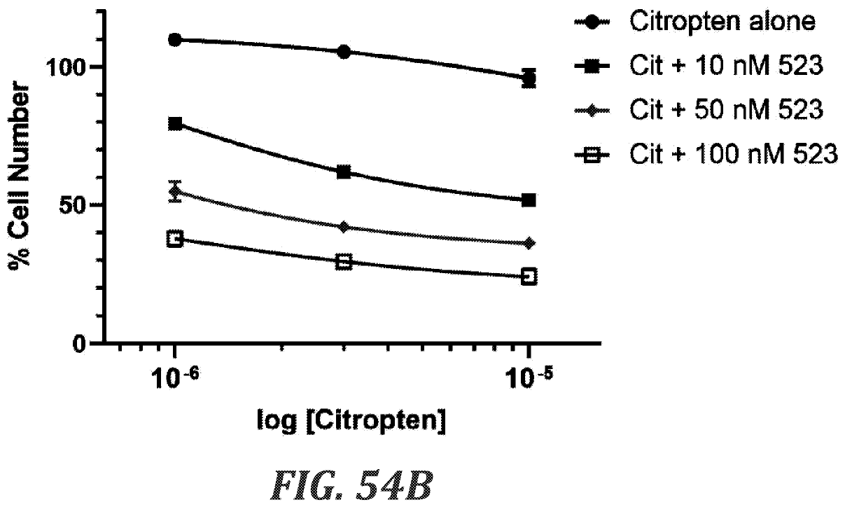

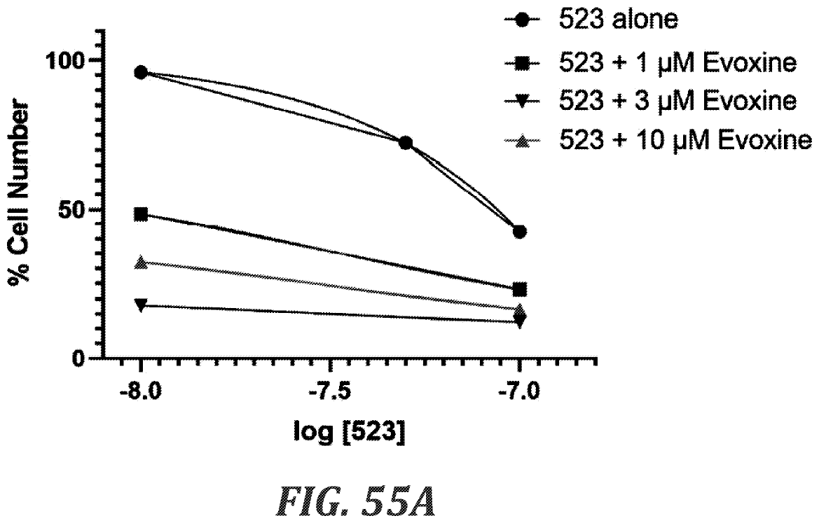

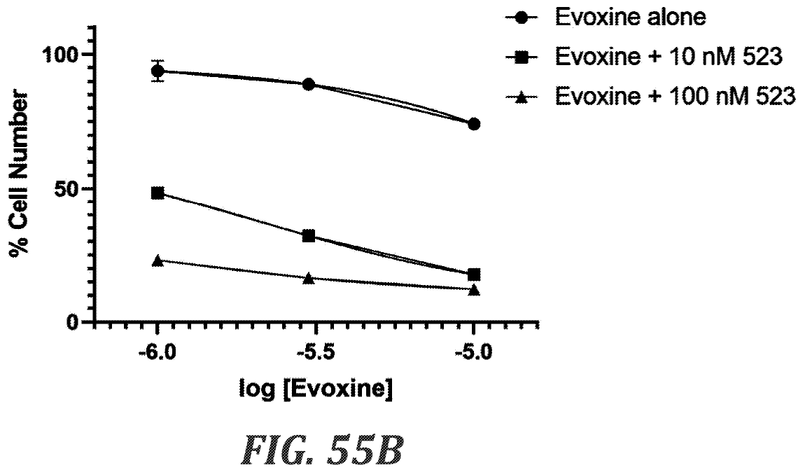

FIGS. 54A,B and FIGS. 55A,B provide concentration dependent results for cell number percentage for Analog 523 alone, Citropten alone or Evoxine alone, versus Analog 523 in combination with Citropten or Evoxine. FIGS. 54A,B and FIGS. 55A,B illustrate the AhR activation required for a combination effect. Specifically, FIGS. 54A, 54B show that Analog 523 by itself has little effect on cell number, until at higher concentrations the overall cytotoxicity of the analog needs to be considered. However, small amounts of Citropten substantially increase the cellular cytotoxicity. Similarly, FIGS. 55A,B show that same effect with Exoxine and Analog 523.

### VI. Formulations Comprising Additional Therapeutics

In some embodiments, combinations comprising Formulas I, II, III, IV, V and/or VI may be administered with another therapeutic agent, such as an analgesic, an antibiotic, an anticoagulant, an antibody, an anti-inflammatory agent, an immunosuppressant, a guanylate cyclase-C agonist, an intestinal secretagogue, an antiviral, anticancer, antifungal, or a combination thereof. The anti-inflammatory agent may be a steroid or a nonsteroidal anti-inflammatory agent. In certain embodiments, the nonsteroidal anti-inflammatory agent is selected from aminosalicylates, cyclooxygenase inhibitors, diclofenac, etodolac, famotidine, fenoprofen, flurbiprofen, ketoprofen, ketorolac, ibuprofen, indomethacin, meclofenamate, mefenamic acid, meloxicam, nambumetone, naproxen, oxaprozin, piroxicam, salsalate, sulindac, tolmetin, or a combination thereof. In some embodiments, the immunosuppressant is mercaptopurine, a corticosteroid, an alkylating agent, a calcineurin inhibitor, an inosine monophosphate dehydrogenase inhibitor, antilymphocyte globulin, antithymocyte globulin, an anti-T-cell antibody, or a combination thereof. In one embodiment, the antibody is infliximab.

In some embodiments, combinations comprising Formulas I, II, III, IV, V and/or VI may be administered with anti-cancer or cytotoxic agents. Various classes of anti-cancer and anti-neoplastic compounds include, but are not limited to, alkylating agents, antimetabolites, BCL-2 inhibitors, vinca alkyloids, taxanes, antibiotics, enzymes, cytokines, platinum coordination complexes, proteasome inhibitors, substituted ureas, kinase inhibitors, hormones and hormone antagonists, and hypomethylating agents, for example DNMT inhibitors, such as azacitidine and decitabine. Exemplary alkylating agents include, without limitation, mechlorothamine, cyclophosphamide, ifosfamide, melphalan, chlorambucil, ethyleneimines, methylmelamines, alkyl sulfonates (e.g., busulfan), and carmustine. Exemplary antimetabolites include, by way of example and not limitation, folic acid analog methotrexate; pyrimidine analog fluorouracil, cytosine arbinoside; purine analogs mercaptopurine, thioguanine, and azathioprine. Exemplary vinca alkyloids include, by way of example and not limitation, vinblastine, vincristine, paclitaxel, and colchicine. Exemplary antibiotics include, by way of example and not limitation, actinomycin D, daunorubicin, and bleomycin. An exemplary enzyme effective as an anti-neoplastic agent includes L-asparaginase. Exemplary coordination compounds include, by way of example and not limitation, cisplatin and carboplatin. Exemplary hormones and hormone related compounds include, by way of example and not limitation, adrenocorticosteroids prednisone and dexamethasone; aromatase inhibitors amino glutethimide, formestane, and anastrozole; progestin compounds hydroxyprogesterone caproate, medroxyprogesterone; and anti-estrogen compound tamoxifen.

### VII. Methods of Using Compounds

### A. Diseases/Disorders

The disclosed compounds, as well as combinations and/or pharmaceutical compositions thereof, may be used to ameliorate, treat or prevent a variety of diseases and/or disorders. In particular embodiments, the disclosed compound, combinations of disclosed compounds, or pharmaceutical compositions thereof, may be useful for treating proliferative disorders.

### B. Formulations and Administration

Pharmaceutical compositions comprising one or more active compounds, combinations and/or compositions of the present disclosure may be manufactured by any suitable method, such as mixing, dissolving, granulating, dragee-making, levigating, emulsifying, encapsulating, entrapping or lyophilization processes. The pharmaceutical compositions may be formulated using one or more physiologically acceptable excipients (e.g., diluents, carriers, or auxiliaries), one or more adjuvants, or combinations thereof, to provide preparations which can be used pharmaceutically.

Pharmaceutical compositions comprising one or more active compounds, combinations and/or compositions of the present disclosure may be formulated using the compounds per se, or in the form of a pharmaceutically acceptable salt, a stereoisomer, an N-oxide, a tautomer, a hydrate, a solvate, an isotope, or a prodrug thereof. Salts may be more soluble in aqueous solutions than the corresponding free acids and bases, but salts having lower solubility than the corresponding free acids and bases may also be formed.

Pharmaceutical compositions of the disclosure may take a form suitable for virtually any mode of administration, including, for example, topical, ocular, oral, buccal, systemic, nasal, injection, such as i.v. or i.p., transdermal, rectal, vaginal, etc., or a form suitable for administration by inhalation or insufflation.

For topical administration, disclosed active compound(s), pharmaceutically acceptable salt, stereoisomer, N-oxide, tautomer, hydrate, solvate, isotope, or prodrug may be formulated as solutions, gels, ointments, creams, suspensions, etc. as are well-known in the art.

Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. Disclosed pharmaceutical compositions may also contain formulating agents, such as suspending, stabilizing and/or dispersing agent. The formulations for injection may be presented in unit dosage form, e.g., in ampules or in multidose containers, and may contain added preservatives.

Alternatively, the injectable formulation may be provided in powder form for reconstitution with a suitable vehicle, including but not limited to sterile, pyrogen-free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients, such as: binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); and/or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable excipients such as: suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the active compound(s).

For buccal administration, the pharmaceutical combinations or compositions may take the form of tablets or lozenges formulated in conventional manner.

For rectal and vaginal routes of administration, the active compound(s) may be formulated as solutions (for retention enemas) suppositories or ointments containing conventional suppository bases, such as cocoa butter or other glycerides.

For nasal administration or administration by inhalation or insufflation, the active compound(s), pharmaceutically acceptable salt, stereoisomer, N-oxide, tautomer, hydrate, solvate, isotope, or prodrug can be conveniently delivered in the form of an aerosol spray from pressurized packs or a nebulizer with the use of a suitable propellant, e.g., dichlorodifluorometbane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

A specific example of an aqueous suspension formulation suitable for nasal administration using commercially available nasal spray devices includes the following ingredients: active compound (0.5 20 mg/ml); benzalkonium chloride (0.1 0.2 mg/mL); polysorbate 80 (TWEEN® 80; 0.5 5 mg/ml); carboxymethylcellulose sodium or microcrystalline cellulose (1-15 mg/ml); phenylethanol (1-4 mg/ml); and dextrose (20-50 mg/ml). The pH of the final suspension can be adjusted to range from about pH 5 to pH 7, with a pH of about pH 5.5 being typical.

Another specific example of an aqueous suspension suitable for administration of the compounds via inhalation contains 20 mg/mL of the disclosed compound(s), 1% (v/v) polysorbate 80 (TWEEN® 80), 50 mM citrate and/or 0.9% sodium chloride.

For ocular administration, the active compound(s) may be formulated as a solution, emulsion, suspension, etc. suitable for administration to the eye. A variety of vehicles suitable for administering compounds to the eye are known in the art. Specific non-limiting examples are described in U.S. Pat. Nos. 6,261,547; 6,197,934; 6,056,950; 5,800,807; 5,776,445; 5,698,219; 5,521,222; 5,403,841; 5,077,033; 4,882,150; and 4,738,851, which are incorporated herein by reference.

For prolonged delivery, the active compound(s) can be formulated as a depot preparation for administration by implantation or intramuscular injection. The active ingredient may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the active compound(s) for percutaneous absorption may be used. To this end, permeation enhancers may be used to facilitate transdermal penetration of the active compound(s). Suitable transdermal patches are described in for example, U.S. Pat. Nos. 5,407,713; 5,352,456; 5,332,213; 5,336,168; 5,290,561; 5,254,346; 5,164,189; 5,163,899; 5,088,977; 5,087,240; 5,008,110; and 4,921,475, which are incorporated herein by reference.

Alternatively, other pharmaceutical delivery systems may be employed. Liposomes and emulsions are well-known examples of delivery vehicles that may be used to deliver active compound(s). Certain organic solvents, such as dimethylsulfoxide (DMSO), may also be employed, although usually at the cost of greater toxicity.

The pharmaceutical compositions may, if desired, be presented in a pack or dispenser device which may contain one or more unit dosage forms containing the active compound(s). The pack may, for example, comprise metal or plastic foil, such as a blister pack. The pack or dispenser device may be accompanied by instructions for administration.

Several approaches exist for transporting molecules across the blood brain barrier. These include, but are not limited to, physical methods, lipid-based methods, and receptor and channel-based methods. Physical methods of transporting a compound across the blood-brain barrier include, but are not limited to, circumventing the blood-brain barrier entirely, and/or creating openings in the blood-brain barrier. Circumvention methods include, but are not limited to, direct injection (e.g., Papanastassiou et al., Gene Therapy 9:398-406, 2002), interstitial infusion/convection enhanced delivery (Bobo et al., Proc. Natl. Acad. Sci. U.S.A. 91:2076-2080, 1994), and implanting a delivery device in the brain (see, e.g., Gill et al., Nature Med. 9:589-595, 2003. Openings in the blood-brain barrier include, but are not limited to, ultrasound, osmotic pressure (e.g., by administration of hypertonic mannitol and permeabilization by, e.g., bradykinin or permeabilizer A-7 (see, e.g., U.S. Pat. Nos. 5,112,596, 5,268,164, 5,506,206, and 5,686,416). Compounds also may be encapsulated in liposomes that are coupled to antibody binding fragments that bind to receptors on the vascular endothelium of the blood-brain barrier.

For certain embodiments, the compounds can be administered continuously by infusion into the fluid reservoirs of the CNS or by bolus injection. Compounds can be administered using an indwelling catheter and a continuous administration means such as a pump, or by implantation of a sustained-release vehicle. For example, the compounds may be injected through chronically implanted cannulas or chronically infused with the help of osmotic minipumps. Subcutaneous pumps can deliver compounds to the cerebral ventricles.

### C. Dosages

The disclosed compound, pharmaceutical compositions, or combinations of disclosed compounds will generally be used in an amount effective to achieve the intended result, for example, in an amount effective to treat, prevent or ameliorate a particular condition, such as a proliferative disease. Therapeutic benefit means eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. For example, administration of a compound to a patient suffering from an allergy provides therapeutic benefit not only when the underlying allergic response is eradicated or ameliorated, but also when the patient reports a decrease in the severity or duration of the symptoms associated with the allergy following exposure to the allergen. As another example, therapeutic benefit in the context of asthma includes an improvement in respiration following the onset of an asthmatic attack or a reduction in the frequency or severity of asthmatic episodes. Therapeutic benefit also includes halting or slowing the progression of the disease, regardless of whether improvement is realized.

As known by those of ordinary skill in the art, the preferred dosage of disclosed compounds may depend on various factors, including the age, weight, general health, and severity of the condition of the patient or subject being treated. Dosage also may need to be tailored to the sex of the individual and/or the lung capacity of the individual, when administered by inhalation. Dosage may also be tailored to individuals suffering from more than one condition or those individuals who have additional conditions that affect lung capacity and the ability to breathe normally, for example, emphysema, bronchitis, pneumonia, respiratory distress syndrome, chronic obstructive pulmonary disease, and respiratory infections. A person of ordinary skill in the art will be able to determine the optimal dose for a particular individual.

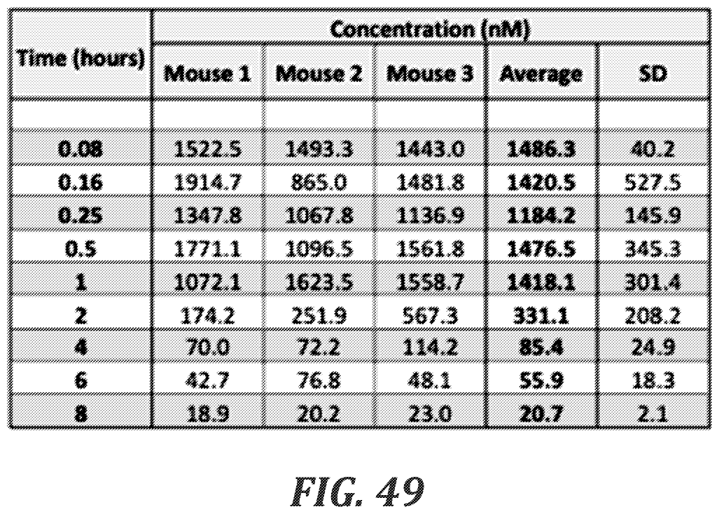

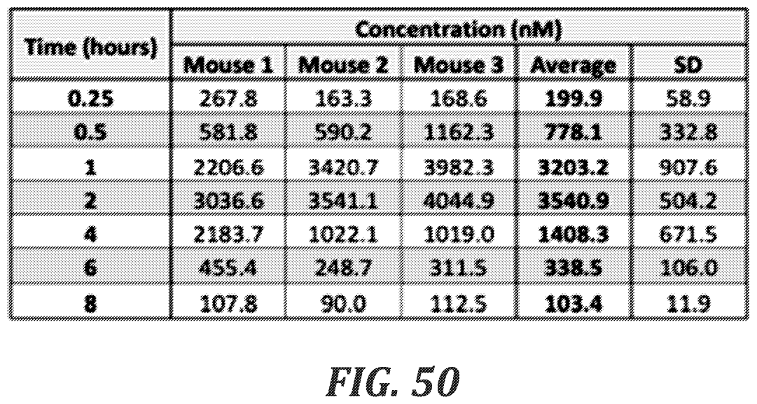

Effective dosages can be estimated initially from in vitro or in vivo assays. For example, an initial dosage for use in subjects can be formulated to achieve a desired circulating blood or serum concentration of active compound. See, for example, FIGS. 49 and 50. Dosages can be calculated to achieve such circulating blood or serum concentrations taking into account the bioavailability of the particular compound. Fingl & Woodbury, “General Principles,” In: Goodman and Gilman's The Pharmaceutical Basis of Therapeutics, Chapter 1, pages 1-46, Pergamon Press, and the references cited therein, provide additional guidance concerning effective dosages.

In some embodiments, the disclosed AhR ligand compounds have a biological effect when administered in amounts ranging from 1 pM to 100 μM, such as 1 pM to 10 μM, or 1 pM to 100 nM. Synergizers for use in combination with disclosed AhR ligand compounds typically are used in micromolar concentrations, such as from 100 nM to 100 μM, more typically 3 μM to 50 μM.

Initial dosages can also be estimated from in vivo data, such as animal models, again as demonstrated by FIGS. 49 and 50. Animal models useful for testing the efficacy of compounds to treat or prevent the various diseases described above are well-known in the art. Persons of ordinary skill in the art can adapt such information to determine dosages suitable for human administration.

Dosage amounts of disclosed compounds will typically be in the range of from greater than 0 mg/kg/day, such as 0.0001 mg/kg/day or 0.001 mg/kg/day or 0.01 mg/kg/day, up to at least about 100 mg/kg/day. More typically, the dosage (or effective amount) may range from about 0.0025 mg/kg to about 1 mg/kg administered at least once per day, such as from 0.01 mg/kg to about 0.5 mg/kg or from about 0.05 mg/kg to about 0.15 mg/kg. The total daily dosage typically ranges from about 0.1 mg/kg to about 5 mg/kg or to about 20 mg/kg per day, such as from 0.5 mg/kg to about 10 mg/kg per day or from about 0.7 mg/kg per day to about 2.5 mg/kg/day. Dosage amounts can be higher or lower depending upon, among other factors, the activity of the disclosed compound, its bioavailability, the mode of administration, and various factors discussed above.

Dosage amount and dosage interval can be adjusted for individuals to provide plasma levels of the disclosed compound that are sufficient to maintain therapeutic or prophylactic effect. For example, the compounds can be administered once per day, multiple times per day, once per week, multiple times per week (e.g., every other day), one per month, multiple times per month, or once per year, depending upon, amongst other things, the mode of administration, the specific indication being treated, and the judgment of the prescribing physician. Persons of ordinary skill in the art will be able to optimize effective local dosages without undue experimentation.

Pharmaceutical compositions comprising one or more of the disclosed compounds typically comprise from greater than 0 up to 99% of the disclosed compound, or compounds, and/or other therapeutic agent by total weight percent. More typically, pharmaceutical compositions comprising one or more of the disclosed compounds comprise from about 1 to about 20 total weight percent of the disclosed compound and other therapeutic agent, and from about 80 to about 99 weight percent of a pharmaceutically acceptable excipient. In some embodiments, the pharmaceutical composition can further comprise an adjuvant.

Preferably, the disclosed compound, combinations of disclosed compounds, or pharmaceutical compositions thereof, will provide therapeutic or prophylactic benefit without causing substantial toxicity. Toxicity of the disclosed compound can be determined using standard pharmaceutical procedures. The dose ratio between toxic and therapeutic (or prophylactic) effect is the therapeutic index. Disclosed compounds that exhibit high therapeutic indices are preferred.

### VIII. Cancers Treatable with Disclosed Combinations

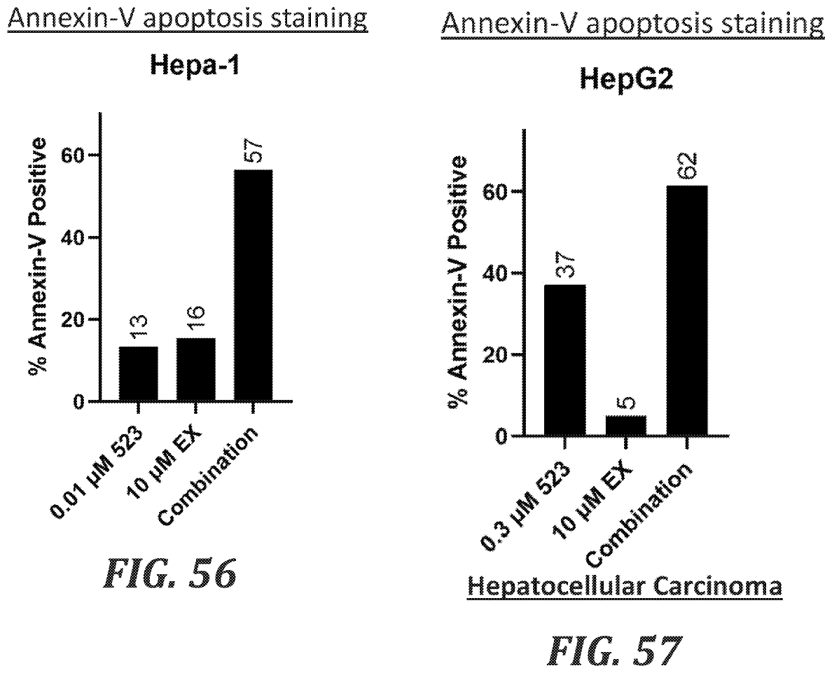

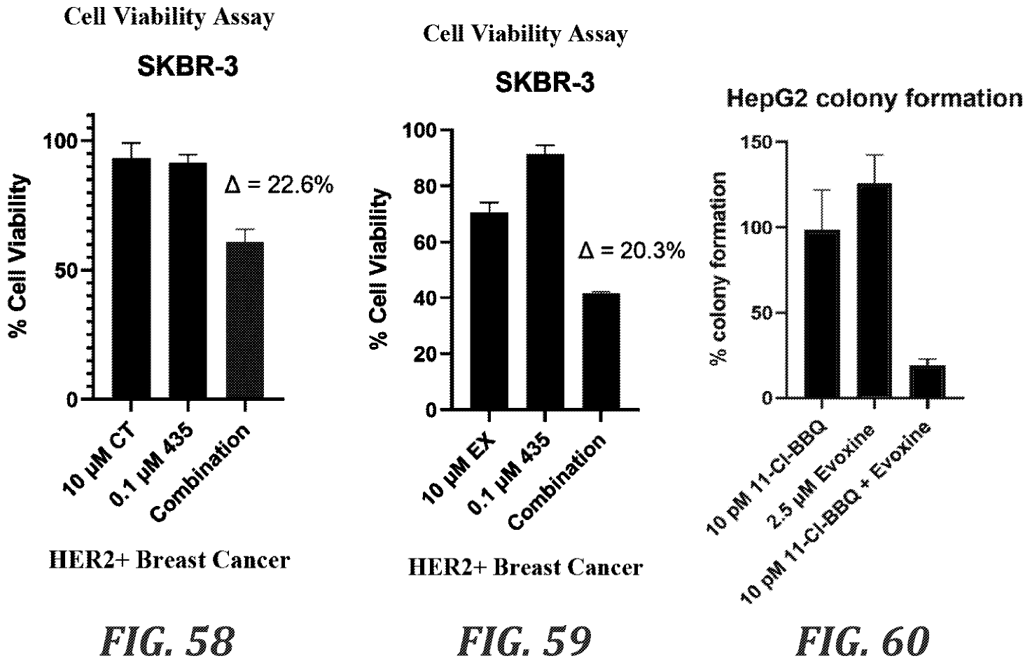

FIGS. 56-59 provide data establishing that combinations disclosed herein are effective for treating cancerous cells generally. FIGS. 56-57 provide Annexin-V staining for hepatoma (mouse) and hepatocellular carcinoma (mouse). FIGS. 58 and 59 provide cell viability assays for HER2+ breast cancer. Specifically, FIG. 56 provides Annexin-V apoptosis staining data for 0.01 μM analog 523 alone, 10 μM Evoxine alone, and 0.01 μM analog 523+10 μM Evoxine showing a 24% increase in apoptotic response for the combination 0.01 μM analog 523+Evoxine for Hepatoma (mouse) cells. FIG. 57 provides Annexin-V apoptosis staining data for 0.01 μM analog 523 alone, Evoxine alone, and 0.01 μM analog 523+10 μM Evoxine showing a 20% increase in apoptotic response for the combination 0.01 μM analog 523+Evoxine for Hepatoma (mouse) cells. FIG. 58 provides cell viability data for 10 μM Citropten, 0.1 μM analog 435, shown below, and the combination of 10 μM Citropten+0.1 μM analog 435 for SKBR-3 HER2+ breast cancer cells showing a 22.6% decrease in cell viability.

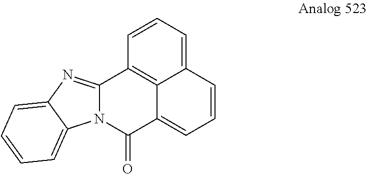 [SMILES: O=c1c2cccc3cccc(c32)c2nc3ccccc3n12]

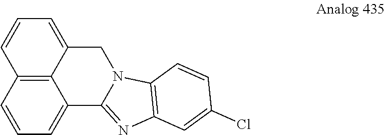 [SMILES: Clc1ccc2c(c1)nc1n2Cc2cccc3cccc-1c23]

FIG. 59 provides cell viability data for 10 μM Evoxine, 0.1 μM analog 435, and the combination of 10 μM Evoxine+0.1 μM analog 435 for SKBR-3 HER2+ breast cancer cells showing a 20.3% decrease in cell viability.

The inhibition of HepG2 colony formation provides additional data supporting efficacy of disclosed combinations. Specifically, FIG. 60 is a bar graph showing colony formation (%) versus treatment, namely 10 μM 11-Cl-BBQ, 2.5 μM Evoxine and 10 μM 11-Cl-BBQ+2.5 μM Evoxine. FIG. 60 shows that 10 μM 11-Cl-BBQ and 2.5 μM Evoxine had little effect on colony formation inhibition. However, the combination of 10 μM 11-Cl-BBQ and 2.5 μM Evoxine virtually eradicated HepG2 colony formation. Thus, concentrations of the AhR ligand, such as those according to Formulas I-IV, as low as picomolar, in combination with low micromolar concentrations of compounds according to Formulas V, VI, ponatinib, melphalan, cladribine, mechloroethamine, vorinostat, ibrutinib, actinomycin D, decitabine, fludarabine, cabozantinib, dasatinib, belinostat, clofarabine, gemcitabine, pevonedistat, panobinostat, izaxomib, and/or teniposide, substantially preclude HepG2 colony formation.

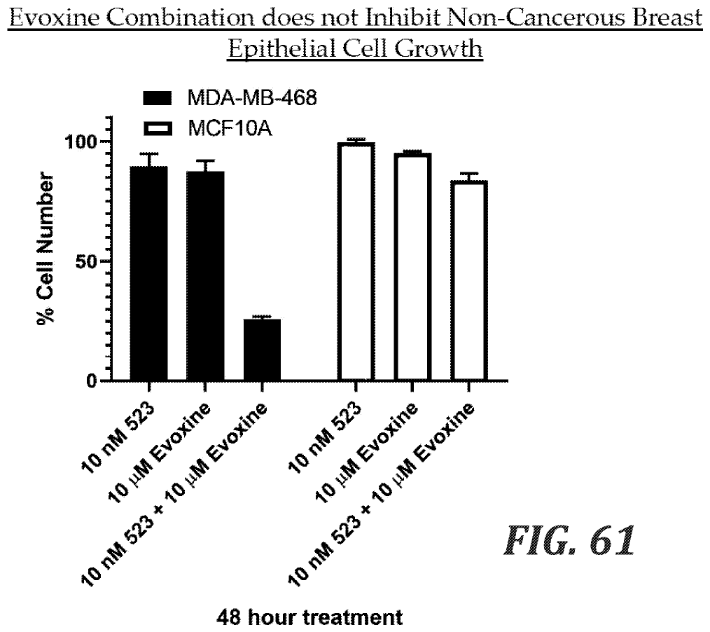

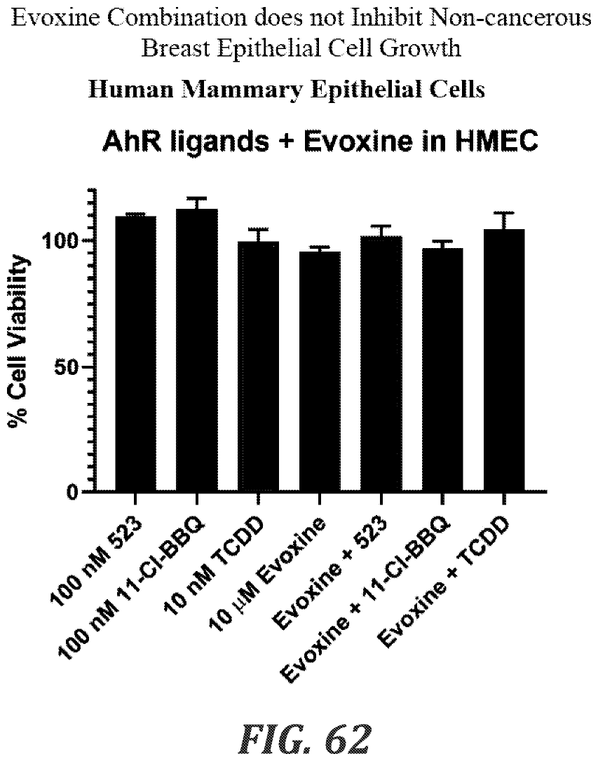

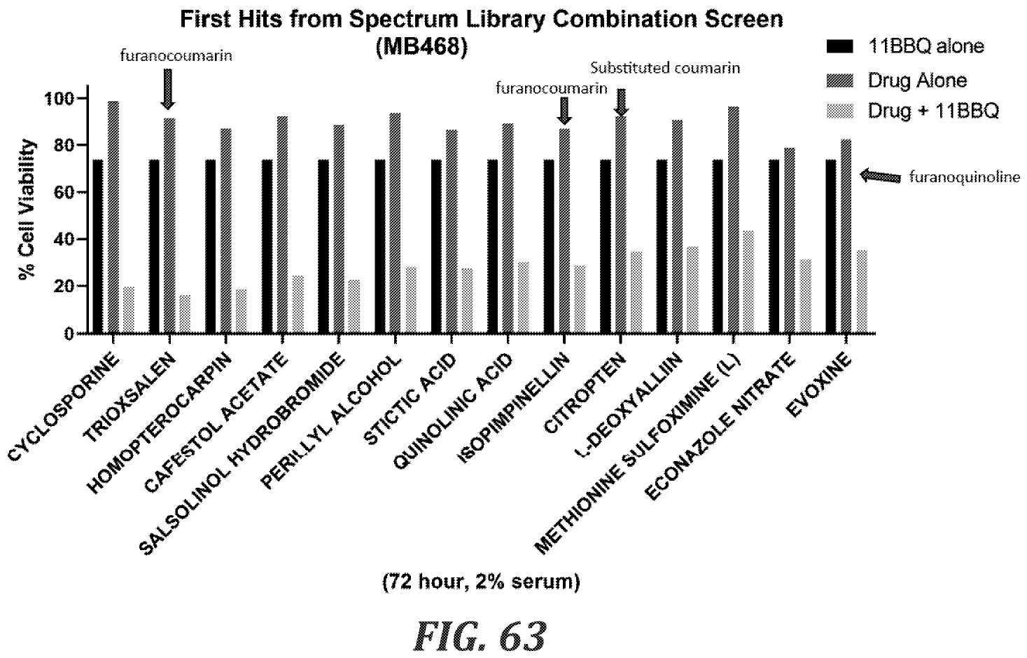

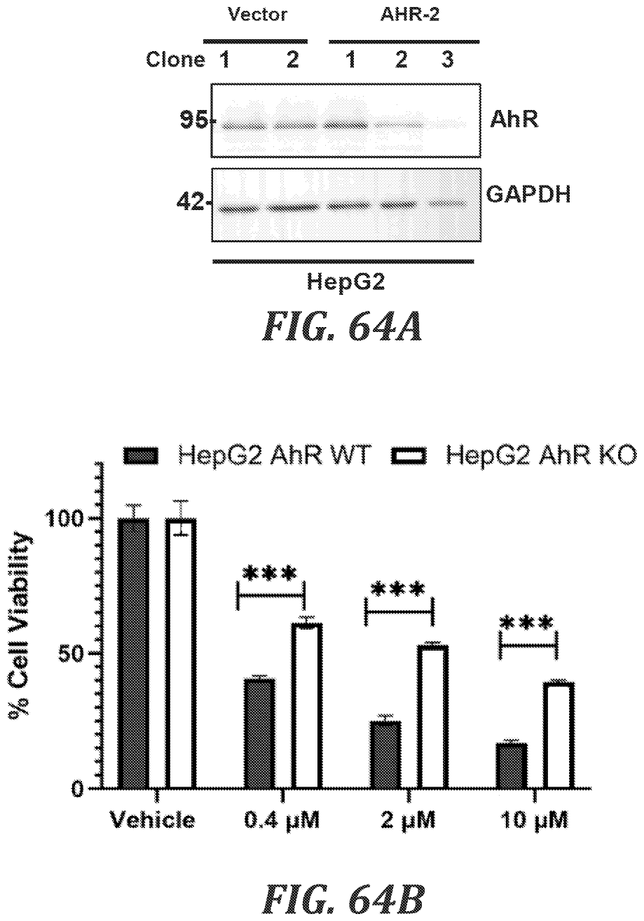

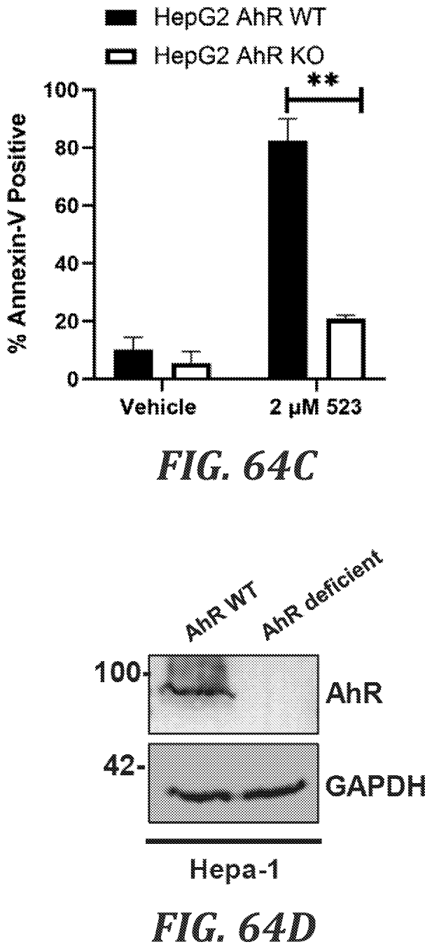

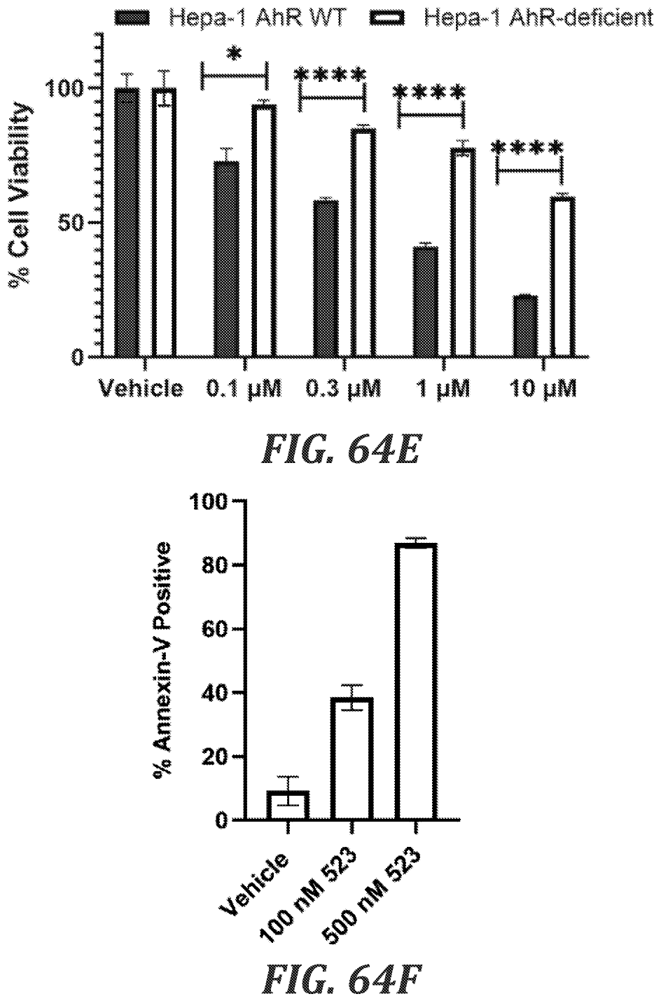

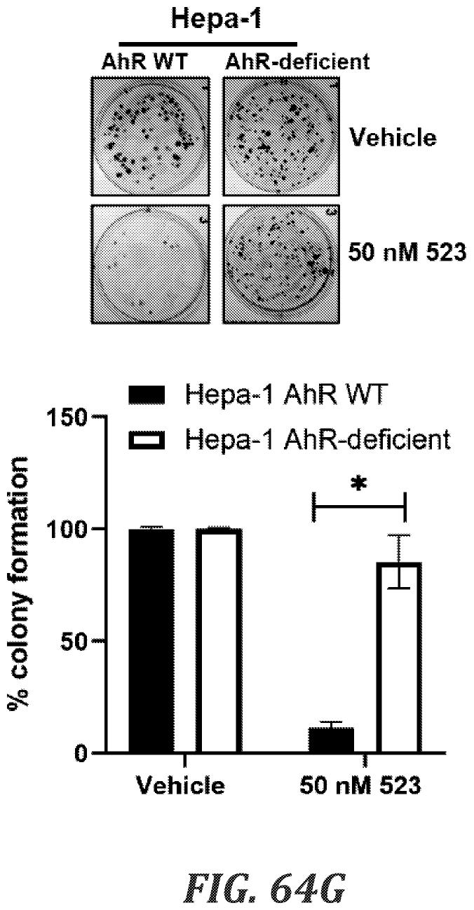

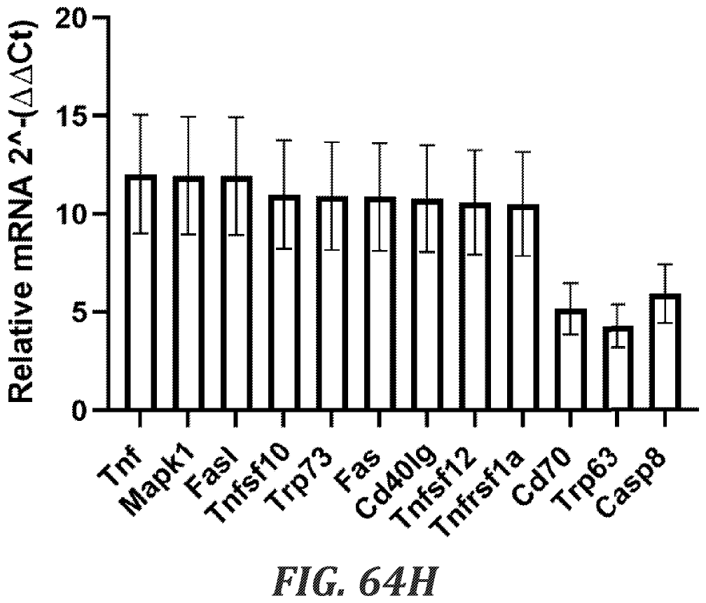

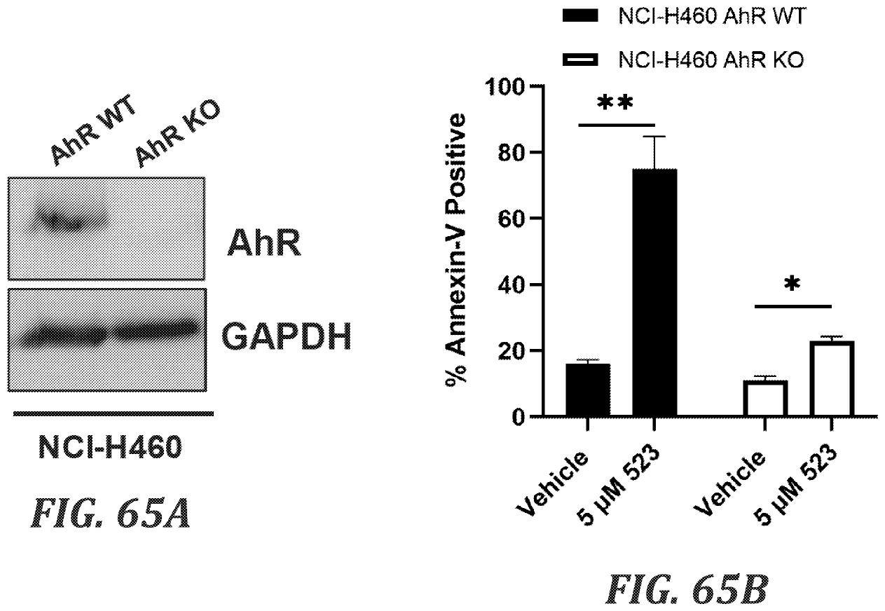

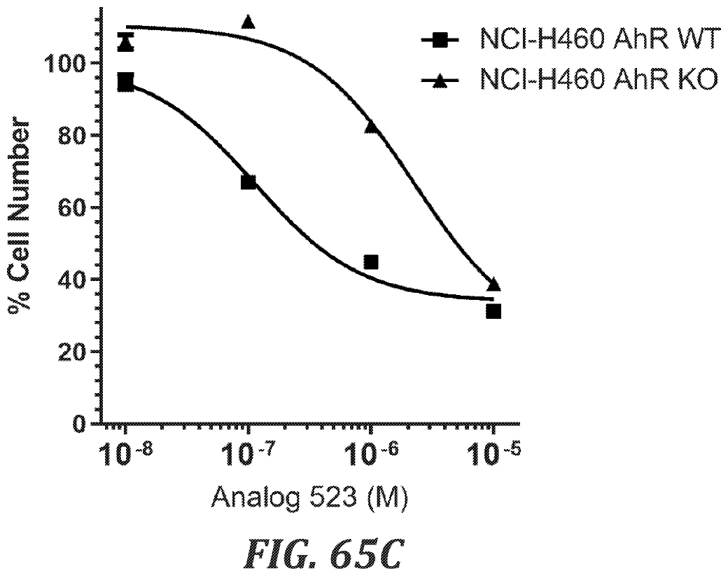

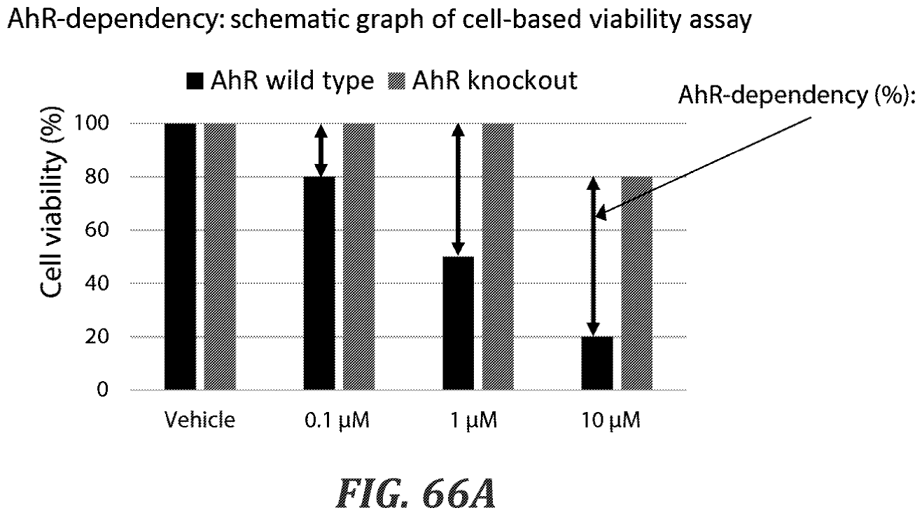

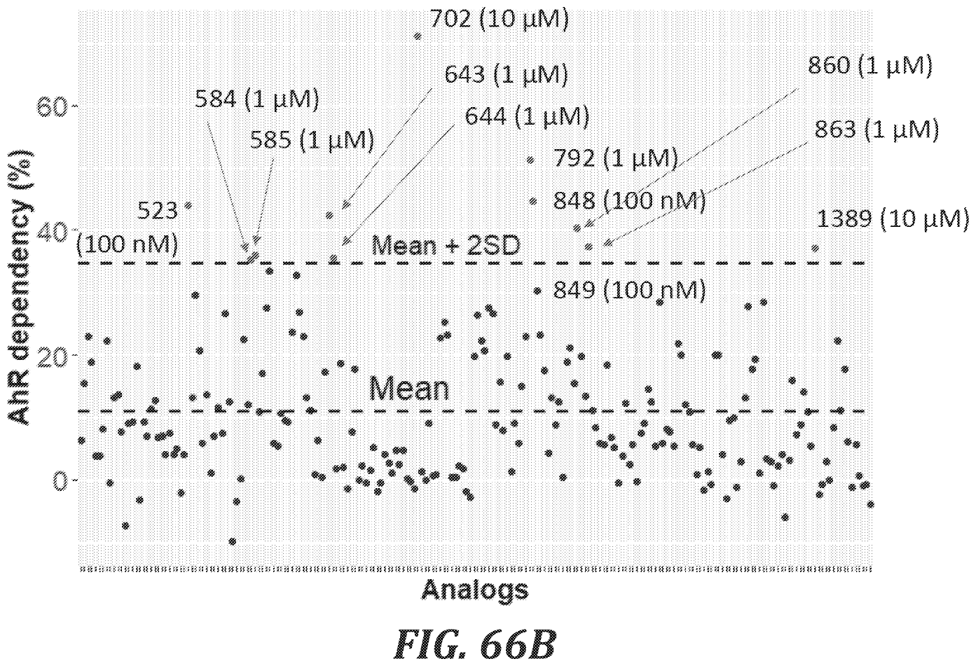

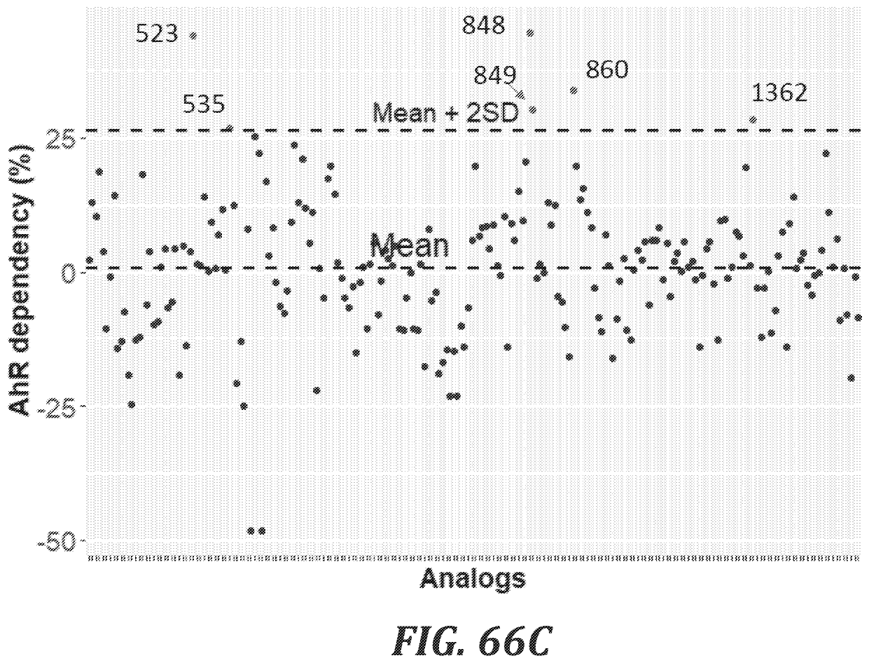

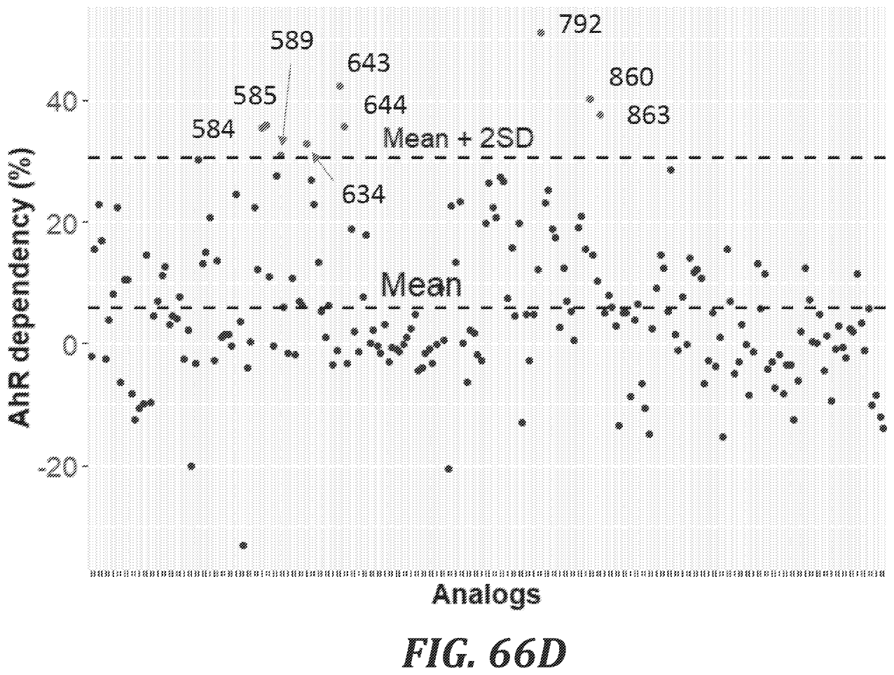

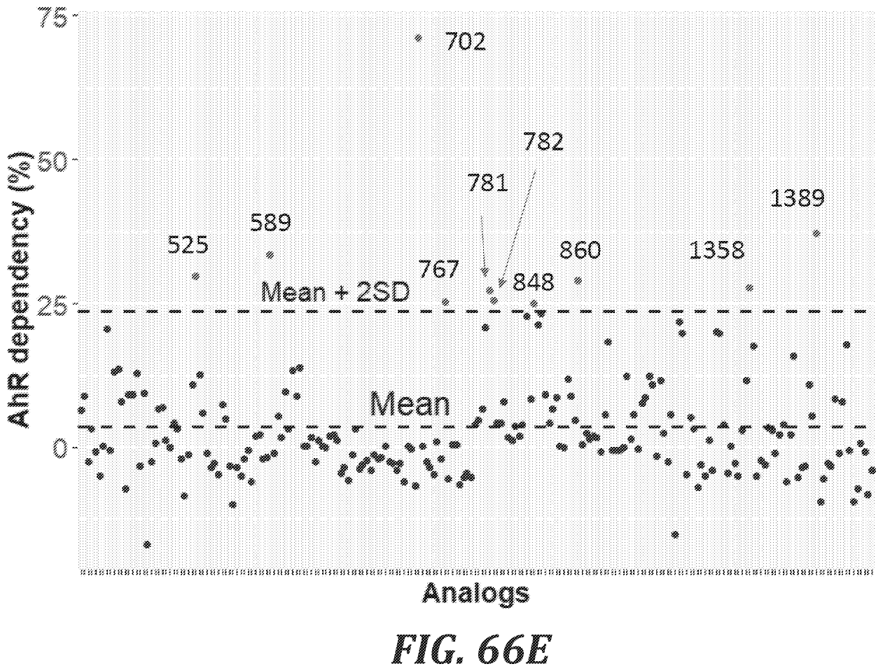

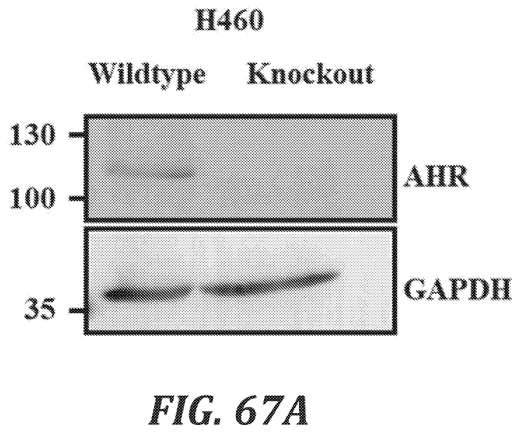

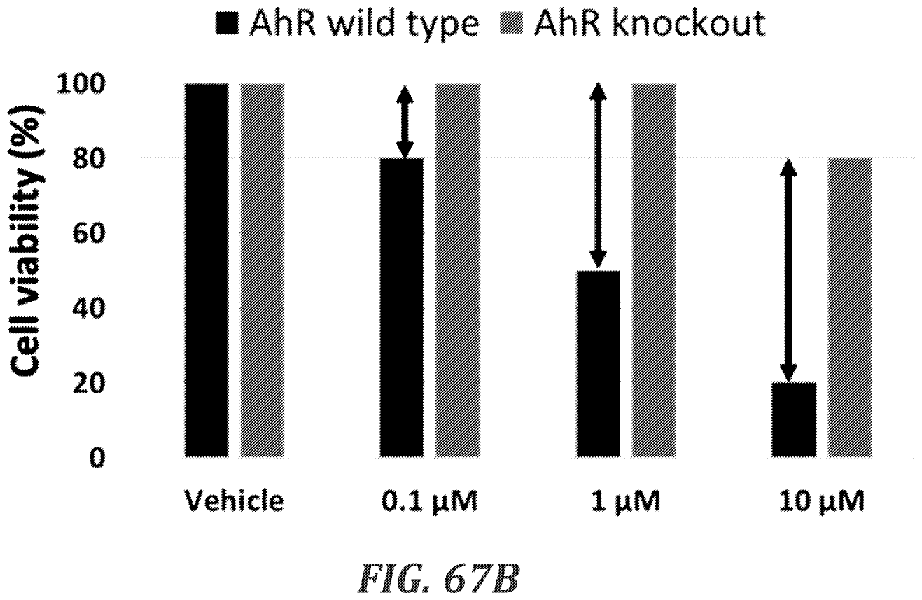

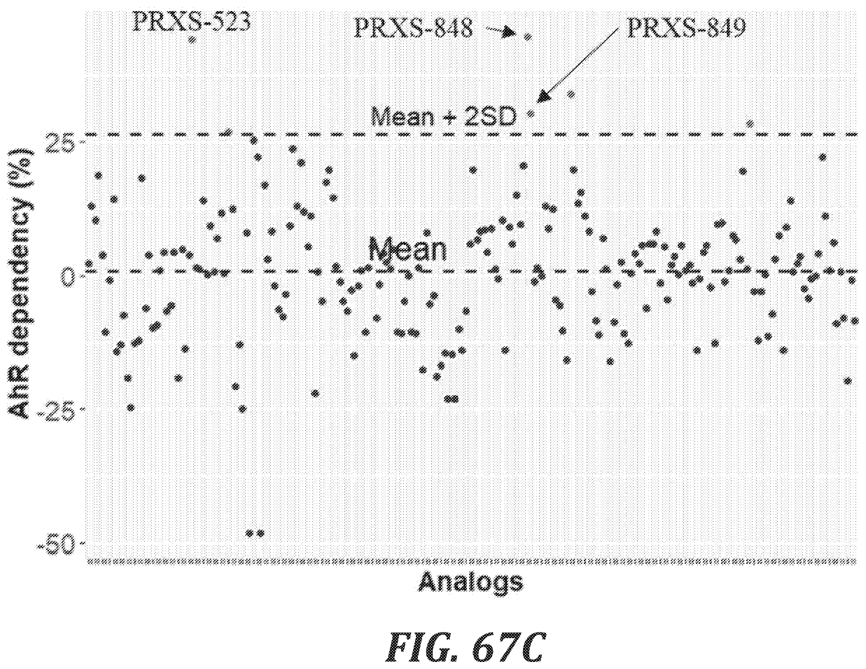

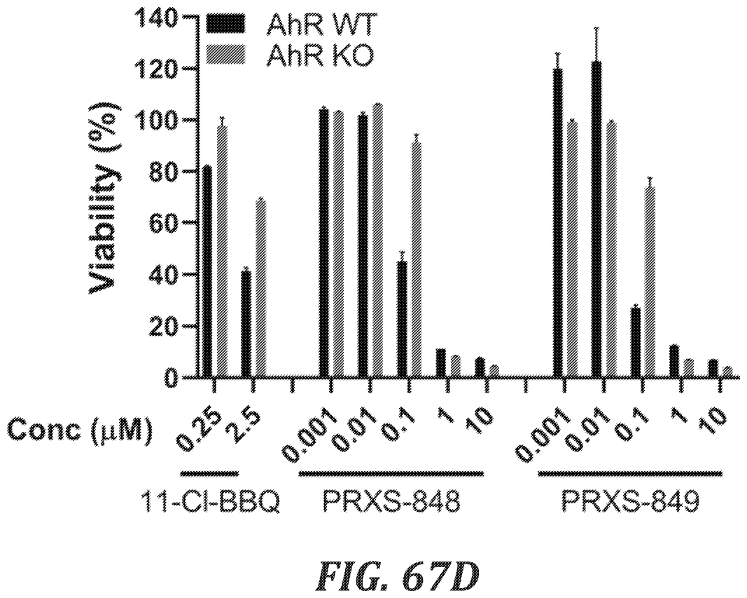

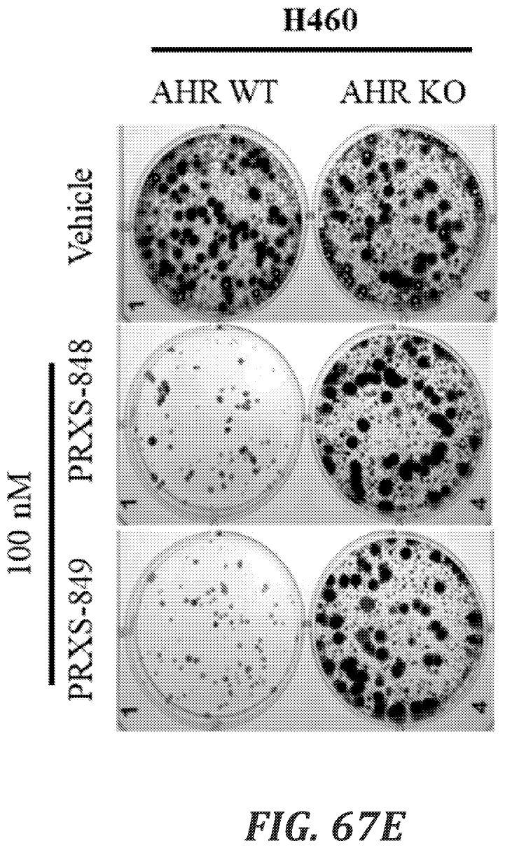

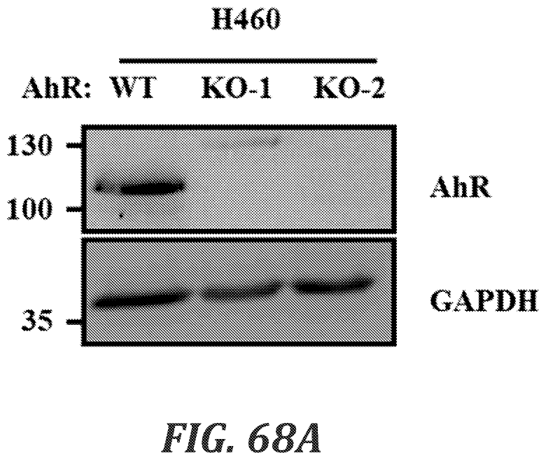

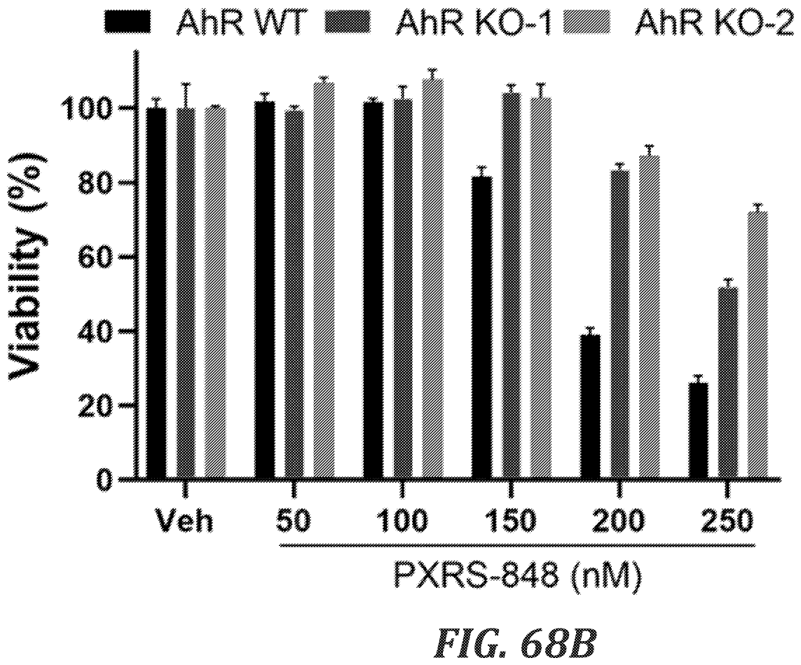

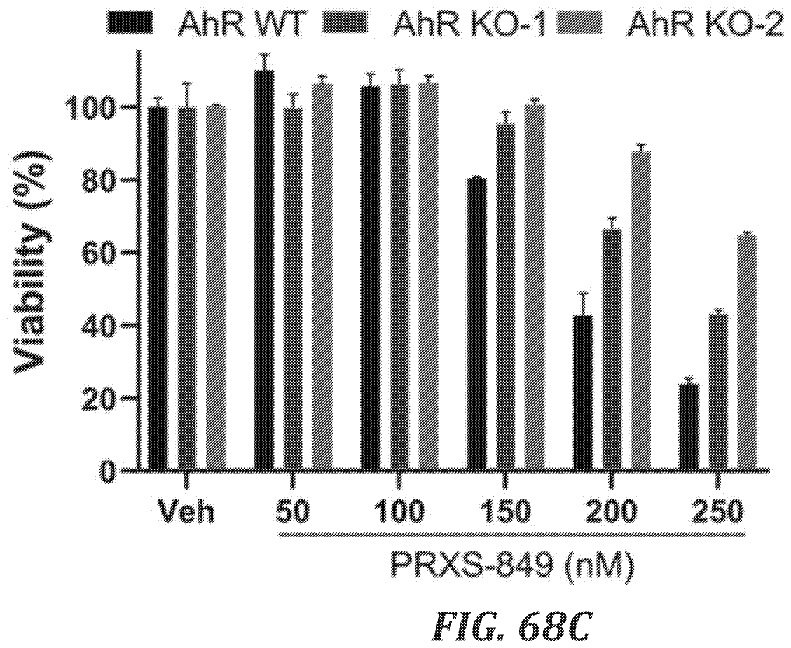

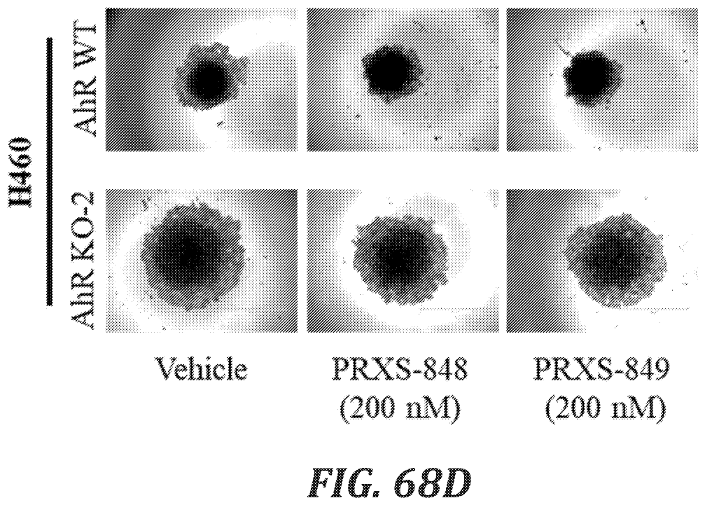

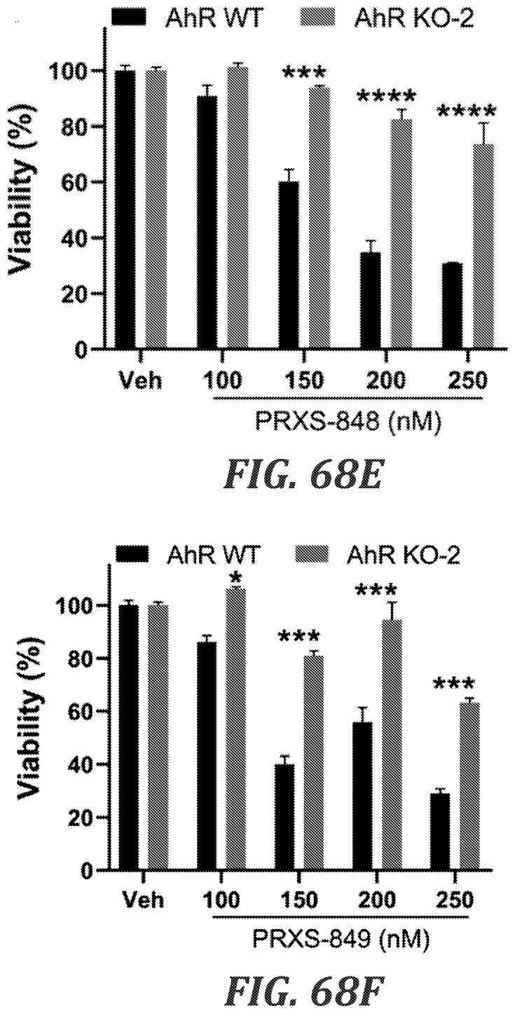

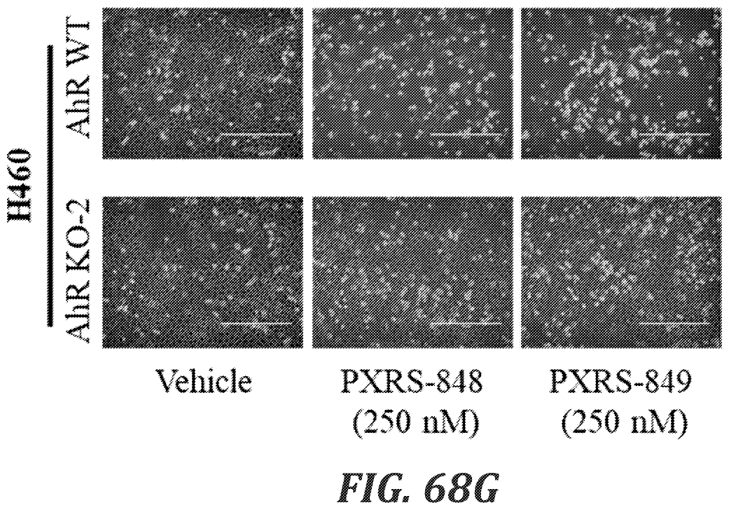

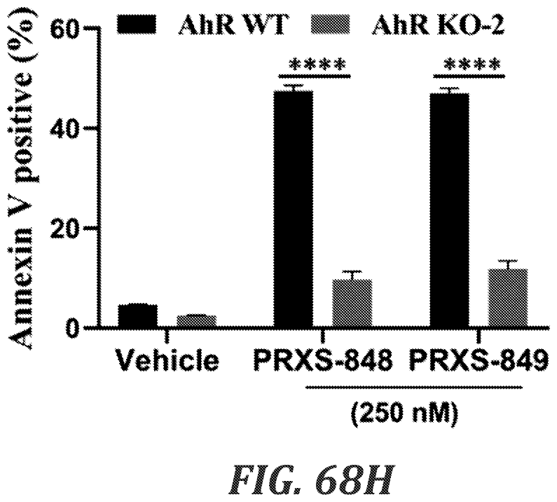

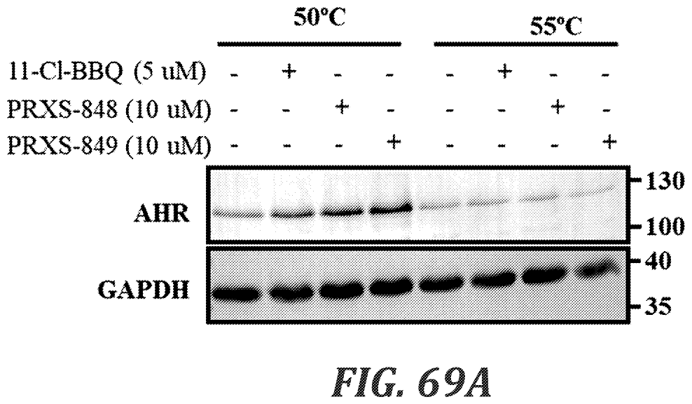

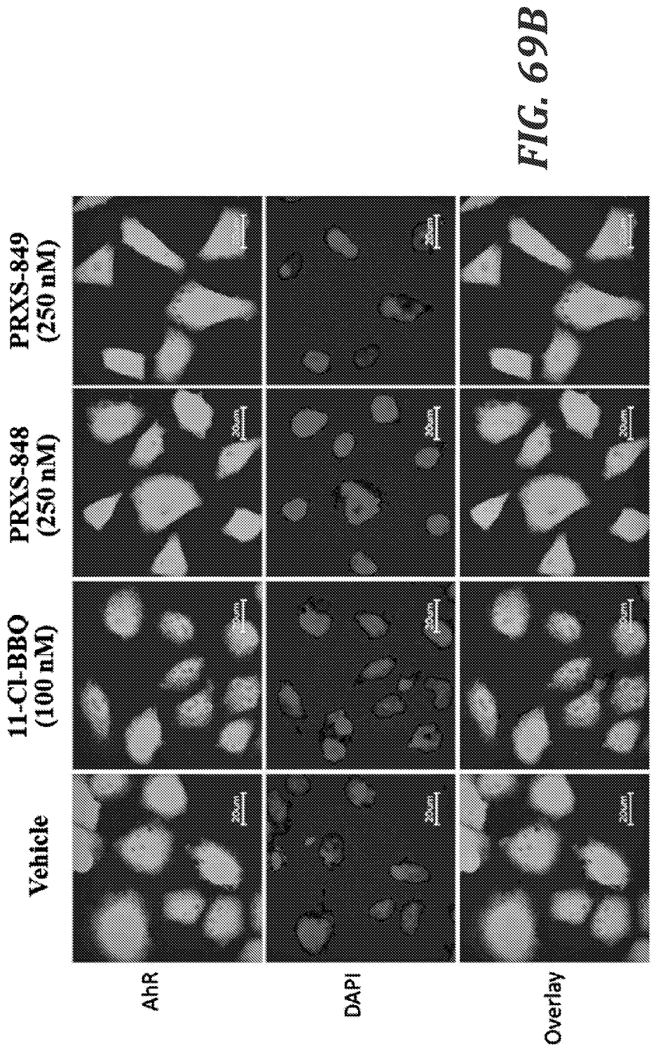

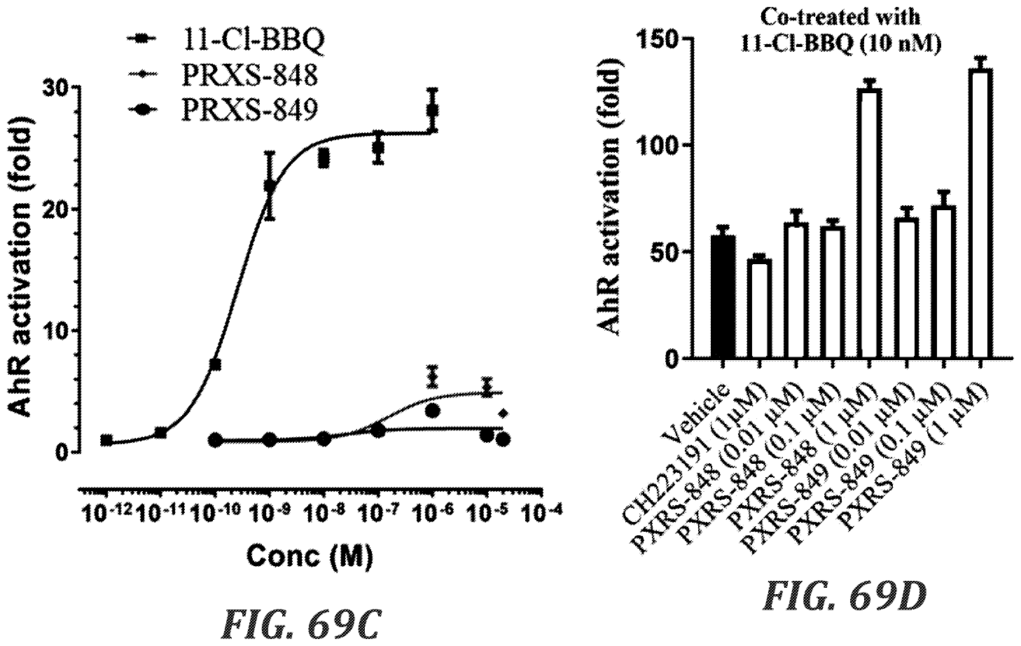

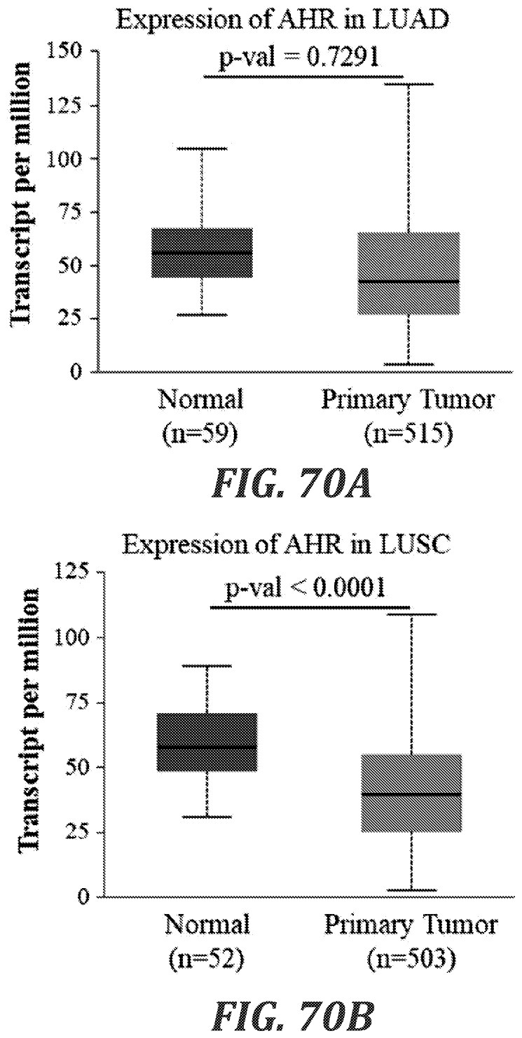

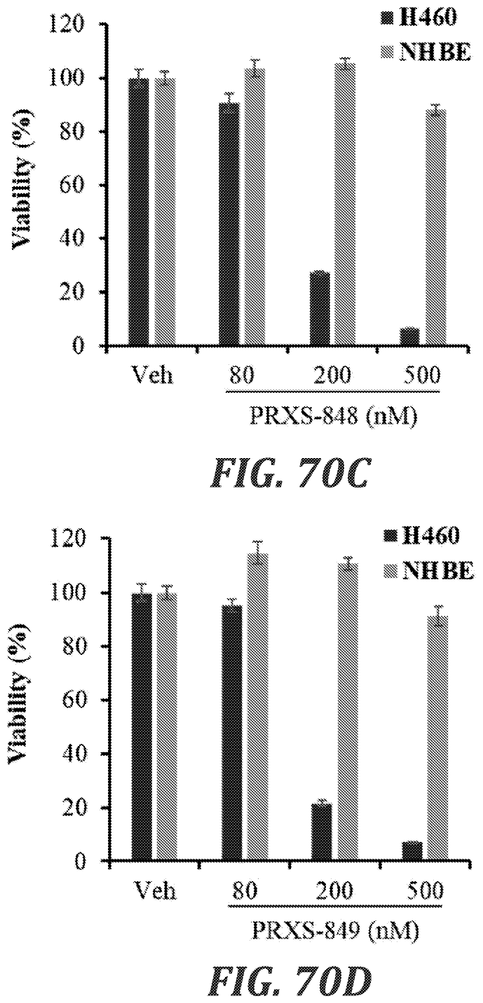

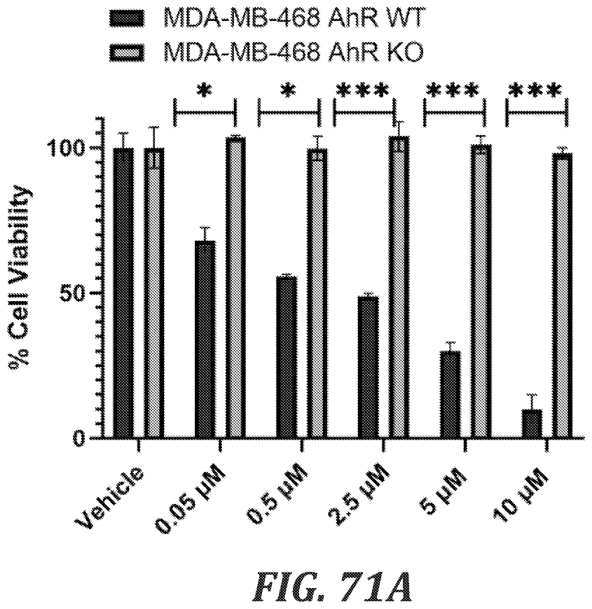

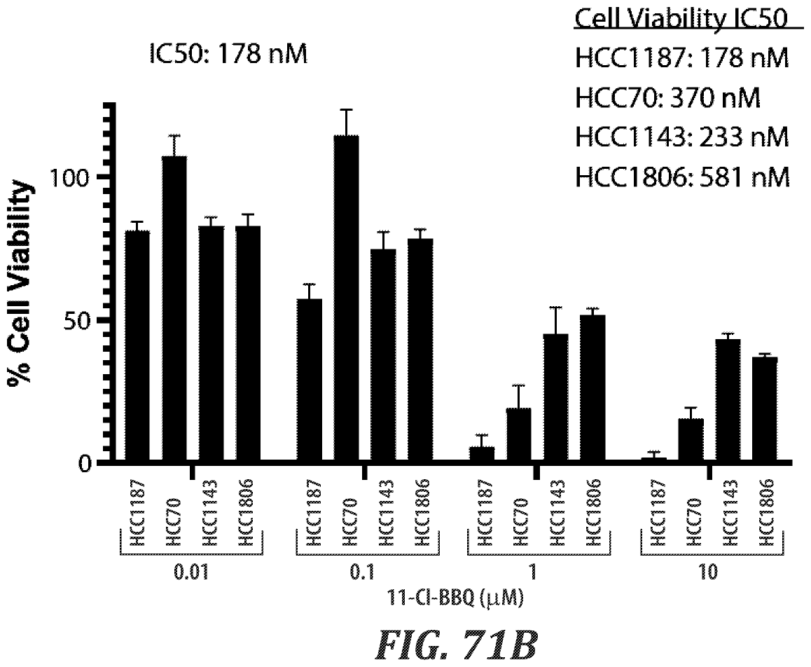

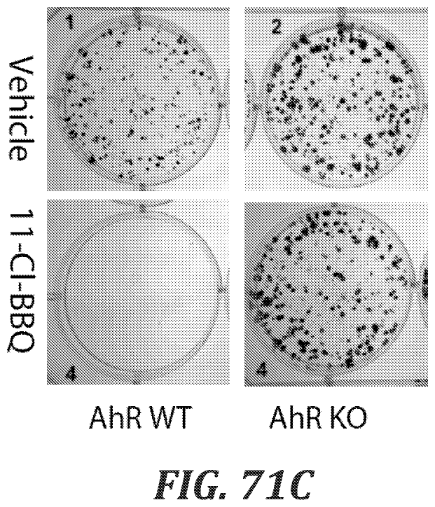

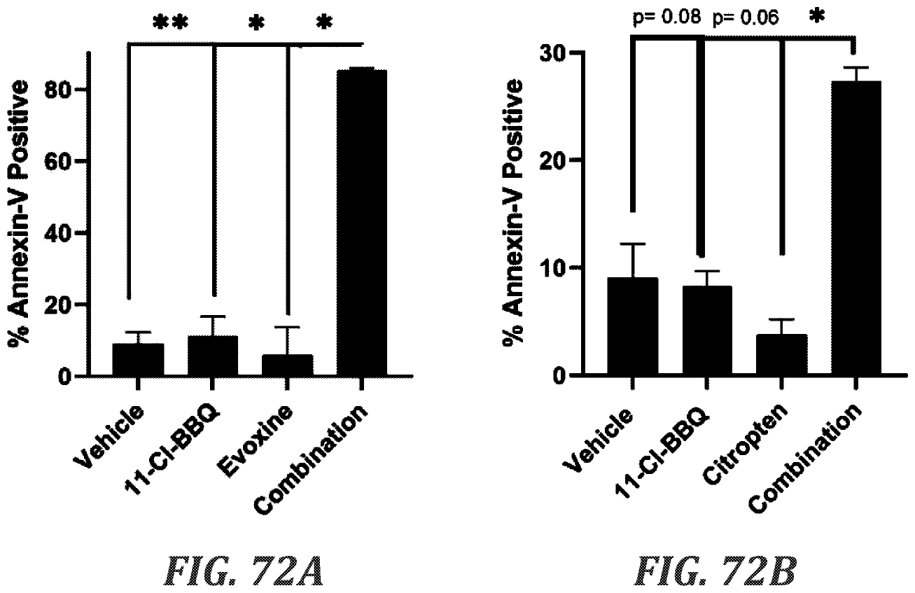

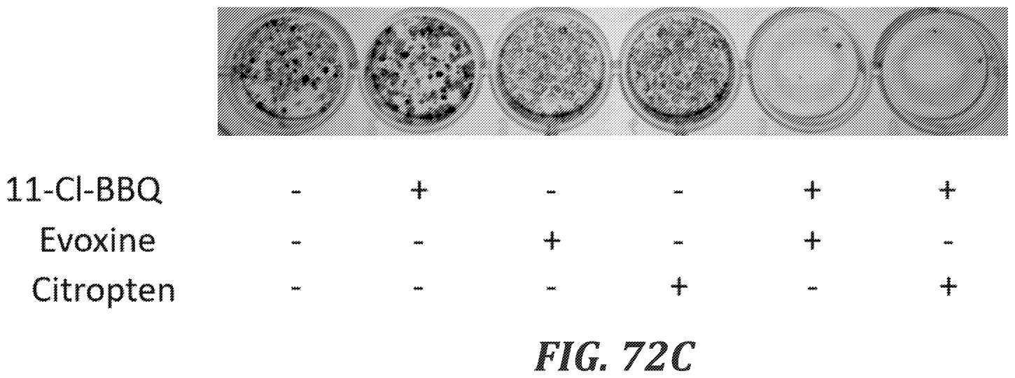

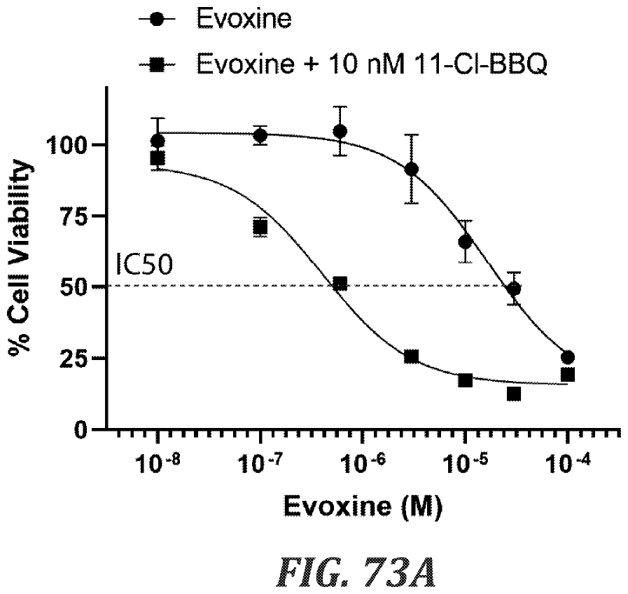

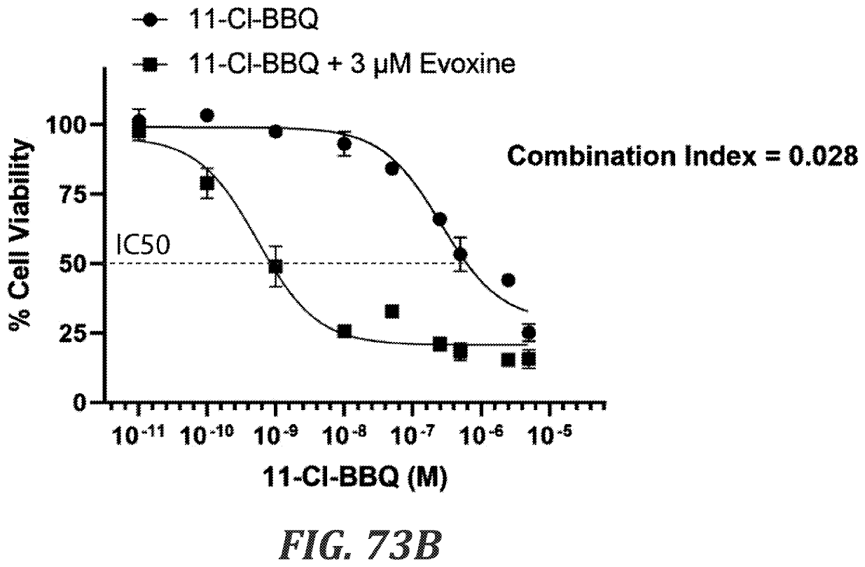

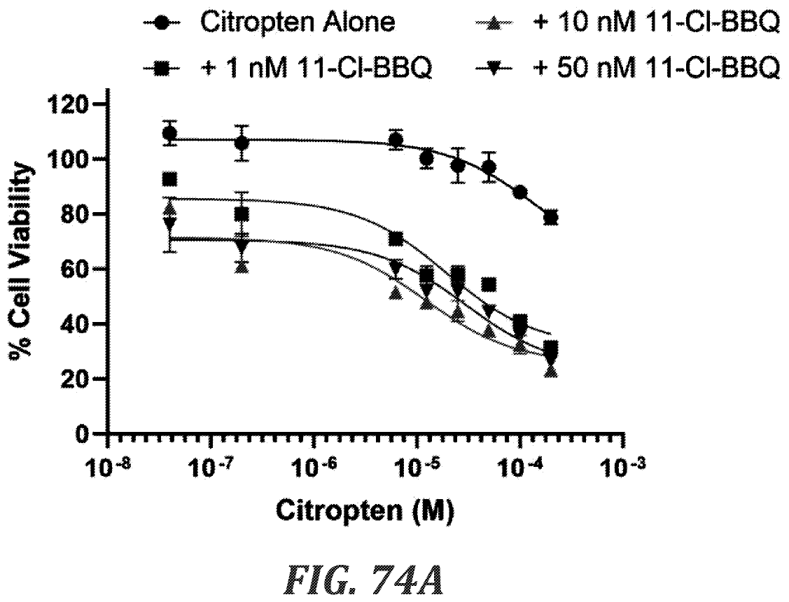

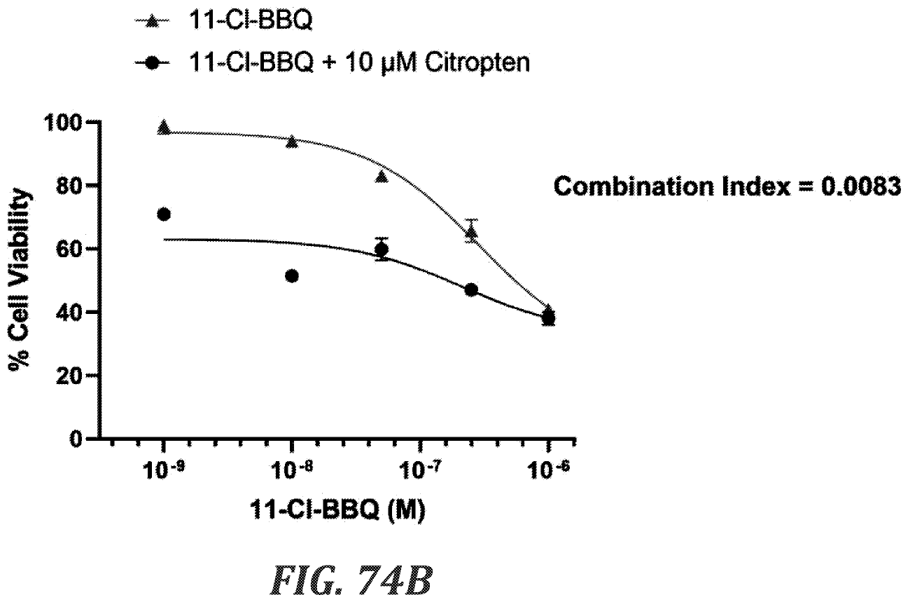

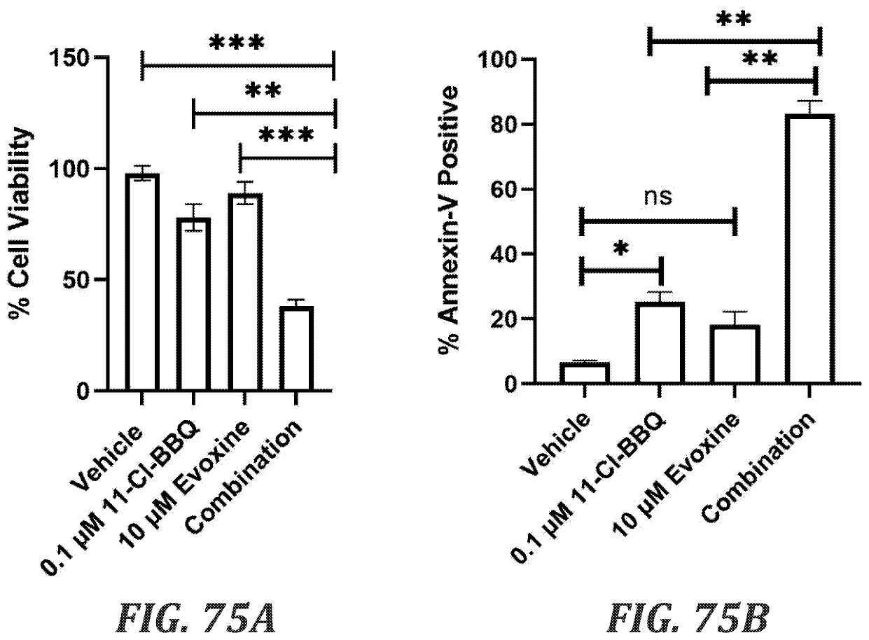

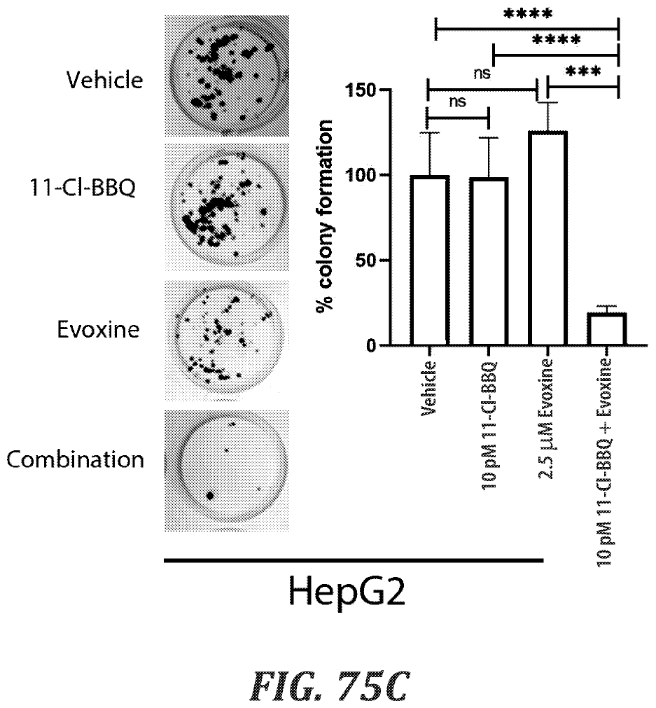

Importantly, disclosed combinations do not inhibit non-cancerous cell growth. FIG. 61 provides bar graphs of cell number (%) (for MDA-MD-468 and MCF10A cell lines) versus treatment (10 nM analog 523, 10 μM Evoxine, and 10 nM analog 523+10 μM Evoxine. The MDA-MD-468 is a cell line with epithelial morphology that was isolated from a pleural effusion of a 51-year-old Black female patient with metastatic adenocarcinoma of the breast. MCF10A is a human mammary epithelial cell line that is used for in vitro models for studying normal breast cell function and transformation. FIG. 61 shows that disclosed combinations according to the present invention, such as 10 nM analog 523+10 μM Evoxine substantially decreased the cell number for the MDA-MB-468 cell line. Conversely, the same combination had substantially no effect on the MCF10A cell line. FIG. 62 provides additional data for human mammary epithelial cells establishing that the listed treatments did not inhibit non-cancerous breast epithelial cell growth.

Other AhR compound ligands described herein include Analogs 848, 849, 860, 863, 1358, 1362, and 1389:

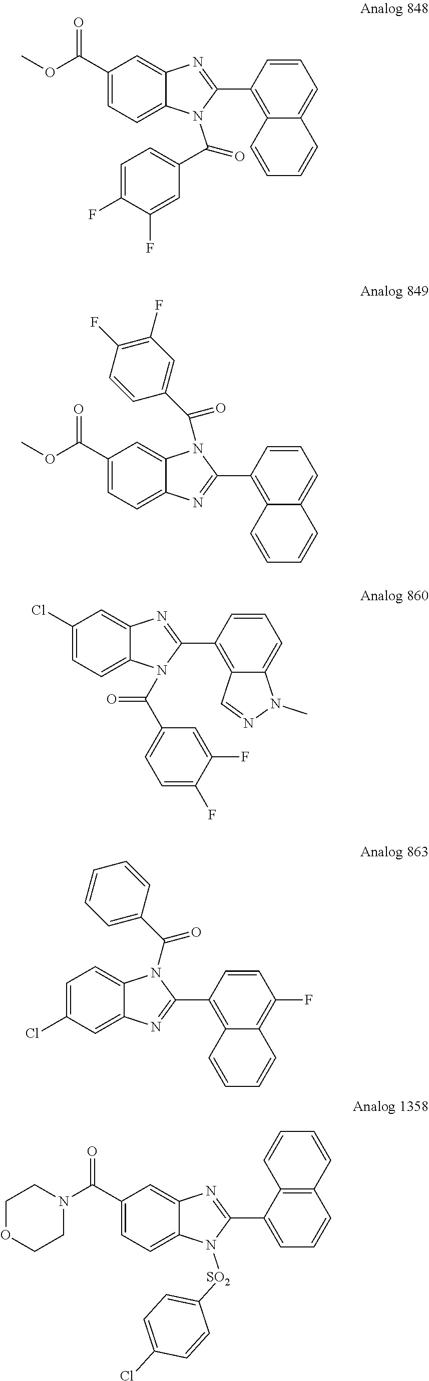 [SMILES: COC(=O)c1ccc2c(c1)nc(-c1cccc3ccccc13)n2C(=O)c1ccc(F)c(F)c1 ; COC(=O)c1ccc2nc(-c3cccc4ccccc34)n(C(=O)c3ccc(F)c(F)c3)c2c1 ; Cn1ncc2c(-c3nc4cc(Cl)ccc4n3C(=O)c3ccc(F)c(F)c3)cccc21 ; O=C(c1ccccc1)n1c(-c2ccc(F)c3ccccc23)nc2cc(Cl)ccc21 ; O=C(c1ccc2c(c1)nc(-c1cccc3ccccc13)n2S(=O)(=O)c1ccc(Cl)cc1)N1CCOCC1]

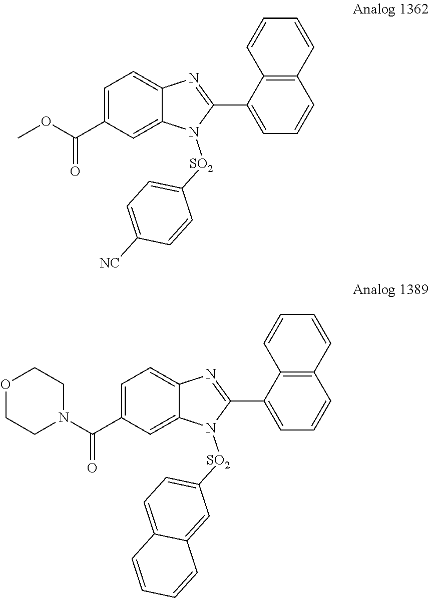 [SMILES: COC(=O)c1ccc2nc(-c3cccc4ccccc34)n(S(=O)(=O)c3ccc(C#N)cc3)c2c1 ; O=C(c1ccc2nc(-c3cccc4ccccc34)n(S(=O)(=O)c3ccc4ccccc4c3)c2c1)N1CCOCC1]

A person of ordinary skill in the art will therefore appreciate that the results presented herein are not limited to treating a single type of cancer, but instead the results are general for treating cancer generally. These cancers include, but are not limited to, lung cancer, non-small cell lung cancer, breast cancer, triple negative breast cancer, hepatocellular carcinoma (liver cancer), pancreatic cancer, urological cancer, bladder cancer, colorectal cancer, bone cancer, colon cancer, prostate cancer, renal cancer, thyroid cancer, gall bladder cancer, peritoneal cancer, ovarian cancer, cervical cancer, gastric cancer, endometrial cancer, esophageal cancer, head and neck cancer, neuroendocrine cancer, CNS cancer, brain tumors (e.g., glioma, anaplastic oligodendroglioma, adult glioblastoma multiforme, and adult anaplastic astrocytoma), bone cancer, soft tissue sarcoma, retinoblastomas, neuroblastomas, peritoneal effusions, malignant pleural effusions, mesotheliomas, Wilms tumors, trophoblastic neoplasms, hemangiopericytomas, myxoid carcinoma, round cell carcinoma, squamous cell carcinomas, esophageal squamous cell carcinomas, oral carcinomas, vulval cancer, cancers of the adrenal cortex, ACTH producing tumors, lymphoma, lung cancer, leukemia, multiple myeloma, gastrointestinal cancer, colon carcinoma, colorectal adenoma, a tumor of the neck and/or head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, Hodgkins lymphoma, Non-Hodgkins lymphoma, mammary carcinoma, follicular carcinoma, undifferentiated carcinoma, papillary carcinoma, seminoma, melanoma, IL-1 driven disorders, MyD88 driven disorders, ABC diffuse large B-cell lymphoma (DLBCL), primary cutaneous T-cell lymphoma, chronic lymphocytic leukemia, smoldering multiple myeloma, indolent multiple myeloma, hematological malignancies, leukemia, acute myeloid leukemia (AML), DLBCL, ABC DLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, myelodysplastic syndromes (MDS), myelofibrosis, polycythemia vera, Kaposi's sarcoma, Waldenström's macroglobulinemia (WM), splenic marginal zone lymphoma, multiple myeloma, plasmacytoma, intravascular large B-cell lymphoma, and drug resistant malignancies.

### IX. Methods

### 1. Cell Culture

Lung cancer cell lines A549, H460, H146, H1299 and H69AR were purchased from ATCC and maintained in RPMI 1640 medium, except the A549 cells were maintained in F12K medium, supplemented with 10% fetal bovine serum (FBS). HEK293T cells were cultured in DMEM medium supplemented with 10% FBS. Cell cultures were split twice or three times a week when cells reached 70-80% confluent. Cells were cultured at 37° C. and 5% CO₂ in a humidified cell incubator.

### 2. Viability Assays

Viability assays were performed by seeding cells at a density of 2000 cells per well in 96-well plates, and the following day, treating with compounds for 48 or 72 hours as indicated. Viability was determined by ATP-based Cell-Titer Glo Viability Assay (Promega, Madison WI). Luminescence was measured using the Tropix TR717 microplate luminometer. Viability of treated cells was determined by relativizing to vehicle (0.1-0.3% DMSO) treated wells. For profiling the activity of Analog 523 against the panel of triple-negative breast cancer cell lines, 72-hour treatments are represented.

Cancer cells were seeded in 96-well plates at a density of 3,000 cells per well in their optimal culture medium supplemented with 1% FBS overnight, then compounds were added at indicated concentrations and incubated for 72 hours; otherwise, different treatment times will be specified. Cell viability was measured using the ATP-based CellTiter Glo assay (Promega-Madison, WI) following the manufacturer's protocol. Luminescent signals were recorded using the Tropix TR717 microplate luminometer. The viability of cultures following each treatment was normalized to vehicle treatment (0.1% DMSO (v/v)).

### 3. Colony Forming Assay

H460 cells were seeded in 6-well plates at a density of 200 cell per well in complete growth media. 11-Cl-BBQ or DMSO were added the following day as indicated concentrations for two weeks in 1% FBS RMPI media, media and treatments were replenished twice a week. Cell colonies were fixed and stained with methylene blue in 50% ethanol solution overnight. Pictures of cell colonies were captured using the ChemiDoc imaging instrument, and quantified using OpenCFU software.

### 4. Primary Antibodies and Western Blot

Whole cell lysate was prepared in RIPA lysis buffer with 1× Laemmli buffer, then denatured by heating at 100° C. for 5 minutes. Denatured proteins were separated in SDS-PAGE gel and transferred onto PVDF membrane. The PVDF membrane was incubated in TBST with 5% nonfat dry milk for one hour at room temperature prior to incubating with primary antibody of interest overnight at 4° C. with gentle rotation. Appropriate horse radish peroxidase conjugated secondary antibody was applied for one hour at room temperature. Signal was developed by applying SuperSignal West Pico chemiluminescence substrate and captured using the ChemiDoc imaging instrument. Primary antibodies used in this study: anti-AHR antibody (BML-SA210-Enzo Biochem, Farmingdale, NY), anti-p27^{Kip1} antibody (610242-BD Biosciences), anti-p53 antibody (DO-1 sc-126-Santa Cruz Biotechnology, Dallas, TX), anti-p21 antibody (2947S-Cell Signaling Technology, Danvers, MA), and anti-GAPDH antibody (sc-36502, Santa Cruz).

### 5. Generation of Stable KO Cells Using CRISPR-cas9

Guide RNAs against human AhR, CDKN1B, and TP53 genes in one vector CRIPSR-cas9 lentiCRISPRv2 plasmid have been described and were purchased from GeneScript (Piscataway, NJ). Stable AhR, CDKN1B (p27) and TP53 (p53) knockout lines were generated using CRISPR-cas9 system. Briefly, for each gRNA, viral particles were generated by co-transfecting CRISPR-cas9 sgRNA plasmid with packaging plasmids psPAX2 and pMD2.G (Addgene, Watertown, MA) into HEK293T cells using lipofectamine 2000 (ThermoFisher, Waltham, MA). The viral particles were collected twice on the next two days for a total of 4 ml of supernatant containing viral particles, frozen in −80° C. overnight, filtered through 0.2 μm filter, divided into 0.5 ml aliquots and kept in −80° C. H460 or H69AR cells were transduced by reverse transduction with the viral particles in RPMI 1640 medium supplemented with 1% FBS and 10 UM protamine sulfate. Infected cells were selected for using 0.5 μg/ml puromycin for one week. Monoclonal cell lines were generated by limited dilution in 96-well plates. After 10 days in culture, cells were checked under a microscope and wells containing only one clone were selected for further expansion. Knockout phenotype was confirmed by Western Blot. Guide RNAs against AhR are known; guide RNAs targeting human p27 and p53 genes:

p27 gRNA-1
(SEQ ID NO: 1)
ATT GCT CCG CTA ACC CCG TC;
p27 gRNA-2
(SEQ ID NO: 2)
GGG TTA GCG GAG CAA TGC GC;
p27 gRNA-3
(SEQ ID NO: 3)
TTC CCC AAA TGC CGG TTC TG;
p53 gRNA-1
(SEQ ID NO: 4)
CCG GTT CAT GCC CAT GC;
p53 gRNA-2
(SEQ ID NO: 5)
CGC TAT CTG AGC GCT CA;
p53 gRNA-3
(SEQ ID NO: 6)
CCC CGG ACG ATA TTG AAC AA.

### 6. Cell Cycle Analysis

Cells were harvested by applying trypsin-EDTA then washed twice with phosphate-buffered saline (PBS) solution, fixed with 70% ethanol for 15 minutes at room temperature and kept at −20° C. overnight. Fixed samples were washed twice with PBS and stained with Hoechst 33258 (Invitrogen, Carlsbad, CA) at the final concentration of 1 μg/ml in the 0.1% Triton X-100/PBS for 20 minutes at room temperature. DNA content was analyzed by flow cytometer for 10,000 cells per sample, singlets population was selected for further analysis and cell cycle was assigned by fitting the DNA content into Dean-Jett-Fox model using FlowJo software.

### 7. Senescence-associated Beta-galactosidase (SA-βgal) Staining

H460 cells were seeded at 2×10⁵ cells/well in 6-well plate overnight and treated with indicated compounds for 5 days. Media and treatments were refreshed on the day 314. Cells were stained for senescence-associated β-galactosidase (SA-βgal) using 5-dodecanoylaminofluorescein di-β-D-galactopyranoside (C₁₂FDG). Briefly, media were aspirated and replaced with fresh media containing 0.1 μM bafilomycin A1 (Calbiochem) and incubated for 1 hour in cell culture incubator. C₁₂FDG (Setare Biotech) was added with a final concentration of 33 μM and incubated for another hour. Fluorescent signal was recorded using CytoFlex S cytometer for 10,000 cells per sample.

8. RNA Isolation and Quantitative Reverse Transcription PCR (qPCR)

Total RNA samples were isolated using the E.Z.N.A. Total RNA Kit (Omega Bio-tek, Norcross, GA) following the manufacturer's protocol. One microgram of total RNA was used to make cDNA using the Transcriptor First Strand cDNA Synthesis Kit (Roche, Basel, Switzerland) in a total volume of 20 μl. The cDNA (0.5 μl) was used for each qPCR reaction with the FastStart Universal SYBR Green Master (Rox) (Roche) in a total volume of 20 μl using standard thermal cycling on 7500 Fast real time PCR system (ABI Applied Biosystems). qPCR primers specific for human CYP1A1, CDKN1A (p21), CDKN1B (p27^{Kip1}), and GAPDH as described. qPCR primers for other genes were designed and checked for target specificity using Primer-BLAST tool; these primer sequences were: CYP1B1 forward 5′-AAG TTC TTG AGG CAC TGC GAA-3′ (SEQ ID NO: 7); reverse 5′-CCG GTA CGT TCT CCA AAT CC-3′ (SEQ ID NO:8), AHRR forward 5′-ATC GTG GAC TAT CTG GGC TTC-3′ (SEQ ID NO:9); 5′-TTC TGG TGC ATTA CAT CCG TC-3′ (SEQ ID NO:10), CABLES1 forward 5′-TCA AGA ACA TGC GGC AAC AC-3 (SEQ ID NO:11)′; reverse 5′-GGG TAC TGT CGC GAT ACG G-3′ (SEQ ID NO:12), SHISA2 forward 5′-CGG CTG CGA CAA TGA CCG-3′ (SEQ ID NO:13); reverse 5′-CAA TGA GGA ACG GCA CGT AG-3′(SEQ ID) NO: 14), ILIA forward 5′-AGT AGC AAC CAA CGG GAA GG-3′ (SBQ ID NO: 15); reverse 5′-AAG GTG CTG ACC TAG GCT TG-3′ (SEQ ID NO:16), CCNE2 forward 5′-TCA CTG ATG GTG CTT GCA GT-3′ (SEQ ID NO: 17); reverse 5′-GCC AGG AGA TGA TTG TTA CAG GA-3′ (SEQ ID NO:18), E2F7 forward 5′-TCG CTC TCC CTT CCC GAT G-3′ (SEQ ID NO:19); reverse 5′-ACC TCC ATC CCT GCT TTC CTA AG-3′ (SEQ ID NO:20)

### 9. Confirmation of AhR KO Phenotype at DNA Level

DNA from H460 CR-AHR3 clone was isolated using the G-spin Total Genomic DNA Extraction Mini Kit (iNtRON Biotechnology, South Korea) following the manufacturer's protocol. A DNA fragment flanking the binding site of the AhR sgRNA3 (5′-AAT TTC AGC GTC AGC TAC AC-3′) (SEQ ID NO:21) was amplified by a standard PCR protocol (94° C. for 2 min followed by 35 cycles of 94° C. for 30 seconds, 60° C. for 45 seconds, and 72° C. for 45 seconds, and final elongation 72° C. for 5 minutes) using the Taq polymerase (Thermofisher) with a specific primer set: forward (5′-ACT AGC AAG CAC CCA CTA ATC-3′) (SEQ ID NO: 22); reverse (5′-CCT TAG CTC ATC AGG CTC TTT-3′) (SEQ ID NO:23). PCR products were cleaned up (PCR purification kit, Qiagen) and sequenced using Sanger sequencing method at the Center for Genome research and Bioinformatic at Oregon State University.

### 10. AhR Chromatin Immunoprecipitation (ChIP)

H460 cells were seeded overnight in RPMI media supplemented with 10% FBS and were treated with 11-Cl-BBQ (2.5 M), TCDD (30 nM), or 0.1% DMSO as vehicle control for one hour. Chromatin immunoprecipitation (ChIP) was carried out using the MAGnify™ Chromatin Immunoprecipitation System (Invitrogen) following the manufacturer's protocol. Rabbit polyclonal antibody anti-AhR (BML-SA210-Enzo Biochem) and rabbit IgG provided with the ChIP kit was used as control antibody. The enrichment of AHR at CYP1A1 and p27 (CDKN1B) promoters was measured using qPCR method as described earlier. qPCR primers for p27 promoter and CYP1A1 promoter using qPCR with primers p27forward 5′-GGC CGT TTG GCT AGT TTG TT-3′ (SEQ ID NO:24); reverse 5′-GAG ATT GGC TGG TCG CGT-3′ (SEQ ID NO:25), CYP1A1 forward 5′-TGC CCA GGC GTT GCG TGA GAA G-3′ (SEQ ID NO:26); reverse 5′-ACC CGC CAC CCT TCG ACA GTT C-3′ (SEQ ID NO:27). P27 primers were designed to cover a putative AHR binding site in the promoter region of p27. CYP1A1 primers are known that are specific for the AHR binding site at CYP1A1 enhancer region.

### 11. Global Transcriptomic Study Using RNA-Seq

Total RNA samples were isolated at the indicated time using the E.Z.N.A. total RNA kit as mentioned above. The samples were quantified using a nanodrop method, and the three biological samples were combined with the same molarity and used as a representative sample for each treatment. The mixed samples were quantified using the Bioanalyzer 2100 instrument with the RNA Nano chip. All the samples had RIN numbers of 10, except one sample with a RIN of 9.9. For each sample, one microgram of total RNA was used to select for messenger RNAs using the Takara PrepX PolyA mRNA isolation kit. Libraries of mRNA were prepared using the Takara PrepX RNA-seq for Illumina Library Prep kit. The final libraries were quantified using the HS-D5000 tape on the Tapestation 420 instrument then mixed together with an equal molarity and sequenced using one lane of a Hi-Seq 3000 flow cell for 150-bp paired-end reads. RNA libraries were made and sequenced at the Center for Genome research and Bioinformatic.

RNA-seq reads were pseudo-aligned to the reference human transcriptome (release 92 from Ensembl) and quantified using kallisto with default settings. Gene set enrichment analyses (GSEA) were performed using the GSEA java application for desktop using the normalized transcript per million (TPM) values for each treatment, in AhR wild type and AhR knock out cells separately, using the single sample GSEA projection with curated canonical pathway gene sets (version v6.2).

### 12. Zebrafish Xenograft Experiments

H460 AHR wildtype (CR-V2) and knockout (CR-AHR3) cells were treated with 11-Cl-BBQ (1 and 5 μM) or 0.1% DMSO as vehicle control for 72 hours in RPMI media supplemented with 1% FBS. Cells were trypsinized, dyed with cell viable dye CM-Di1 (Thermofisher), and micro-injected into zebrafish embryos. The growth cancer xenografts were monitored at one day and four days post injection. Zebrafish were housed at the Sinnhuber Aquatic Research Laboratory at Oregon State University (Corvallis, OR, USA), and experiments were conducted according to Institutional Animal Care and Use Committee protocols.

### 13. Annexin-V Apoptosis Assay

Annexin-V staining was performed 48 hours post-treatment by harvesting cells (floating and attached) with trypsin, followed by addition of serum-containing media, and two washes with ice-cold PBS. Cells were stained with Annexin-V-PerCP conjugates (eBioscience, Waltham, MA, USA) for 15 minutes on ice before washing out residual stain and resuspending in 300 μL of binding buffer. 10,000 events were collected per sample. Data was acquired with a CytoFLEX S flow cytometer (Beckman Coulter, Brea, CA), and analyzed using the CytExpert software (Beckman Coulter).

### 14. Generation of CRISPR-Cas9 Knockout Cell Lines

Briefly, HepG2 cells were seeded in a 6-well plate and transfected for 3 d with 2.5 μg plasmids DNA using Lipofectamine 2000. Cells were grown for a week in 2 μg/ml puromycin containing media (12/8-12/15). After 11 days, cell colonies were isolated using a cloning cylinder and the AhR expression level was examined by western blot. The AhR-2 clone was used for experiments represented. pLentiCRISPR v2 control vector was a gift from Feng Zhang (Addgene plasmid #52961). The three guide RNA sequences used in this study were as follows: AhR-1, 5′-AAGTCGGTCTCTATGCCGCT-3′ (SEQ ID NO:28); AhR-2, 5′-TTGCTGCTCTACAGTTATCC-3′ (SEQ ID NO:29); AbR-3, 5′-AATTTCAGCGTCAGCTACAC-3′ (SEQ ID NO:21). MDA-MB-468 paired vector control and AhR-deficient cells were generated via the same approach described for HepG2, with the knockout clone represented derived from cells transfected with the AhR-3 gRNA.

### 15. Compound Screening

Analogs of 11-Cl-BBQ were synthesized by Praxis Precision Medicines. Screening was performed using MDA-MB-468 AhR WT and AhR KO cells previously described. Cells were seeded in 10% serum or 2% serum containing media with 1% Pen/Strep at a density of 2000 cell/well in 96-well plates. 20 hours post-seeding, cells were treated with 0.1 μM, 1 μM, or 10 μM of each analog (final DMSO concentration 0.1%-0.2%). After 48 and 72 hours, cells were harvested. The ATP-Based CellTiter Glo Assay kit (Promega, Madison, WI) was used for measuring cell viability.

### 16. Colony Assays

Cells were seeded at a density of 500 cell/well in triplicate in 6-well plates, and the following day, cells were treated with compounds and allowed to incubate for 2-3 weeks before fixation with 50% ethanol/water solution containing 0.1% methylene blue overnight. Colonies were counted manually, or with OpenCFU colony counting software, Images were acquired on a ChemiDoc imaging instrument.

### 17. Cell Cycle Analysis

Cells were seeded in media containing 10% FBS and 1% P/S at a density of 250,000 cell/well in 6 well plates. The following day, cells were treated with 100 nM 523 for 20 hours before harvesting cells by trypsinization, washing with PBS. fixation with 70% ice-cold ethanol, wash out of ethanol, followed by permeabilization with 0.1% Triton in PBS, and staining with Hoechst 33258 dye (1 μg/mL) (Invitrogen, Carlsbad, CA) for 20 minutes at room temperature. Following staining, dye was washed out with PBS before resuspension in PBS and acquisition on the flow cytometer. 10,000 events were captured per sample, and analysis of singlet population used for cell cycle distribution analysis. Manual gating was to delineate the DNA-content distribution.

### 18. Soft Agar Assay

3% agar solution was prewarmed until fully melted. 1 ml of warm media was mixed with 0.5 mL of melted 3% agar solution and added into a 6-well plate. The 1% bottom agar layer was incubated at room temperature for 30 min to solidify. Cells were detached using trypsin and resuspended in media. The 0.3% top agar was prepared by mixing 0.45 ml 1% agar medium with 1.05 ml cell suspension. The top layer was added on top of the bottom layer after it solidified. Cells were seeded at the density of 8×10³ cells/well. After placing the top agar, plates were left at room temperature for 30 min for agar to solidify, and then moved into incubator. The treatments were added 24 hours post-seeding. The cells were incubated for 30 days. Medium with treatment was added every week, once a week. After 30 days of culture, colonies were stained with 0.02% methylene blue dissolved in 50% MeOH. After one day incubation at 4 degrees Celsius, the stained colonies were imaged. Images were obtained at 20× and 40× magnification. The number of colonies in a 20× field were counted and used for quantification. Representative individual colonies were imaged at 40× for comparing size and effect.

### 19. Cell Lines

All cell lines, except for the ST1 and ST2 cells were purchased from ATCC, and maintained in 5% CO₂, at 37° C., in DMEM or RPMI media containing 10% FBS, and 1% penicillin/streptomycin. All HCC-cell lines were maintained in RPMI. All MDA-cell lines were maintained in DMEM. ZR-75I cells were maintained in RPMI. SKBR3 cells were maintained in DMEM. MCF10A cells were maintained in DMEM/F12 medium with the following supplements added: 5% horse serum, 20 ng/mL epidermal growth factor, 0.5 mg/mL hydrocortisone, 100 ng/mL cholera toxin, 10 μg/mL insulin, 1% Pen/Strep. Primary human mammary epithelial cells (HMECs) were obtained from Lonza Biosciences (Basel, Switzerland) and were maintained according to the specified culture protocols in Clonetics™ Mammary Epithelial Basal Medium (Lonza). Mouse Hepa-1 cells were kindly gifted by Dr. Michael Denison at the University of California, Davis, and were maintained in DMEM.

ST1 and ST2 cells were cultured in Mammary epithelial growth medium (Biowhitakker) (Lonza), with the following supplements: Epidermal growth factor (20 ng/mL) (Sigma), basic fibroblast growth factor (20 ng/mL) (BD Biosciences), Heparin (4 μg/mL) (Sigma), B27 Supplement (Invitrogen), Penicillin/Streptomycin (Omega), Gentamycin (35 μg/mL) (Sigma). Cells were gently passaged with Accutase solution. The isolation and characterization of the ST1 and ST2 lines have been previously described (Strietz et al., 2021).

### 20. Western Blotting

Lysates were prepared in ice-cold RIPA buffer containing Protease inhibitors (Pierce Protease Inhibitor, CAT #A32963) before addition of Laemmli buffer and boiling. Lysates were resolved on 10% SDS PAGE gels, transferred to PVDF membranes, blocked for 1 hour, and incubated with primary antibodies at the indicated concentrations in blocking solution (5% milk in Tris-buffered saline/0.1% tween) overnight at 4 degrees Celsius. Species-specific secondary antibodies conjugated to horseradish peroxidase were used to detect bound primary antibodies, and were incubated for 1 hour at room-temperature (1:2000 in block). The following antibodies were used: anti-AhR (Enzo Life Sciences, 1:1000), anti-GAPDH (sc-36502, 1:1000, Santa Cruz Biotechnologies) as loading control.

### 21. Spheroid Viability Assay

Cells were trypsinized and resuspended with 10 μL Nanoshuttle per 100,000 cells. Cells were spun at 100×g for 5 minutes then resuspended and spun again at the same speed. This was repeated twice. Cells were then seeded in a clear 96-well plate and placed on a magnetic printer for 24 hours before treatment. Treatments were added at 11× concentration to 100 μL volumes in the plate and allowed to incubate for 72 hours before adding CellTiter Glo reagent to cells and performing viability assay according to the manufacturer's instructions.

### 22. RT-qPCR

Total RNA samples were isolated using the E.Z.N.A. Total RNA Kit (Omega Bio-Tek, Norcross, GA) following the manufacturer's protocol. 1 μg of total RNA was used to make cDNA using the Transcriptor First Strand cDNA Synthesis Kit (Roche, Basel, Switzerland) in a total volume of 20 μl. The cDNA (0.5 μl) was used for each qPCR reaction with the FastStart Universal SYBR Green Master (Rox) (Roche) using standard thermal cycling conditions on a 7500 Fast real time PCR system (ABI Applied Biosystems). Primers for CYP1A1, CYP1B1 and NQO1 were previously reported.

### 23. Transcriptional Profiling by RNA-Seq

MDA-MB-468 AhR WT and AhR KO cells were seeded in 10 cm plates (3.5 million cells/plate) and allowed to attach overnight. The following day, media was replaced with fresh media containing 250 nM of Analog 523 or Vehicle (0.1% DMSO). Treatments were performed in triplicate. At 4 hours and 12 hours post-treatment, samples were harvested by addition of 700 μL of TRK-Lysis buffer and RNA isolation then performed according to the manufacturer's instructions (E.Z.N.A. Total RNA Kit, Omega Bio-Tek, Norcross, GA). Samples were quantified and 260/280 and 260/230 values inspected by NanoDrop. Samples were then sent for quality control, library preparation (poly-A enrichment) and sequencing at Novogene. Sequencing was performed with 150 bp paired-end reads to a depth of 20 million reads per cell.

### 24. Immunocytochemistry

ST2 cells were seeded in collagen-coated chamber slides (Thermo Fisher Scientific, Waltham, MA, USA), and the following day treated with Vehicle or 1 nM TCDD for 4 hours before fixation (3.7% Paraformaldehyde in PBS), permeabilization (0.1% Triton in PBS), and staining with anti-AhR antibody (Enzo Life Sciences, Farmingdale, New York) (1:400 in 1% Bovine serum albumin/PBS). Following washing, FITC-conjugated goat-anti-rabbit antibody (1:600) was incubated for 1 hour before washing and mounting with Prolong Gold Anti-fade with DAPI (Invitrogen). Images were acquired on a fluorescence microscope.

25. siRNA Knockdown of AhR

Knockdown of AhR expression with siRNA was performed as previously described (O'Donnell et. al., 2021). Confirmation of reduction in AhR protein expression was performed by Western Blotting.

### 26. Statistical Analyses

All data analyses were performed using GRAPHPAD PRISM software (San Diego, CA, USA). Bar graphs represent means±SEM, as indicated. In all experiments, statistical significance was evaluated using Student's t-test followed by Holm-Sidak test with a 95% confidence interval (α=0.05). ns=not significant P>0.05; *P≤0.05; **P≤0.01; ***P≤0.001. For siRNA knockdown experiments, two-way ANOVA was used for analysis followed by Tukey's post hoc test. IC50 values were derived using curve-fitting in GRAPHPAD PRISM software.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method for treating cancer in a subject, comprising administering to a subject in need thereof a therapeutically effective amount of an AhR ligand compound, or a stereoisomer or pharmaceutically acceptable salt thereof, and a synergizing compound, or a stereoisomer or pharmaceutically acceptable salt thereof, that, in combination with the AhR ligand compound, or a stereoisomer or pharmaceutically acceptable salt thereof, provides a synergistic anti-cancer effect to the subject.

2. A method for treating cancer in a subject, comprising administering to a therapeutically effective amount of an AhR ligand compound, or a stereoisomer or pharmaceutically acceptable salt thereof, to a subject in need thereof.

3. A method for treating cancer in a subject, comprising administering to a subject in need thereof a therapeutically effective amount of an AhR ligand compound, or a stereoisomer or pharmaceutically acceptable salt thereof, and a chemotherapeutic agent that, in combination with the AhR ligand compound, or the stereoisomer or pharmaceutically acceptable salt thereof, provides a synergistic anti-cancer effect to the subject.

4. The method of claim 1, wherein the AhR ligand compound has a Formula II 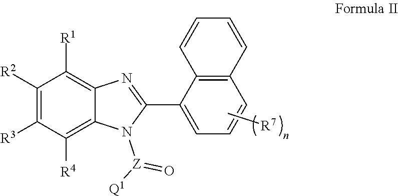
where n is 1, 2, 3, 4, 5 or 6; R¹, R², R³, and R⁴ are independently H, halogen, CN, optionally substituted C1-C10 alkyl, optionally substituted C3-C10 cycloalkyl, optionally substituted C1-C6 alkoxy, SO₂R⁵, CO₂R⁵, or CONR⁵R⁶, or any one of R¹ and R², R² and R³, and R³ and R⁴ pairs, together with the carbon atoms to which they are attached, forms an optionally substituted a five- or six-membered cycloalkenyl, heterocyclenyl, aryl or heteroaryl; R⁵ and R⁶ are independently H, optionally substituted C1-C10 alkyl, or optionally substituted C3-C10 cycloalkyl, or R⁵ and R⁶, together with the nitrogen atom to which they are attached, form an optionally substituted 5-membered ring or an optionally substituted 6-membered ring; R⁷, at each occurrence, is independently H, halogen, CN, optionally substituted C1-C10 alkyl, optionally substituted C2-C6 alkenyl, optionally substituted C1-C10 heteroalkyl, optionally substituted C1-CIO heterocyclyl, optionally substituted C6-C10 aryl, optionally substituted C5-C10 heteroaryl, optionally substituted C1-C6 alkoxy, optionally substituted C1-C6 cycloalkyloxy, OCF₃, NR⁵R⁶, SCF₃, or C(O)NR⁵R⁶; and Q1 is an optionally substituted C6-C10 aryl; optionally substituted C5-C10 heteroaryl; optionally substituted C5-C10 heterocyclyl; optionally substituted C1-C10 alkyl, or optionally substituted C3-C10 cycloalkyl.

5. The method of claim 1, wherein the AhR ligand compound has a Formula III 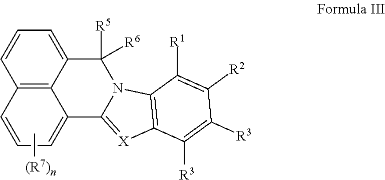
where n is 1, 2, 3, 4, 5, or 6; R¹, R², R³, and R⁴ are independently H, halogen, CN, C1-C10 alkyl, optionally substituted C3-C10 cycloalkyl, optionally substituted C1-C6 alkoxy, SO₂R⁸, CO₂R⁸, or CONR⁸R⁹, or any one of R¹ and R², R² and R³, and R³ and R⁴ pairs, together with the carbon atoms to which they are attached, forms an optionally substituted a five- or six-membered cycloalkenyl, heterocyclenyl, aryl or heteroaryl ring; R⁵ and R⁶ are independently H, halogen, OH, or C1-C6 alkyl, or R⁵ and R⁶ taken together are ═O or ═S; R⁷, at each occurrence, is independently CN, optionally substituted C1-C6 alkyl, or halogen; R⁸ and R⁹ are H, optionally substituted C1-C10 alkyl, or optionally substituted C3-C10 cycloalkyl, or R⁸ and R⁹, together with the nitrogen atom to which they are attached, form an optionally substituted 5-membered ring or an optionally substituted 6-membered ring; and n is 0, 1, 2, 3, 4, 5, or 6; and X is N or CR¹⁰, where R¹⁰ is s H, halogen, optionally substituted C6-C10 aryl; optionally substituted C5-C10 heteroaryl, or optionally substituted C1-C6 alkyl.

6. The method of claim 1, wherein the AhR ligand compound has a Formula IV 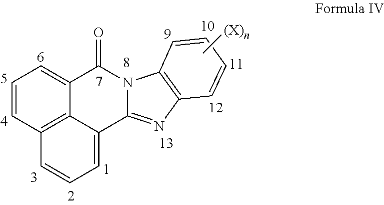
where n is 0, 1, 2, 3, or 4, and if X is present X can be located on any carbon atom or atoms of the phenyl ring and is selected from fluorine, bromine, chlorine or iodine.

7. The method of claim 1, wherein the AhR ligand compound is a benzo[de]benzoimidazoisoquinolinone, a naphthalenylbenzoimidazole, or a combination thereof.

8. The method of claim 1, wherein the AhR ligand compound is 7H-benzo[de]benzo[4,5]imidazo[2,1-a]isoquinolin-7-one, 10-halo-7H-benzo[de]benzo[4,5]imidazo[2,1-a]isoquinolin-7-one, or 11-halo-7H-benzo[de]benzo[4,5]imidazo[2,1-a]isoquinolin-7-one.

9. The method of claim 1, wherein the AhR ligand compound is

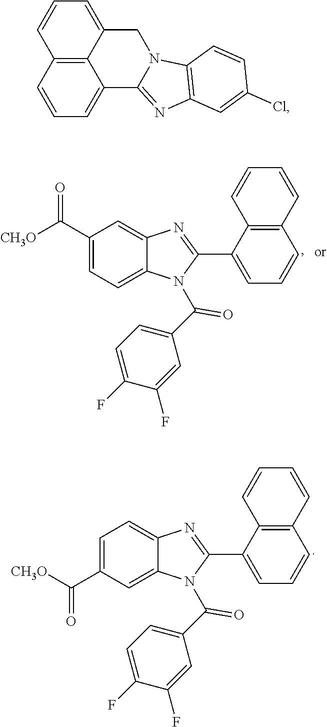

10. The method of claim 1, wherein the synergizing compound has a formula V 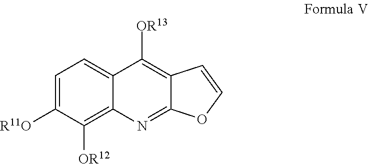
where R¹¹, R¹² and R¹³ are substituted C1-C10 alkyl, where the substituents are selected from C1-C6 alkyl, hydroxyl, and combinations thereof.

11. The method of claim 10, wherein R¹¹ substituent is a diol.

12. The method of claim 11, wherein the diol is

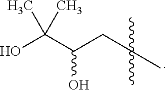 [SMILES: *CC(O)C(C)(C)O]

13. The method of claim 1, wherein the synergizing compound is evoxine having the structure

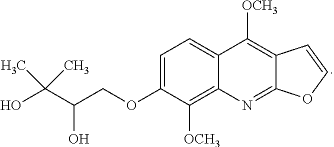 [SMILES: COc1c2ccoc2nc2c(OC)c(OCC(O)C(C)(C)O)ccc12]

14. The method of claim 1, wherein the synergizing compound is evoxine having the structure and stereochemistry below

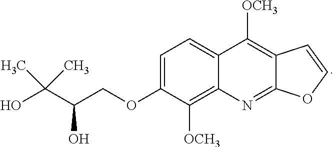 [SMILES: COc1c2ccoc2nc2c(OC)c(OC[C@@H](O)C(C)(C)O)ccc12]

15. The method of claim 1, where the synergizing compound has a formula VI 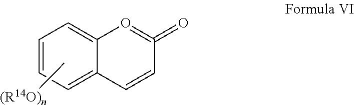
where R¹⁴ is C1-C6 alkyl, and n is 1, 2, 3 or 4.

16. The method of claim 1, wherein the synergizing compound is citropten having the structure

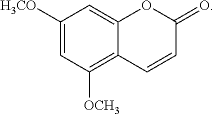 [SMILES: COc1cc(OC)c2ccc(=O)oc2c1]

17. The method of claim 1, where the synergizing compound is selected from ponatinib, melphalan, cladribine, mechloroethamine, vorinostat, ibrutinib, actinomycin D, decitabine, fludarabine, cabozantinib, dasatinib, belinostat, clofarabine, gemcitabine, pevonedistat, panobinostat, izaxomib, and teniposide.

18. (canceled)

19. The method of claim 1, wherein the cancer is lung cancer, non-small cell lung cancer, breast cancer, triple negative breast cancer, hepatocellular carcinoma (liver cancer), pancreatic cancer, urological cancer, bladder cancer, colorectal cancer, bone cancer, colon cancer, prostate cancer, renal cancer, thyroid cancer, gall bladder cancer, peritoneal cancer, ovarian cancer, cervical cancer, gastric cancer, endometrial cancer, esophageal cancer, head and neck cancer, neuroendocrine cancer, CNS cancer, brain tumors (e.g., glioma, anaplastic oligodendroglioma, adult glioblastoma multiforme, and adult anaplastic astrocytoma), bone cancer, soft tissue sarcoma, retinoblastomas, neuroblastomas, peritoneal effusions, malignant pleural effusions, mesotheliomas, Wilms tumors, trophoblastic neoplasms, hemangiopericytomas, myxoid carcinoma, round cell carcinoma, squamous cell carcinomas, esophageal squamous cell carcinomas, oral carcinomas, vulval cancer, cancers of the adrenal cortex, ACTH producing tumors, lymphoma, lung cancer, leukemia, multiple myeloma, gastrointestinal cancer, colon carcinoma, colorectal adenoma, a tumor of the neck and/or head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, Hodgkins lymphoma, Non-Hodgkins lymphoma, mammary carcinoma, follicular carcinoma, undifferentiated carcinoma, papillary carcinoma, seminoma, melanoma, IL-1 driven disorders, MyD88 driven disorders, ABC diffuse large B-cell lymphoma (DLBCL), primary cutaneous T-cell lymphoma, chronic lymphocytic leukemia, smoldering multiple myeloma, indolent multiple myeloma, hematological malignancies, leukemia, acute myeloid leukemia (AML), DLBCL, ABC DLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, myelodysplastic syndromes (MDS), myelofibrosis, polycythemia vera, Kaposi's sarcoma, Waldenström's macroglobulinemia (WM), splenic marginal zone lymphoma, multiple myeloma, plasmacytoma, intravascular large B-cell lymphoma, or a drug resistant malignancy.

20. The method of claim 1, wherein the AhR ligand compound and the synergizing compound are administered substantially simultaneously or sequentially in any order.

21. The method of claim 20, wherein the AhR ligand compound and the synergizing compound are administered as a composition.

22-42. (canceled)

43. A pharmaceutical formulation, comprising:
at least one AhR ligand compound, or a stereoisomer or pharmaceutically acceptable salt thereof;
at least one synergizing compound, or a stereoisomer or pharmaceutically acceptable salt thereof, that, in combination with the AhR ligand compound, or the stereoisomer or pharmaceutically acceptable salt thereof, provides a synergistic biological effect; and
a pharmaceutically acceptable carrier, adjuvant and/or excipient.

44-47. (canceled)
